(19) United States
(12) Patent Application Publication
(10) Pub. No.: US 2012/0282177 A1
(43) Pub. Date: Nov. 8, 2012

(54) ROR1 as Therapeutic and Diagnostic Target

(76) Inventors: Christian Rohlff, Abingdon (GB); Alasdair Stamps, Abingdon (GB); Jonathan Alexander Terrett, San Jose, CA (US)

(21) Appl. No.: 13/505,708

(22) PCT Filed: Nov. 2, 2010
(86) PCT No.: PCT/US10/55178

§ 371 Date: Jul. 11, 2012

Related U.S. Application Data
(60) Provisional application No. 61/257,320, filed on Nov. 2, 2009.
(60) Provisional application No. 61/388,694, filed on Oct. 1, 2010.

(51) Int. Cl. A61K 39/395; A61K 51/10; A61K 31/7088; A61K 31/713; C12N 5/10; C12N 15/13; C12P 21/02; C12N 1/21; C12N 1/19; C12N 1/15; G01N 33/574; C07K 16/28

(52) U.S. Cl. 424/1.49; 424/139.1; 435/7.23; 424/178.1; 514/44 A; 530/387.9; 435/69.6; 435/252.31; 435/252.33; 435/254.2; 435/254.3; 435/348; 435/252.35; 435/254.21; 435/254.23; 435/358; 435/365; 435/354; 435/353; 435/367; 435/369; 435/352; 435/357

(57) ABSTRACT

The present invention provides methods and compositions for treatment, screening, diagnosis and prognosis of cancer including bladder cancer, breast cancer, colorectal cancer, head and neck cancer, kidney cancer, liver cancer, lung cancer, ovarian cancer, pancreatic cancer, skin cancer and thyroid cancer.

### PRIORITY

The present disclosure claims the benefit of priority under 35 U.S.C. §119(e) to the following U.S. Provisional Application Ser. Nos. 61/257,320, filed Nov. 2, 2009, and 61/388,694, filed Oct. 1, 2010, both hereby incorporated by reference in their entirety.

### INTRODUCTION

The present invention relates to the identification of a membrane protein associated with cancer including bladder cancer, breast cancer, colorectal cancer, head and neck cancer, kidney cancer, liver cancer, lung cancer, ovarian cancer, pancreatic cancer, skin cancer and thyroid cancer which has utility as therapeutic target for the treatment of cancer or as a marker for such cancers. In particular the protein represents a biological target against which affinity reagents including therapeutic antibodies, or other pharmaceutical agents can be made. The invention also relates to the use of such affinity reagents for the treatment or diagnosis of cancer such as bladder cancer, breast cancer, colorectal cancer, head and neck cancer, kidney cancer, liver cancer, lung cancer, ovarian cancer, pancreatic cancer, skin cancer or thyroid cancer.

### BACKGROUND OF THE INVENTION

One of the major challenges in the treatment of cancer, including bladder cancer, breast cancer, colorectal cancer, head and neck cancer, kidney cancer, liver cancer, lung cancer, ovarian cancer, pancreatic cancer, skin cancer and thyroid cancer is to improve early detection rates, to find new non-invasive markers that can be used to follow disease progression and identify relapse, and to find improved and less toxic therapies, especially for more advanced disease, for example, where 5 year survival is still poor. There is a great need to identify targets which are more specific to the cancer cells, e.g. ones which are expressed on the surface of tumour cells so that they can be attacked by approaches such as immunotherapeutics and targeted toxins.

ROR1 is known as a receptor tyrosine kinase. Protein and mRNA analyses have demonstrated that ROR1 has two variants, (i) a full-length protein containing intracellular and extracellular domains, and (ii) a short variant lacking the extracellular domain and transmembrane regions (see Reddy et al, Oncogene, 1996 Oct. 3; 13(7): 1555-9). Literature on ROR1 has predominantly employed reagents that cannot discriminate between the two variants, for example, Baskar et al (Clin. Cancer Res., 2008 Jan. 15; 14(2): 396-404) detected both variants by Western blot, indicating that the antibody employed binds to an intracellular epitope. The presence of the full-length variant in cancer, which is required to demonstrate its utility as a cell-surface target for e.g. antibody-based cancer therapies has not previously been disclosed.

### SUMMARY OF THE INVENTION

The present invention discloses the detection of the full-length variant of tyrosine-protein kinase transmembrane receptor ROR1, hereinafter referred to as ROR1, in membrane extracts of various cancer tissues, e.g. bladder cancer, breast cancer, colorectal cancer, head and neck cancer, kidney cancer, liver cancer, lung cancer, ovarian cancer, pancreatic cancer, skin cancer or thyroid cancer but not in membrane extracts of normal tissues. This is in contrast with available mRNA data which do not discriminate between the two ROR1 variants and reveal no significant difference between the expression of ROR1 in normal and cancer tissues.

The differential expression of the full-length variant form of ROR1 in various cancers permits the extracellular domain of the protein to be targeted as the basis for affinity reagent, e.g. antibody, based therapies for such cancers. Thus the cancer-associated extracellular domain of ROR1 can be used in the generation of affinity reagents, including antibodies, that bind specifically to epitopes within such extracellular domain and can be targeted by such affinity reagents as the basis of treatment. Affinity reagents, including antibodies, that target a protein on the cell surface of cancer cells may be employed in the treatment of cancer through a variety of mechanisms, including (i) lysis by complement mediated or antibody-dependent cellular cytotoxicity (ADCC), (ii) lysis by drugs or toxin(s) conjugated to such antibodies or (iii) inhibition of the physiological function of such protein, which may be driving growth of cancer cells, e.g. through signaling pathways. An important aspect of such antibody-based treatment is that the normal expression profile of the protein target, in terms of tissue distribution and expression level, is such that any targeting of the protein target on normal tissues by the antibody does not give rise to adverse side-effects through binding to normal tissues.

The present invention demonstrates the association of the full-length variant of ROR1 with various cancers and the generation of antibodies specific to the extracellular domain of ROR1. Such antibodies have been used to verify the expression profile of ROR1 through immunohistochemistry, which reveals the presence, through specific binding, of the extracellular domain of ROR1 on the cell surface of various cancer tissues and the absence of ROR1 in normal tissues. Furthermore, such antibodies have been demonstrated to fulfill the requirements of cytolytic anti-cancer agents through their cell surface binding to cancer cell lines, internalization upon binding to cancer cell lines, live cell binding to ex-vivo malignant cells (CLL) and, crucially, their ability to kill cancer cells through internalization of a toxin.

The full-length variant of human ROR-1 is shown in SEQ ID NO: 1, this variant is characterised by and distinguished from the short length variant by the presence of the extracellular domain which is shown in SEQ ID NO: 8.

The invention provides a method for the treatment or prophylaxis of cancer wherein the extracellular domain of ROR1 is expressed in said cancer, which comprises administering to a subject in need thereof a therapeutically effective amount of an affinity reagent which binds to the extracellular domain of ROR1.

The cancer is preferably bladder cancer, breast cancer, colorectal cancer, head and neck cancer, kidney cancer, liver cancer, lung cancer, ovarian cancer, pancreatic cancer, skin cancer or thyroid cancer.

The invention also provides an affinity reagent which binds to the extracellular domain of ROR1 for use in the treatment or prophylaxis of a cancer as described above.

The invention also provides the use of an affinity reagent which binds to the extracellular domain of ROR1 in the manufacture of a medicament for the treatment or prophylaxis of a cancer as described above.

The affinity reagents for use in the invention preferably bind specifically to the extracellular domain of ROR1.

The affinity reagent may be an antibody, e.g. a whole antibody, or a functional fragment thereof or an antibody mimetic. Preferred affinity reagents included antibodies for example monoclonal antibodies.

The affinity reagent may be a chimeric antibody, a human antibody, a humanized antibody, a single chain antibody, a defucosylated antibody or a bispecific antibody.

Functional antibody fragments include is a UniBody, a domain antibody or a Nanobody.

Antibody mimetics include an Affibody, a DARPin, an Anticalin, an Avimer, a Versabody or a Duocalin.

The affinity reagents for use in the invention may contain or be conjugated to a therapeutic moiety, such as a cytotoxic moiety or a radioactive isotope. The affinity reagent may be an antibody drug conjugate or immunoconjugate.

In the methods of the invention the affinity reagent may elicit antibody-dependent cellular cytotoxicity (ADCC) or may elicit complement dependent cytotoxicity (CDC). The affinity reagent may induce apoptosis of tumor cells, kill or reduce the number of cancer stem cells and/or kill or reduce the number of circulating tumor cells. Affinity reagents may modulates the physiological function of ROR1, inhibits ligand binding and/or inhibits signal transduction pathways.

In an alternative embodiment the invention also provides a method for the treatment or prophylaxis of cancer wherein the extracellular domain of ROR1 is expressed in said cancer, which comprises administering to a subject in need thereof a therapeutically effective amount of a hybridizing agent capable of hybridizing to nucleic acid encoding extracellular domain of ROR1.

The cancer is preferably bladder cancer, breast cancer, colorectal cancer, head and neck cancer, kidney cancer, liver cancer, lung cancer, ovarian cancer, pancreatic cancer, skin cancer or thyroid cancer.

The invention also provides a hybridizing agent capable of hybridizing to nucleic acid encoding extracellular domain of ROR1 for use in the treatment or prophylaxis of a cancer as described above.

The invention also provides the use of a hybridizing agent capable of hybridizing to nucleic acid encoding extracellular domain of ROR1 in the manufacture of a medicament for the treatment or prophylaxis of a cancer as described above.

The hybridizing agents for use in the invention preferably bind specifically to nucleic acid encoding extracellular domain of ROR1.

Suitable hybridizing agent for use in the invention include inhibitory RNA, short interfering RNA (siRNA), a short hairpin RNA (shRNA), a microRNA (miRNA), and anti-sense nucleic acid or a complementary DNA (cDNA), oligonucleotodes and ribozymes.

The invention also provides a method of detecting, diagnosing and/or screening for or monitoring the progression cancer wherein the extracellular domain of ROR1 is expressed in said cancer, or of monitoring the effect of a cancer drug or therapy wherein the extracellular domain of ROR1 is expressed in said cancer, in a subject which comprises detecting the presence or level of the extracellular domain of ROR1, or one or more fragments thereof, or the presence or level of nucleic acid encoding the extracellular domain of ROR1 or which comprises detecting a change in the level thereof in said subject.

Such a method may comprise detecting the presence of the extracellular domain of ROR1, or one or more fragments thereof, or the presence of nucleic acid encoding the extracellular domain of ROR1, in which either (a) the presence of an elevated level of the extracellular domain of ROR1 or said one or more fragments thereof or an elevated level of nucleic acid encoding the extracellular domain of ROR1 in the subject as compared with the level in a healthy subject, or (b) the presence of a detectable level of extracellular domain of ROR1 or said one or more fragments thereof or a detectable level of nucleic acid encoding extracellular domain of ROR1 in the subject as compared with a corresponding undetectable level in a healthy subject is indicative of the presence of cancer wherein the extracellular domain of ROR1 is expressed in said cancer, in said subject.

The invention also provides a method of detecting, diagnosing and/or screening for or monitoring the progression cancer wherein the extracellular domain of ROR1 is expressed in said cancer, or of monitoring the effect of a cancer drug or therapy wherein the extracellular domain of ROR1 is expressed in said cancer, in a subject which comprises detecting the presence or level of antibodies capable of immunospecific binding to the extracellular domain of ROR1, or one or more fragments thereof.

In such methods the cancer is preferably bladder cancer, breast cancer, colorectal cancer, head and neck cancer, kidney cancer, liver cancer, lung cancer, ovarian cancer, pancreatic cancer, skin cancer or thyroid cancer.

In the diagnostic methods according to the invention the presence of the extracellular domain of ROR1, or one or more fragments thereof, or the presence of nucleic acid encoding the extracellular domain of ROR1, or the presence or level of antibodies capable of immunospecific binding to the extracellular domain of ROR1, or one or more fragments thereof, is detected by analysis of a biological sample obtained from the subject.

The presence of the extracellular domain of ROR1, or one or more fragments thereof, may be detected using an affinity reagent which binds to the extracellular domain of ROR1. The affinity reagent may be any suitable affinity reagent as mentioned above. The affinity reagent may contain or be conjugated to a detectable label.

In any of the aspects of the invention referred to above the subject may be a human.

The invention also provides a methods for identifying an agent for the treatment or prophylaxis of cancer wherein the extracellular domain of ROR1 is expressed in said cancer, wherein the method comprises (a) contacting the extracellular domain of ROR1, or one or more fragments thereof, with a candidate agent; and (b) determining whether the agent binds to the extracellular domain of ROR1, or one or more fragments thereof. The method may also further comprise the step of testing the ability of an agent which binds to the extracellular domain of ROR1, or one or more fragments thereof, to inhibit cancer wherein the extracellular domain of ROR1 is expressed in said cancer.

The agents identifies using the method according to this aspect of the invention are preferably for the treatment or prophylaxis of bladder cancer, breast cancer, colorectal cancer, head and neck cancer, kidney cancer, liver cancer, lung cancer, ovarian cancer, pancreatic cancer, skin cancer or thyroid cancer.

The agents identified using such methods may be small molecules and may modulate the activity of ROR1, reduce ligand binding to the extracellular domain of ROR1 or reduce ROR1 dimerisation.

In the various embodiments of the invention described herein particular cancer types which may be mentioned are bladder cancer, colorectal cancer, head and neck cancer, kidney cancer, liver cancer, ovarian cancer, pancreatic cancer, skin cancer or thyroid cancer, for example bladder cancer, colorectal cancer, head and neck cancer, liver cancer, ovarian cancer, pancreatic cancer, skin cancer or thyroid cancer.

In one embodiment the cancer to be detected, prevented or treated is bladder cancer.

In another embodiment the cancer to be detected, prevented or treated is breast cancer.

In another embodiment the cancer to be detected, prevented or treated is colorectal cancer.

In another embodiment the cancer to be detected, prevented or treated is head and neck cancer.

In another embodiment the cancer to be detected, prevented or treated is kidney cancer.

In another embodiment the cancer to be detected, prevented or treated is liver cancer.

In another embodiment the cancer to be detected, prevented or treated is lung cancer.

In another embodiment the cancer to be detected, prevented or treated is ovarian cancer.

In another embodiment the cancer to be detected, prevented or treated is pancreatic cancer.

In another embodiment the cancer to be detected, prevented or treated is skin cancer.

In another embodiment the cancer to be detected, prevented or treated is thyroid cancer.

Other aspects of the present invention are set out below and in the claims herein.

### BRIEF DESCRIPTION OF THE FIGURES

FIGS. 1a and 1b show the amino acid sequence of ROR1 identified using 1-D gel electrophoresis. The tandem peptides detected experimentally by mass spectrometry are underlined.

FIGS. 2a, 2b, and 2c show the amino acid sequence of ROR1 identified using iTRAQ. The tandem peptides detected experimentally by mass spectrometry are underlined.

FIG. 3 shows the results of IHC analysis in a high density array of the 19 most common types of cancer. Results indicate the % prevalence and the staining at different intensities (+, ++, +++) for each tumor type.

FIGS. 4a and 4b show the RNA profiling of ROR1 using cancer cell lines, cancer tissues and normal tissues.

FIGS. 5a and 5b show the flow cytometry analysis of anti-ROR1 monoclonal antibodies, indicating the specific binding of those antibodies to the human lung adenocarcinoma cell line, A549, small cell lung cancer cell line, H69, and colon carcinoma cell line, HT-29.

FIGS. 6a to 6d show the internalization of anti-ROR1 monoclonal antibodies by HEK293, H69, HT-29 and A549 cells, using MabZAP assay.

### DETAILED DESCRIPTION OF THE INVENTION

In the detailed description below unless the context requires otherwise references to ROR1 are references to the long variant as defined by the presence of the extracellular domain.

The invention described in detail below encompasses the administration of therapeutic compositions to a subject, e.g. a mammalian subject, to treat or prevent cancer e.g. bladder cancer, breast cancer, colorectal cancer, head and neck cancer, kidney cancer, liver cancer, lung cancer, ovarian cancer, pancreatic cancer, skin cancer or thyroid cancer. The invention also provides methods and compositions for clinical screening, diagnosis and prognosis of cancer e.g. bladder cancer, breast cancer, colorectal cancer, head and neck cancer, kidney cancer, liver cancer, lung cancer, ovarian cancer, pancreatic cancer, skin cancer or thyroid cancer in a mammalian subject for identifying patients most likely to respond to a particular therapeutic treatment, for monitoring the results of cancer e.g. bladder cancer, breast cancer, colorectal cancer, head and neck cancer, kidney cancer, liver cancer, lung cancer, ovarian cancer, pancreatic cancer, skin cancer or thyroid cancer therapy, for drug screening and drug development.

In one aspect the invention provides an agent capable of specific binding to ROR1, or a fragment thereof, or a hybridising agent capable of hybridizing to nucleic acid encoding ROR1 or an agent capable of detecting the activity of ROR1 for use in treating, screening for, detecting and/or diagnosing disease, such as cancer, and especially bladder cancer, breast cancer, colorectal cancer, head and neck cancer, kidney cancer, liver cancer, lung cancer, ovarian cancer, pancreatic cancer, skin cancer and/or thyroid cancer.

Another aspect of the invention is an affinity reagent capable of specific binding to ROR1 or a fragment thereof, for example an affinity reagent which contains or is conjugated to a detectable label or contains or is conjugated to a therapeutic moiety such as a cytotoxic moiety. The affinity reagent may, for example, be an antibody.

The affinity reagents for use in the invention may bind to an epitope on the extracellular domain of ROR1 which comprises one or more of the portions of SEQ ID NO: 1 defined by amino acids 30-76, 88-92, 105-111, 137-142, 149-165, 187-193, 213-218, 227-246, 254-266, 278-297, 303-319, 325-332, 338-360, 367-371 or 377-403.

Another aspect of the invention is a pharmaceutical composition comprising a therapeutically effective amount of an affinity reagent capable of specific binding to ROR1 or a fragment thereof.

In another aspect the invention provides use of a ROR1 polypeptide, or one or more fragments or derivatives thereof, for the treatment or prophylaxis of e.g. bladder cancer, breast cancer, colorectal cancer, head and neck cancer, kidney cancer, liver cancer, lung cancer, ovarian cancer, pancreatic cancer, skin cancer and/or thyroid cancer.

The invention also provides use of a ROR1 polypeptide, one or more fragments or derivatives thereof in the manufacture of a medicament for the treatment or prophylaxis of e.g. bladder cancer, breast cancer, colorectal cancer, head and neck cancer, kidney cancer, liver cancer, lung cancer, ovarian cancer, pancreatic cancer, skin cancer and/or thyroid cancer.

In one aspect there is provided a method of treatment comprising administering a therapeutically effective amount of a ROR1 polypeptide, one or more fragments or derivatives thereof, or one or more fragments or derivatives thereof, for the treatment or prophylaxis of e.g. bladder cancer, breast cancer, colorectal cancer, head and neck cancer, kidney cancer, liver cancer, lung cancer, ovarian cancer, pancreatic cancer, skin cancer and/or thyroid cancer.

The invention further provides a method for the treatment or prophylaxis of e.g. bladder cancer, breast cancer, colorectal cancer, head and neck cancer, kidney cancer, liver cancer, lung cancer, ovarian cancer, pancreatic cancer, skin cancer and/or thyroid cancer in a subject, or of vaccinating a subject against e.g. bladder cancer, breast cancer, colorectal cancer, head and neck cancer, kidney cancer, liver cancer, lung cancer, ovarian cancer, pancreatic cancer, skin cancer and/or thyroid cancer, which comprises the step of administering to the subject an effective amount of a ROR1 polypeptide and/or one or more antigenic or immunogenic fragments thereof, for example as a vaccine.

The mammalian subject may be a non-human mammal, but is generally human, such as a human adult, i.e. a human subject at least 21 (for example at least 35, at least 50, at least 60, at least 70, or at least 80) years old.

In one aspect there is provided a composition capable of eliciting an immune response in a subject, which composition comprises a ROR1 polypeptide and/or one or more antigenic or immunogenic fragments thereof, and one or more suitable adjuvants (suitable adjuvants are discussed below).

The composition capable of eliciting an immune response may for example be provided as a vaccine comprising a ROR1 polypeptide or derivatives thereof, and/or one or more antigenic or immunogenic fragments thereof.

For clarity of disclosure, and not by way of limitation, the invention will be described with respect to the analysis of bladder, breast, colorectal, head and neck, kidney, liver, lung, ovarian, pancreatic, skin or thyroid tissue. However, as one skilled in the art will appreciate, the assays and techniques described below can be applied to other types of patient samples, including body fluids (e.g. blood, urine or saliva), a tissue sample from a patient at risk of having bladder cancer, breast cancer, colorectal cancer, head and neck cancer, liver cancer, lung cancer, ovarian cancer, pancreatic cancer, skin cancer or thyroid cancer (e.g. a biopsy such as a bladder, breast, colorectal, head and neck, liver, lung, ovarian, pancreatic, skin or thyroid biopsy) or homogenate thereof. The methods and compositions of the present invention are specially suited for screening, diagnosis and prognosis of a living subject, but may also be used for postmortem diagnosis in a subject, for example, to identify family members at risk of developing the same disease.

In some embodiments, the present invention is a method for preparing an anti-ROR1 antibody, said method comprising the steps of: obtaining a host cell that contains one or more nucleic acid molecules encoding the antibody of the invention; growing the host cell in a host cell culture; providing host cell culture conditions wherein the one or more nucleic acid molecules are expressed; and recovering the antibody from the host cell or from the host cell culture.

Other aspects of the invention are directed to methods of making the antibodies of the invention, comprising the steps of: immunizing a transgenic animal comprising human immunoglobulin genes with a ROR1 peptide; recovering B-cells from said transgenic animal; making hybridomas from said B-cells; selecting hybridomas that express antibodies that bind ROR1; and recovering said antibodies that bind ROR1 from said selected hybridomas.

In other embodiments, the method of making anti-ROR1 antibodies, comprises the steps of:

immunizing a transgenic animal comprising human immunoglobulin genes with a ROR1 peptide;

recovering mRNA from the B cells of said transgenic animal;

converting said mRNA to cDNA;

expressing said cDNA in phages such that anti-ROR1 antibodies encoded by said cDNA are presented on the surface of said phages;

selecting phages that present anti-ROR1 antibodies;

recovering nucleic acid molecules from said selected phages that encode said anti-ROR1 immunoglobulins;

expressing said recovered nucleic acid molecules in a host cell; and

recovering antibodies from said host cell that bind ROR1.

Another aspect of the invention provides use of a ROR1 polypeptide, one or more immunogenic fragments or derivatives thereof for the treatment or prophylaxis of e.g. bladder cancer, breast cancer, colorectal cancer, head and neck cancer, kidney cancer, liver cancer, lung cancer, ovarian cancer, pancreatic cancer, skin cancer or thyroid cancer.

In another aspect, the invention provides methods of treating e.g. bladder cancer, breast cancer, colorectal cancer, head and neck cancer, kidney cancer, liver cancer, lung cancer, ovarian cancer, pancreatic cancer, skin cancer or thyroid cancer, comprising administering to a patient a therapeutically effective amount of a compound that modulates (e.g., upregulates or downregulates) or complements the expression or the biological activity (or both) of the protein of the invention in patients having e.g. bladder cancer, breast cancer, colorectal cancer, head and neck cancer, kidney cancer, liver cancer, lung cancer, ovarian cancer, pancreatic cancer, skin cancer or thyroid cancer, in order to (a) prevent the onset or development of e.g. bladder cancer, breast cancer, colorectal cancer, head and neck cancer, kidney cancer, liver cancer, lung cancer, ovarian cancer, pancreatic cancer, skin cancer or thyroid cancer; (b) prevent the progression of e.g. bladder cancer, breast cancer, colorectal cancer, head and neck cancer, kidney cancer, liver cancer, lung cancer, ovarian cancer, pancreatic cancer, skin cancer or thyroid cancer; or (c) ameliorate the symptoms of e.g. bladder cancer, breast cancer, colorectal cancer, head and neck cancer, kidney cancer, liver cancer, lung cancer, ovarian cancer, pancreatic cancer, skin cancer or thyroid cancer.

According to another aspect of the invention we provide a method of detecting, diagnosing and/or screening for or monitoring the progression of e.g. bladder cancer, breast cancer, colorectal cancer, head and neck cancer, kidney cancer, liver cancer, lung cancer, ovarian cancer, pancreatic cancer, skin cancer or thyroid cancer or of monitoring the effect of e.g. an anti-bladder cancer, anti-breast cancer, anti-colorectal cancer, anti-head and neck cancer, anti-kidney cancer, anti-liver cancer, anti-lung cancer, anti-ovarian cancer, anti-pancreatic cancer, anti-skin cancer or anti-thyroid cancer drug or therapy in a subject which comprises detecting the presence or level of ROR1, or one or more fragments thereof, or the presence or level of nucleic acid encoding ROR1 or the presence or level of the activity of ROR1 or which comprises detecting a change in the level thereof in said subject.

According to another aspect of the invention we provide a method of detecting, diagnosing and/or screening for e.g. bladder cancer, breast cancer, colorectal cancer, head and neck cancer, kidney cancer, liver cancer, lung cancer, ovarian cancer, pancreatic cancer, skin cancer or thyroid cancer in a candidate subject which comprises detecting the presence of ROR1, or one or more fragments thereof, or the presence of nucleic acid encoding ROR1 or the presence of the activity of ROR1 in said candidate subject, in which either (a) the presence of an elevated level of ROR1 or said one or more fragments thereof or an elevated level of nucleic acid encoding ROR1 or the presence of an elevated level of ROR1 activity in the candidate subject as compared with the level in a healthy subject or (b) the presence of a detectable level of ROR1 or said one or more fragments thereof or a detectable level of nucleic acid encoding ROR1 or the presence of a detectable level of ROR1 activity in the candidate subject as compared with a corresponding undetectable level in a healthy subject indicates the presence of e.g. bladder cancer, breast cancer, colorectal cancer, head and neck cancer, kidney cancer, liver cancer, lung cancer, ovarian cancer, pancreatic cancer, skin cancer or thyroid cancer in said subject.

According to another aspect of the invention we provide a method of monitoring the progression of e.g. bladder cancer, breast cancer, colorectal cancer, head and neck cancer, kidney cancer, liver cancer, lung cancer, ovarian cancer, pancreatic cancer, skin cancer or thyroid cancer in a subject or of monitoring the effect of e.g. an anti-bladder cancer, anti-breast cancer, anti-colorectal cancer, anti-head and neck cancer, anti-kidney cancer, anti-liver cancer, anti-lung cancer, anti-ovarian cancer, anti-pancreatic cancer, anti-skin cancer or anti-thyroid cancer drug or therapy which comprises detecting the presence of ROR1, or one or more fragments thereof, or the presence of nucleic acid encoding ROR1 or the presence of the activity of ROR1 in said candidate subject at a first time point and at a later time point, the presence of an elevated or lowered level of ROR1 or said one or more fragments thereof or an elevated or lowered level of nucleic acid encoding ROR1 or the presence of an elevated or lowered level of ROR1 activity in the subject at the later time point as compared with the level in the subject at said first time point, indicating the progression or regression of e.g. bladder cancer, breast cancer, colorectal cancer, head and neck cancer, kidney cancer, liver cancer, lung cancer, ovarian cancer, pancreatic cancer, skin cancer or thyroid cancer or indicating the effect or non-effect of e.g. an anti-bladder cancer, anti-breast cancer, anti-colorectal cancer, anti-head and neck cancer, anti-kidney cancer, anti-liver cancer, anti-lung cancer, anti-ovarian cancer, anti-pancreatic cancer, anti-skin cancer or anti-thyroid cancer drug or therapy in said subject.

ROR1 full-length protein is expressed in fetal tissues at various loci and developmental stages, with little or no expression persisting in other adult tissues. It is selectively overexpressed in CLL, at up to 20,000 copies/cell (Clinical Cancer Research 2008 14:396-404), where the target has been shown to internalize. At these levels, an antibody with enhanced ADCC engineering, or an antibody-drug conjugate (ADC), would be therapeutically effective.

According to another aspect of the invention there is provided a method of detecting, diagnosing and/or screening for or monitoring the progression of cancer e.g. bladder cancer, breast cancer, colorectal cancer, head and neck cancer, kidney cancer, liver cancer, lung cancer, ovarian cancer, pancreatic cancer, skin cancer or thyroid cancer or of monitoring the effect of an anti-cancer e.g. anti-bladder cancer, anti-breast cancer, anti-colorectal cancer, anti-head and neck cancer, anti-kidney cancer, anti-liver cancer, anti-lung cancer, anti-ovarian cancer, anti-pancreatic cancer, anti-skin cancer or anti-thyroid cancer drug or therapy in a subject which comprises detecting the presence or level of antibodies capable of immunospecific binding to ROR1, or one or more epitope-containing fragments thereof or which comprises detecting a change in the level thereof in said subject.

According to another aspect of the invention there is also provided a method of detecting, diagnosing and/or screening for cancer e.g. bladder cancer, breast cancer, colorectal cancer, head and neck cancer, kidney cancer, liver cancer, lung cancer, ovarian cancer, pancreatic cancer, skin cancer or thyroid cancer in a subject which comprises detecting the presence of antibodies capable of immunospecific binding to ROR1, or one or more epitope-containing fragments thereof in said subject, in which (a) the presence of an elevated level of antibodies capable of immunospecific binding to ROR1 or said one or more epitope-containing fragments thereof in said subject as compared with the level in a healthy subject or (b) the presence of a detectable level of antibodies capable of immunospecific binding to ROR1 or said one or more epitope-containing fragments thereof in said subject as compared with a corresponding undetectable level in a healthy subject indicates the presence of said cancer in said subject.

One particular method of detecting, diagnosing and/or screening for cancer, e.g. bladder cancer, breast cancer, colorectal cancer, head and neck cancer, kidney cancer, liver cancer, lung cancer, ovarian cancer, pancreatic cancer, skin cancer or thyroid cancer comprises:

bringing into contact with a biological sample to be tested ROR1, or one or more epitope-containing fragments thereof; and

detecting the presence of antibodies in the subject capable of immunospecific binding to ROR1, or one or more epitope-containing fragments thereof.

According to another aspect of the invention there is provided a method of monitoring the progression of cancer, e.g. bladder cancer, breast cancer, colorectal cancer, head and neck cancer, kidney cancer, liver cancer, lung cancer, ovarian cancer, pancreatic cancer, skin cancer or thyroid cancer or of monitoring the effect of an anti-cancer e.g. anti-bladder cancer, anti-breast cancer, anti-colorectal cancer, anti-head and neck cancer, anti-kidney cancer, anti-liver cancer, anti-lung cancer, anti-ovarian cancer, anti-pancreatic cancer, anti-skin cancer or anti-thyroid cancer drug or therapy in a subject which comprises detecting the presence of antibodies capable of immunospecific binding to ROR1, or one or more epitope-containing fragments thereof in said subject at a first time point and at a later time point, the presence of an elevated or lowered level of antibodies capable of immunospecific binding to ROR1, or one or more epitope-containing fragments thereof in said subject at the later time point as compared with the level in said subject at said first time point, indicating the progression or regression of said cancer, or the effect or non-effect of said anti-cancer drug or therapy in said subject.

The presence of antibodies capable of immunospecific binding to ROR1, or one or more epitope-containing fragments thereof is typically detected by analysis of a biological sample obtained from said subject (exemplary biological samples are mentioned above, e.g. the sample is a sample of bladder, breast, colorectal, head and neck, kidney, liver, lung, ovarian, pancreatic, skin or thyroid tissue, or else a sample of blood or saliva). The method typically includes the step of obtaining said biological sample for analysis from said subject. The antibodies that may be detected include IgA, IgM and IgG antibodies.

In any of the above methods, the level that may be detected in the candidate subject who has cancer, e.g. bladder cancer, breast cancer, colorectal cancer, head and neck cancer, kidney cancer, liver cancer, lung cancer, ovarian cancer, pancreatic cancer, skin cancer or thyroid cancer is 2 or more fold higher than the level in the healthy subject.

In one aspect of the invention, one-dimensional electrophoresis or other appropriate methods are used to analyze bladder cancer, breast cancer, colorectal cancer, head and neck cancer, liver cancer, lung cancer, ovarian cancer, pancreatic cancer, skin cancer or thyroid cancer tissue samples from a subject, preferably a living subject, in order to measure the expression of the protein of the invention for screening or diagnosis of e.g. bladder cancer, breast cancer, colorectal cancer, head and neck cancer, kidney cancer, liver cancer, lung cancer, ovarian cancer, pancreatic cancer, skin cancer or thyroid cancer, to determine the prognosis of a bladder cancer, breast cancer, colorectal cancer, head and neck cancer, liver cancer, lung cancer, ovarian cancer, pancreatic cancer, skin cancer or thyroid cancer patient, to monitor the effectiveness of bladder cancer, breast cancer, colorectal cancer, head and neck cancer, liver cancer, lung cancer, ovarian cancer, pancreatic cancer, skin cancer or thyroid cancer therapy, or for drug development.

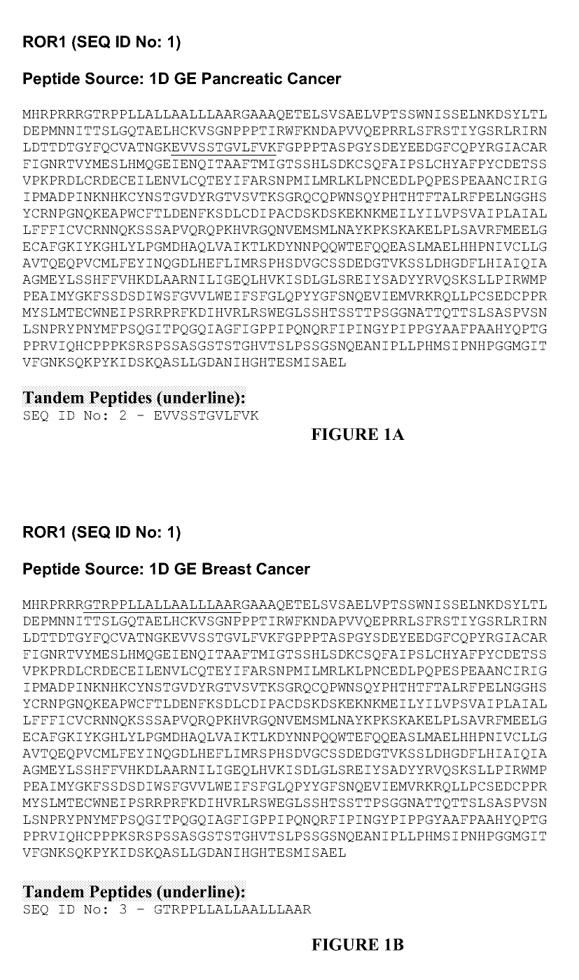

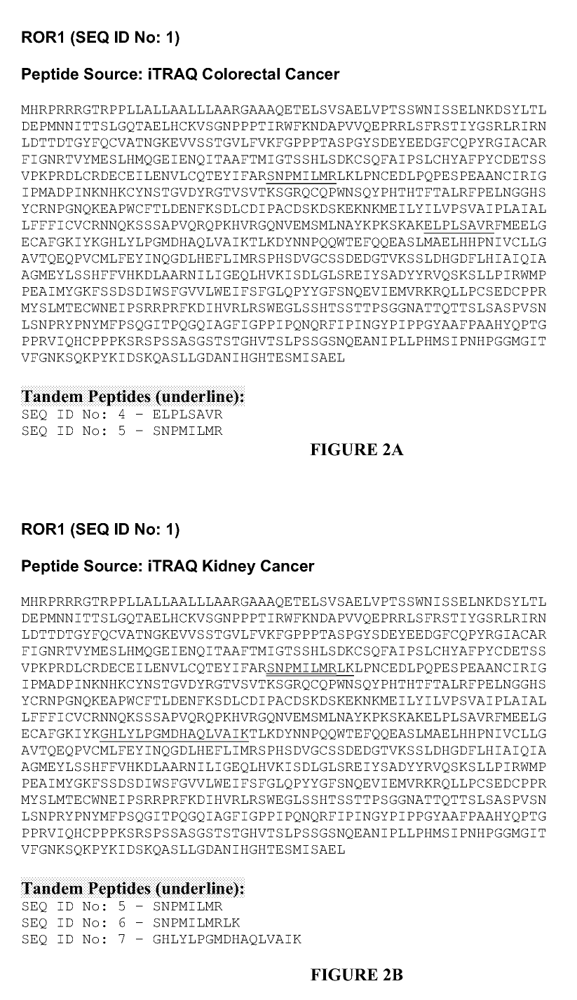

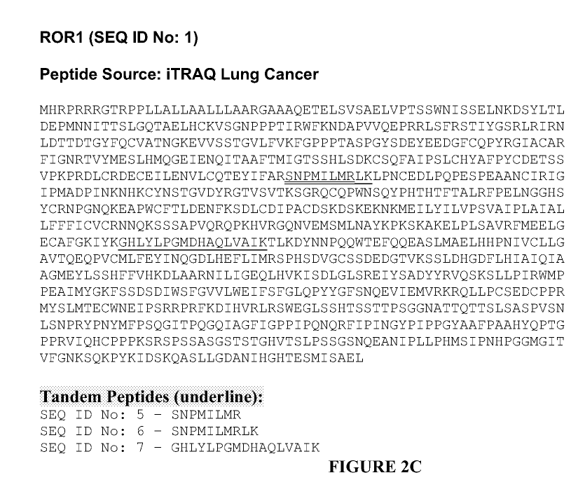

As used herein, the term “Protein of the invention”, or “ROR1”, refers to the protein illustrated as the human long variant in FIGS. 1 and 2 detected experimentally by 1D gel electrophoresis of cancer tissue samples. Protein derivatives of this sequence may also be useful for the same purposes as described herein.

This protein has been identified in membrane protein extracts of cancer tissue samples from cancer patients, through the methods and apparatus of the Preferred Technology described in Examples 1 and 2 (e.g. 1D gel electrophoresis and tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at www.expasy.com), and the following entry: Q01973, Tyrosine-protein kinase transmembrane receptor ROR1 was identified. The nucleotide sequence encoding this protein is found at accession number NM_{—}005012, which is expressly incorporated herein by reference.

According to SWISS-PROT, Tyrosine-protein kinase transmembrane receptor ROR1 is expressed strongly in human heart, lung, and kidney, but weakly in the CNS. The short isoform (missing amino acids 1-549 of SEQ ID No: 1) is strongly expressed in fetal and adult CNS and in a variety of human cancers, including those originating from CNS or PNS neuroectoderm. Tyrosine-protein kinase transmembrane receptor ROR1 is expressed at high levels during early embryonic development. The expression levels drop strongly around day 16 and there are only very low levels in adult tissues.

Immunohistochemistry experiments (see Example 3) showed strong staining in bladder cancer, breast cancer, colorectal cancer, head and neck cancer, kidney cancer, liver cancer, lung cancer, ovarian cancer, pancreatic cancer, skin cancer and thyroid cancer.

The protein of the invention is useful as are fragments particularly epitope containing fragments e.g. antigenic or immunogenic fragments thereof and derivatives thereof. Epitope containing fragments including antigenic or immunogenic fragments will typically be of length 12 amino acids or more e.g. 20 amino acids or more e.g. 50 or 100 amino acids or more. Fragments may be 95% or more of the length of the full protein e.g. 90% or more e.g. 75% or 50% or 25% or 10% or more of the length of the full protein.

Alternatively, the protein/polypeptide employed or referred to herein may be limited to those specifically recited/described in the present specification or a moiety 80, 85, 90, 91, 92, 93, 94, 95, 96, 97, 98 or 99% identical or similar thereto.

Epitope containing fragments including antigenic or immunogenic fragments will be capable of eliciting a relevant immune response in a patient. DNA encoding the protein of the invention is also useful as are fragments thereof e.g. DNA encoding fragments of the protein of the invention such as immunogenic fragments thereof. Fragments of nucleic acid (e.g. DNA) encoding the protein of the invention may be 95% or more of the length of the full coding region e.g. 90% or more e.g. 75% or 50% or 25% or 10% or more of the length of the full coding region. Fragments of nucleic acid (e.g. DNA) may be 36 nucleotides or more e.g. 60 nucleotides or more e.g. 150 or 300 nucleotides or more in length.

Derivatives of the protein of the invention include variants on the sequence in which one or more (e.g. 1-20 such as 15 amino acids, or up to 20% such as up to 10% or 5% or 1% by number of amino acids based on the total length of the protein) deletions, insertions or substitutions have been made. Substitutions may typically be conservative substitutions. Derivatives will typically have essentially the same biological function as the protein from which they are derived. Derivatives will typically be comparably antigenic or immunogenic to the protein from which they are derived. Derivatives will typically have either the ligand-binding activity, or the active receptor-complex forming ability, or preferably both, of the protein from which they are derived.

Derivatives of proteins also include chemically treated protein such as carboxymethylated, carboxyamidated, acetylated proteins, for example treated during purification.

For ROR1, the detected level obtained upon analyzing tissue from subjects having e.g. bladder cancer, breast cancer, colorectal cancer, head and neck cancer, kidney cancer, liver cancer, lung cancer, ovarian cancer, pancreatic cancer, skin cancer or thyroid cancer relative to the detected level obtained upon analyzing tissue from subjects free from e.g. bladder cancer, breast cancer, colorectal cancer, head and neck cancer, kidney liver cancer, lung cancer, ovarian cancer, pancreatic cancer, skin cancer and thyroid cancer will depend upon the particular analytical protocol and detection technique that is used. Accordingly, the present invention contemplates that each laboratory will establish a reference range in subjects free from e.g. bladder cancer, breast cancer, colorectal cancer, head and neck cancer, kidney cancer, liver cancer, lung cancer, ovarian cancer, pancreatic cancer, skin cancer and thyroid cancer according to the analytical protocol and detection technique in use, as is conventional in the diagnostic art. Preferably, at least one control positive tissue sample from a subject known to have e.g. bladder cancer, breast cancer, colorectal cancer, head and neck cancer, liver cancer, lung cancer, ovarian cancer, pancreatic cancer, skin cancer or thyroid cancer or at least one control negative tissue sample from a subject known to be free from e.g. bladder cancer, breast cancer, colorectal cancer, head and neck cancer, liver cancer, lung cancer, ovarian cancer, pancreatic cancer, skin cancer and thyroid cancer (and more preferably both positive and negative control samples) are included in each batch of test samples analysed.

ROR1 can be used for detection, prognosis, diagnosis, or monitoring of e.g. bladder cancer, breast cancer, colorectal cancer, head and neck cancer, kidney cancer, liver cancer, lung cancer, ovarian cancer, pancreatic cancer, skin cancer or thyroid cancer or for drug development. In one embodiment of the invention, tissue from a subject (e.g. a subject suspected of having bladder cancer, breast cancer, colorectal cancer, head and neck cancer, kidney cancer, liver cancer, lung cancer, ovarian cancer, pancreatic cancer, skin cancer or thyroid cancer) is analysed by 1D electrophoresis for detection of ROR1. An increased abundance of ROR1 in the tissue from the subject relative to tissue from a subject or subjects free from bladder cancer, breast cancer, colorectal cancer, head and neck cancer, kidney cancer, liver cancer, lung cancer, ovarian cancer, pancreatic cancer, skin cancer and thyroid cancer (e.g. a control sample) or a previously determined reference range indicates the presence of bladder cancer, breast cancer, colorectal cancer, head and neck cancer, kidney cancer, liver cancer, lung cancer, ovarian cancer, pancreatic cancer, skin cancer or thyroid cancer.

ROR1 may, in particular, be characterized as an isoform having a MW substantially as recited defined in SEQ ID NO: 1 (e.g. +/−10%, particularly +/−5% of the value).

In relation to fragments, epitope containing fragments, immunogenic fragments or antigenic fragments of ROR1:

for the relevant cancer applications, in one aspect of the invention these comprise the sequences identified as tryptic sequences in Examples 1 and 2.

As used herein, ROR1 is “isolated” when it is present in a preparation that is substantially free of contaminating proteins, i.e. a preparation in which less than 10% (for example less than 5%, such as less than 1%) of the total protein present is contaminating protein(s). A contaminating protein is a protein having a significantly different amino acid sequence from that of isolated ROR1, as determined by mass spectral analysis. As used herein, a “significantly different” sequence is one that permits the contaminating protein to be resolved from ROR1 by mass spectral analysis, performed according to the Reference Protocol described herein in Example 1.

Thus in one aspect the invention provides a pharmaceutical composition for the treatment of e.g. bladder cancer, breast cancer, colorectal cancer, head and neck cancer, kidney cancer, liver cancer, lung cancer, ovarian cancer, pancreatic cancer, skin cancer and/or thyroid cancer comprising a therapeutically effective amount of a ROR1 polypeptide (particularly those defined above) or an immunogenic fragment thereof and an adjuvant.

ROR1 can be assayed by any method known to those skilled in the art, including but not limited to, the Preferred Technologies described herein, kinase assays, enzyme assays, binding assays and other functional assays, immunoassays, and western blotting. In one embodiment, ROR1 is separated on a 1-D gel by virtue of its MW and visualized by staining the gel. In one embodiment, ROR1 is stained with a fluorescent dye and imaged with a fluorescence scanner. Sypro Red (Molecular Probes, Inc., Eugene, Oreg.) is a suitable dye for this purpose. A preferred fluorescent dye is disclosed in U.S. Pat. No. 6,335,446, filed on Oct. 5, 1999, which is incorporated herein by reference in its entirety.

Alternatively, ROR1 can be detected in an immunoassay. In one embodiment, an immunoassay is performed by contacting a sample from a subject to be tested with an anti-ROR1 antibody (or other affinity reagent) under conditions such that binding (e.g. immunospecific binding) can occur if ROR1 is present, and detecting or measuring the amount of any binding (e.g. immunospecific binding) by the agent. ROR1 binding agents can be produced by the methods and techniques taught herein. In a particular embodiment, ROR1 is analysed using immunohistochemistry.

ROR1 may be detected by virtue of the detection of a fragment thereof e.g. an epitope containing (e.g. an immunogenic or antigenic) fragment thereof. Fragments may have a length of at least 10, more typically at least 20 amino acids e.g. at least 50 or 100 amino acids e.g. at least 150 or 200 amino acids; e.g. at least 300 or 500 amino acids; e.g. at least 700 or 900 amino acids.

In one embodiment, binding of an affinity reagent (e.g. an antibody) in tissue sections can be used to detect aberrant ROR1 localization or an aberrant level of ROR1. In a specific embodiment, an antibody (or other affinity reagent) to ROR1 can be used to assay a patient tissue (e.g. a bladder, breast, colorectal, head and neck, kidney, liver, lung, ovarian, pancreatic, skin or thyroid tissue) for the level of ROR1 where an aberrant level of ROR1 is indicative of bladder cancer, breast cancer, colorectal cancer, head and neck cancer, kidney cancer, liver cancer, lung cancer, ovarian cancer, pancreatic cancer, skin cancer or thyroid cancer. As used herein, an “aberrant level” means a level that is increased compared with the level in a subject free from bladder cancer, breast cancer, colorectal cancer, head and neck cancer, kidney cancer, liver cancer, lung cancer, ovarian cancer, pancreatic cancer, skin cancer and thyroid cancer or a reference level.

Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, ROR1 can be detected in a fluid sample (e.g. blood, urine, or saliva) by means of a two-step sandwich assay. In the first step, a capture reagent (e.g. an anti-ROR1 antibody or other affinity reagent) is used to capture ROR1. The capture reagent can optionally be immobilized on a solid phase. In the second step, a directly or indirectly labeled detection reagent is used to detect the captured ROR1. In one embodiment, the detection reagent is a lectin. Any lectin can be used for this purpose that preferentially binds to ROR1 rather than to other isoforms that have the same core protein as ROR1 or to other proteins that share the antigenic determinant recognized by the antibody. In a preferred embodiment, the chosen lectin binds ROR1 with at least 2-fold greater affinity, more preferably at least 5-fold greater affinity, still more preferably at least 10-fold greater affinity, than to said other isoforms that have the same core protein as ROR1 or to said other proteins that share the antigenic determinant recognized by the affinity reagent. Based on the present description, a lectin that is suitable for detecting ROR1 can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety). In an alternative embodiment, the detection reagent is an antibody (or other affinity reagent), e.g. an antibody that specifically (e.g. immunospecifically) detects other post-translational modifications, such as an antibody that immunospecifically binds to phosphorylated amino acids. Examples of such antibodies include those that bind to phosphotyrosine (BD Transduction Laboratories, catalog nos.: P11230-050/P11230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalogue nos. 71-8200, 13-9200).

If desired, a gene encoding ROR1, a related gene, or related nucleic acid sequences or subsequences, including complementary sequences, can also be used in hybridization assays. A nucleotide encoding ROR1, or subsequences thereof comprising at least 8 nucleotides, preferably at least 12 nucleotides, and most preferably at least 15 nucleotides can be used as a hybridization probe. Hybridization assays can be used for detection, prognosis, diagnosis, or monitoring of conditions, disorders, or disease states, associated with aberrant expression of the gene encoding ROR1, or for differential diagnosis of subjects with signs or symptoms suggestive of e.g. bladder cancer, breast cancer, colorectal cancer, head and neck cancer, kidney cancer, liver cancer, lung cancer, ovarian cancer, pancreatic cancer, skin cancer or thyroid cancer. In particular, such a hybridization assay can be carried out by a method comprising contacting a subject's sample containing nucleic acid with a nucleic acid probe capable of hybridizing to a DNA or RNA that encodes ROR1, under conditions such that hybridization can occur, and detecting or measuring any resulting hybridization.

Hence nucleic acid encoding ROR1 (e.g. DNA or more suitably RNA) may be detected, for example, using a hybridizing agent capable of hybridizing to nucleic acid encoding ROR1.

One such exemplary method comprises:

contacting one or more oligonucleotide probes comprising 10 or more consecutive nucleotides complementary to a nucleotide sequence encoding ROR1, with an RNA obtained from a biological sample from the subject or with cDNA copied from the RNA, wherein said contacting occurs under conditions that permit hybridization of the probe to the nucleotide sequence if present;

detecting hybridization, if any, between the probe and the nucleotide sequence; and

comparing the hybridization, if any, detected in step (b) with the hybridization detected in a control sample, or with a previously determined reference range.

The invention also provides diagnostic kits, comprising an anti-ROR1 antibody (or other affinity reagent). In addition, such a kit may optionally comprise one or more of the following: (1) instructions for using the anti-ROR1 affinity reagent for diagnosis, prognosis, therapeutic monitoring or any combination of these applications; (2) a labeled binding partner to the affinity reagent; (3) a solid phase (such as a reagent strip) upon which the anti-ROR1 affinity reagent is immobilized; and (4) a label or insert indicating regulatory approval for diagnostic, prognostic or therapeutic use or any combination thereof. If no labeled binding partner to the affinity reagent is provided, the anti-ROR1 affinity reagent itself can be labeled with a detectable marker, e.g. a chemiluminescent, enzymatic, fluorescent, or radioactive moiety.

The invention also provides a kit comprising a nucleic acid probe capable of hybridizing to nucleic acid, suitably RNA, encoding ROR1. In a specific embodiment, a kit comprises one or more containers a pair of primers (e.g. each in the size range of 6-30 nucleotides, more preferably 10-30 nucleotides and still more preferably 10-20 nucleotides) that under appropriate reaction conditions can prime amplification of at least a portion of a nucleic acid encoding ROR1, such as by polymerase chain reaction (see, e.g. Innis et al., 1990, PCR Protocols, Academic Press, Inc., San Diego, Calif.), ligase chain reaction (see EP 320,308) use of Qβ replicase, cyclic probe reaction, or other methods known in the art.

A kit can optionally further comprise a predetermined amount of ROR1 or a nucleic acid encoding ROR1, e.g. for use as a standard or control.

The biological sample used can be from any source such as a serum sample or a tissue sample e.g. bladder, breast, colorectal, head and neck, kidney, liver, lung, ovarian, pancreatic, skin or thyroid tissue. For instance, when looking for evidence of metastatic bladder cancer, breast cancer, colorectal cancer, head and neck cancer, kidney cancer, liver cancer, lung cancer, ovarian cancer, pancreatic cancer, skin cancer or thyroid cancer, one would look at major sites of bladder cancer, breast cancer, colorectal cancer, head and neck cancer, liver cancer, lung cancer, ovarian cancer, pancreatic cancer, skin cancer or thyroid cancer metastasis, e.g. the prostate, uterus, vagina, bones, liver or lungs for bladder cancer; the liver, lungs and bones for breast cancer; the liver, peritoneal cavity, pelvis, retroperitoneum and lungs for colorectal cancer; the lungs, bones and liver for head and neck cancer; the lungs and bones for liver cancer; the brain, liver, bones and adrenal glands for lung cancer; the abdomen for ovarian cancer; the liver for pancreatic cancer; the lungs, brain and bones for skin cancer and the lungs and bones for thyroid cancer.

Alternatively the presence of ROR1, or one or more fragments thereof, or the presence of nucleic acid encoding ROR1 or the presence of the activity of ROR1 may be detected by analysis in situ.

In certain embodiments, methods of diagnosis described herein may be at least partly, or wholly, performed in vitro.

Suitably the presence of ROR1, or one or more fragments thereof, or the presence of nucleic acid encoding ROR1 or the presence of the activity of ROR1 is detected quantitatively.

For example, quantitatively detecting may comprise:

contacting a biological sample with an affinity reagent that is specific for ROR1, said affinity reagent optionally being conjugated to a detectable label; and

detecting whether binding has occurred between the affinity reagent and at least one species in the sample, said detection being performed either directly or indirectly.

Alternatively the presence of ROR1, or one or more fragments thereof, or the presence of nucleic acid encoding ROR1 or the presence of the activity of ROR1 may be detected quantitatively by means involving use of an imaging technology.

In another embodiment, the method of the invention involves use of immunohistochemistry on e.g. bladder, breast, colorectal, head and neck, kidney, liver, lung, ovarian, pancreatic, skin or thyroid tissue sections in order to determine the presence of ROR1, or one or more fragments thereof, or the presence of nucleic acid encoding ROR1 or the presence of the activity of ROR1, and thereby to localise e.g. bladder cancer, breast cancer, colorectal cancer, head and neck cancer, kidney cancer, liver cancer, lung cancer, ovarian cancer, pancreatic cancer, skin cancer or thyroid cancer cells.

In one embodiment the presence of ROR1 or one or more epitope-containing fragments thereof is detected, for example using an affinity reagent capable of specific binding to ROR1 or one or more fragments thereof, such as an antibody.

In another embodiment the activity of ROR1 is detected. ROR1 belongs to the small ROR subfamily of RTKs, which are involved in diverse developmental functions such as neurite growth and branching, and heart and skeletal formation. At the cellular level they are involved in cell migration and planar cell polarity, influencing asymmetric cell division in early development (Oncol Rep. 2005 14(6):1583-8). Wnt5a is a candidate ligand of ROR1, stimulating activation of NFkappaB in proportion to the level of expression of the receptor. This signaling activity has been independently verified in CHO cells, and shown to promote survival of chronic lymphocytic leukemia (CLL) cells in culture (PNAS 2008, 105(8): 3047-3052).

Use in Clinical Studies

The diagnostic methods and compositions of the present invention can assist in monitoring a clinical study, e.g. to evaluate drugs for therapy of bladder cancer, breast cancer, colorectal cancer, head and neck cancer, kidney cancer, liver cancer, lung cancer, ovarian cancer, pancreatic cancer, skin cancer or thyroid cancer. In one embodiment, candidate molecules are tested for their ability to restore ROR1 levels in a subject having e.g. bladder cancer, breast cancer, colorectal cancer, head and neck cancer, kidney cancer, liver cancer, lung cancer, ovarian cancer, pancreatic cancer, skin cancer or thyroid cancer to levels found in subjects free from bladder cancer, breast cancer, colorectal cancer, head and neck cancer, kidney cancer, liver cancer, lung cancer, ovarian cancer, pancreatic cancer, skin cancer and thyroid cancer or, in a treated subject, to preserve ROR1 levels at or near non-bladder cancer, non-breast cancer, non-colorectal cancer, non-head and neck cancer, non-kidney cancer, non-liver cancer, non-lung cancer, non-ovarian cancer, non-pancreatic cancer, non-skin cancer or non-thyroid cancer values.

In another embodiment, the methods and compositions of the present invention are used to screen candidates for a clinical study to identify individuals having e.g. bladder cancer, breast cancer, colorectal cancer, head and neck cancer, kidney cancer, liver cancer, lung cancer, ovarian cancer, pancreatic cancer, skin cancer or thyroid cancer; such individuals can then be excluded from the study or can be placed in a separate cohort for treatment or analysis.

Production of Protein of the Invention and Corresponding Nucleic Acid

In one aspect the invention provides a method of treating or preventing e.g. bladder cancer, breast cancer, colorectal cancer, head and neck cancer, kidney cancer, liver cancer, lung cancer, ovarian cancer, pancreatic cancer, skin cancer and/or thyroid cancer, comprising administering to a subject in need of such treatment or prevention a therapeutically effective amount of nucleic acid encoding ROR1 or one or more fragments or derivatives thereof, for example in the form of a vaccine.

In another aspect there is provided a method of treating or preventing e.g. bladder cancer, breast cancer, colorectal cancer, head and neck cancer, kidney cancer, liver cancer, lung cancer, ovarian cancer, pancreatic cancer, skin cancer and/or thyroid cancer comprising administering to a subject in need of such treatment or prevention a therapeutically effective amount of nucleic acid that inhibits the function or expression of ROR1.

The methods (and/or other DNA aspects disclosed herein) of the invention may, for example include wherein the nucleic acid is a ROR1 anti-sense nucleic acid or ribozyme.

Thus the invention includes the use of nucleic acid encoding ROR1 or one or more fragments or derivatives thereof, in the manufacture of a medicament for treating or preventing e.g. bladder cancer, breast cancer, colorectal cancer, head and neck cancer, kidney cancer, liver cancer, lung cancer, ovarian cancer, pancreatic cancer, skin cancer and/or thyroid cancer.

There is also provided the use of nucleic acid that inhibits the function or expression of ROR1 in the manufacture of a medicament for treating or preventing e.g. bladder cancer, breast cancer, colorectal cancer, head and neck cancer, kidney cancer, liver cancer, lung cancer, ovarian cancer, pancreatic cancer, skin cancer and/or thyroid cancer.

A DNA employed in the present invention can be obtained by isolation as a cDNA fragment from cDNA libraries using as starter materials commercial mRNAs and determining and identifying the nucleotide sequences thereof. That is, specifically, clones are randomly isolated from cDNA libraries, which are prepared according to Ohara et al.'s method (DNA Research Vol. 4, 53-59 (1997)). Next, through hybridization, duplicated clones (which appear repeatedly) are removed and then in vitro transcription and translation are carried out. Nucleotide sequences of both termini of clones, for which products of 50 kDa or more are confirmed, are determined.

Furthermore, databases of known genes are searched for homology using the thus obtained terminal nucleotide sequences as queries.

In addition to the above screening method, the 5′ and 3′ terminal sequences of cDNA are related to a human genome sequence. Then an unknown long-chain gene is confirmed in a region between the sequences, and the full-length of the cDNA is analyzed. In this way, an unknown gene that is unable to be obtained by a conventional cloning method that depends on known genes can be systematically cloned.

Moreover, all of the regions of a human-derived gene containing a DNA of the present invention can also be prepared using a PCR method such as RACE while paying sufficient attention to prevent artificial errors from taking place in short fragments or obtained sequences. As described above, clones having DNA of the present invention can be obtained.

In another means for cloning DNA of the present invention, a synthetic DNA primer having an appropriate nucleotide sequence of a portion of a polypeptide of the present invention is produced, followed by amplification by the PCR method using an appropriate library. Alternatively, selection can be carried out by hybridization of the DNA of the present invention with a DNA that has been incorporated into an appropriate vector and labeled with a DNA fragment or a synthetic DNA encoding some or all of the regions of the polypeptide of the present invention. Hybridization can be carried out by, for example, the method described in Current Protocols in Molecular Biology (edited by Frederick M. Ausubel et al., 1987). DNA of the present invention may be any DNA, as long as they contain nucleotide sequences encoding the polypeptides of the present invention as described above. Such a DNA may be a cDNA identified and isolated from cDNA libraries or the like that are derived from bladder, breast, colorectal, head and neck, kidney, liver, lung, ovarian, pancreatic, skin or thyroid tissue. Such a DNA may also be a synthetic DNA or the like. Vectors for use in library construction may be any of bacteriophages, plasmids, cosmids, phargemids, or the like. Furthermore, by the use of a total RNA fraction or a mRNA fraction prepared from the above cells and/or tissues, amplification can be carried out by a direct reverse transcription coupled polymerase chain reaction (hereinafter abbreviated as “RT-PCR method”).

DNA encoding the above polypeptide consisting of an amino acid sequence that is substantially identical to the amino acid sequence of ROR1 or DNA encoding the above polypeptide consisting of an amino acid sequence derived from the amino acid sequence of ROR1 by deletion, substitution, or addition of one or more amino acids composing a portion of the amino acid sequence can be easily produced by an appropriate combination of, for example, a site-directed mutagenesis method, a gene homologous recombination method, a primer elongation method, and the PCR method known by persons skilled in the art. In addition, at this time, a possible method for causing a polypeptide to have substantially equivalent biological activity is substitution of homologous amino acids (e.g. polar and nonpolar amino acids, hydrophobic and hydrophilic amino acids, positively-charged and negatively charged amino acids, and aromatic amino acids) among amino acids composing the polypeptide. Furthermore, to maintain substantially equivalent biological activity, amino acids within functional domains contained in the polypeptide of the present invention are preferably conserved.

Furthermore, examples of DNA of the present invention include DNA comprising a nucleotide sequence that encodes the amino acid sequence of ROR1 and DNA hybridizing under stringent conditions to the DNA and encoding a polypeptide (protein) having biological activity (function) equivalent to the function of the polypeptide consisting of the amino acid sequence of ROR1. Under such conditions, an example of such DNA capable of hybridizing to DNA comprising the nucleotide sequence that encodes the amino acid sequence of ROR1 is DNA comprising a nucleotide sequence that has a degree of overall mean homology with the entire nucleotide sequence of the DNA, such as approximately 80% or more, preferably approximately 90% or more, and more preferably approximately 95% or more. Hybridization can be carried out according to a method known in the art such as a method described in Current Protocols in Molecular Biology (edited by Frederick M. Ausubel et al., 1987) or a method according thereto. Here, “stringent conditions” are, for example, conditions of approximately “1*SSC, 0.1% SDS, and 37° C., more stringent conditions of approximately “0.5*SSC, 0.1% SDS, and 42° C., or even more stringent conditions of approximately “0.2*SSC, 0.1% SDS, and 65° C. With more stringent hybridization conditions, the isolation of a DNA having high homology with a probe sequence can be expected. The above combinations of SSC, SDS, and temperature conditions are given for illustrative purposes. Stringency similar to the above can be achieved by persons skilled in the art using an appropriate combination of the above factors or other factors (for example, probe concentration, probe length, and reaction time for hybridization) for determination of hybridization stringency.

A cloned DNA of the present invention can be directly used or used, if desired, after digestion with a restriction enzyme or addition of a linker, depending on purposes. The DNA may have ATG as a translation initiation codon at the 5′ terminal side and have TAA, TGA, or TAG as a translation termination codon at the 3′ terminal side. These translation initiation and translation termination codons can also be added using an appropriate synthetic DNA adapter.

In the methods/uses of the invention, ROR1 may for example be provided in isolated form, such as where the ROR1 polypeptide has been purified to at least to some extent. ROR1 polypeptide may be provided in substantially pure form, that is to say free, to a substantial extent, from other proteins. ROR1 polypeptide can also be produced using recombinant methods, synthetically produced or produced by a combination of these methods. ROR1 can be easily prepared by any method known by persons skilled in the art, which involves producing an expression vector containing appropriate DNA of the present invention or a gene containing a DNA of the present invention, culturing a transformant transformed using the expression vector, generating and accumulating a relevant polypeptide of the present invention or a recombinant protein containing the polypeptide, and then collecting the resultant.

Recombinant ROR1 polypeptide may be prepared by processes well known in the art from genetically engineered host cells comprising expression systems. Accordingly, the present invention also relates to expression systems which comprise a ROR1 polypeptide or nucleic acid, to host cells which are genetically engineered with such expression systems and to the production of ROR1 polypeptide by recombinant techniques. For recombinant ROR1 polypeptide production, host cells can be genetically engineered to incorporate expression systems or portions thereof for nucleic acids. Such incorporation can be performed using methods well known in the art, such as, calcium phosphate transfection, DEAD-dextran mediated transfection, transvection, microinjection, cationic lipid-mediated transfection, electroporation, transduction, scrape loading, ballistic introduction or infection (see e.g. Davis et al., Basic Methods in Molecular Biology, 1986 and Sambrook et al., Molecular Cloning: A Laboratory Manual, 2nd Ed., Cold Spring Harbour laboratory Press, Cold Spring Harbour, NY, 1989).

As host cells, for example, bacteria of the genus Escherichia, Streptococci, Staphylococci, Streptomyces, bacteria of the genus Bacillus, yeast, Aspergillus cells, insect cells, insects, and animal cells are used. Specific examples of bacteria of the genus Escherichia, which are used herein, include Escherichia coli K12 and DH1 (Proc. Natl. Acad. Sci. U.S.A., Vol. 60, 160 (1968)), JM103 (Nucleic Acids Research, Vol. 9, 309 (1981)), JA221 (Journal of Molecular Biology, Vol. 120, 517 (1978)), and HB101 (Journal of Molecular Biology, Vol. 41, 459 (1969)). As bacteria of the genus Bacillus, for example, Bacillus subtilis MI114 (Gene, Vol. 24, 255 (1983)) and 207-21 (Journal of Biochemistry, Vol. 95, 87 (1984)) are used. As yeast, for example, Saccaromyces cerevisiae AH22, AH22R-, NA87-11A, DKD-5D, and 20B-12, Schizosaccaromyces pombe NCYC1913 and NCYC2036, and Pichia pastoris are used. As insect cells, for example, Drosophila S2 and Spodoptera Sf9 cells are used. As animal cells, for example, COS-7 and Vero monkey cells, CHO Chinese hamster cells (hereinafter abbreviated as CHO cells), dhfr-gene-deficient CHO cells, mouse L cells, mouse AtT-20 cells, mouse myeloma cells, rat GH3 cells, human FL cells, COS, HeLa, C127,3T3, HEK 293, BHK and Bowes melanoma cells are used.

Cell-free translation systems can also be employed to produce recombinant polypeptides (e.g. rabbit reticulocyte lysate, wheat germ lysate, SP6/T7 in vitro T&T and RTS100 E. Coli HY transcription and translation kits from Roche Diagnostics Ltd., Lewes, UK and the TNT Quick coupled Transcription/Translation System from Promega UK, Southampton, UK).

The expression vector can be produced according to a method known in the art. For example, the vector can be produced by (1) excising a DNA fragment containing a DNA of the present invention or a gene containing a DNA of the present invention and (2) ligating the DNA fragment downstream of the promoter in an appropriate expression vector. A wide variety of expression systems can be used, such as and without limitation, chromosomal, episomal and virus-derived systems, e.g. plasmids derived from Escherichia coli (e.g. pBR322, pBR325, pUC18, and pUC118), plasmids derived from Bacillus subtilis (e.g. pUB 110, pTP5, and pC194), from bacteriophage, from transposons, from yeast episomes (e.g. pSH19 and pSH15), from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage (such as [lambda] phage) genetic elements, such as cosmids and phagemids. The expression systems may contain control regions that regulate as well as engender expression. Promoters to be used in the present invention may be any promoters as long as they are appropriate for hosts to be used for gene expression. For example, when a host is Escherichia coli, a trp promoter, a lac promoter, a recA promoter, a pL promoter, an 1 pp promoter, and the like are preferred. When a host is Bacillus subtilis, an SPO1 promoter, an SPO2 promoter, a penP promoter, and the like are preferred. When a host is yeast, a PHOS promoter, a PGK promoter, a GAP promoter, an ADH promoter, and the like are preferred. When an animal cell is used as a host, examples of promoters for use in this case include an SRa promoter, an SV40 promoter, an LTR promoter, a CMV promoter, and an HSV-TK promoter. Generally, any system or vector that is able to maintain, propagate or express a nucleic acid to produce a polypeptide in a host may be used.

The appropriate nucleic acid sequence may be inserted into an expression system by any variety of well known and routine techniques, such as those set forth in Sambrook et al., supra. Appropriate secretion signals may be incorporated into the ROR1 polypeptide to allow secretion of the translated protein into the lumen of the endoplasmic reticulum, the periplasmic space or the extracellular environment. These signals may be endogenous to the ROR1 polypeptide or they may be heterologous signals. Transformation of the host cells can be carried out according to methods known in the art. For example, the following documents can be referred to: Proc. Natl. Acad. Sci. U.S.A., Vol. 69, 2110 (1972); Gene, Vol. 17, 107 (1982); Molecular & General Genetics, Vol. 168, 111 (1979); Methods in Enzymology, Vol. 194, 182-187 (1991); Proc. Natl. Acad. Sci. U.S.A.), Vol. 75, 1929 (1978); Cell Technology, separate volume 8, New Cell Technology, Experimental Protocol. 263-267 (1995) (issued by Shujunsha); and Virology, Vol. 52, 456 (1973). The thus obtained transformant transformed with an expression vector containing a DNA of the present invention or a gene containing a DNA of the present invention can be cultured according to a method known in the art. For example, when hosts are bacteria of the genus Escherichia, the bacteria are generally cultured at approximately 15° C. to 43° C. for approximately 3 to 24 hours. If necessary, aeration or agitation can also be added. When hosts are bacteria of the genus Bacillus, the bacteria are generally cultured at approximately 30° C. to 40° C. for approximately 6 to 24 hours. If necessary, aeration or agitation can also be added. When transformants whose hosts are yeast are cultured, culture is generally carried out at approximately 20° C. to 35° C. for approximately 24 to 72 hours using media with pH adjusted to be approximately 5 to 8. If necessary, aeration or agitation can also be added. When transformants whose hosts are animal cells are cultured, the cells are generally cultured at approximately 30° C. to 40° C. for approximately 15 to 60 hours using media with the pH adjusted to be approximately 6 to 8. If necessary, aeration or agitation can also be added.

If a ROR1 polypeptide is to be expressed for use in cell-based screening assays, it is preferred that the polypeptide be produced at the cell surface. In this event, the cells may be harvested prior to use in the screening assay. If the ROR1 polypeptide is secreted into the medium, the medium can be recovered in order to isolate said polypeptide. If produced intracellularly, the cells must first be lysed before the ROR1 polypeptide is recovered.

ROR1 polypeptide can be recovered and purified from recombinant cell cultures or from other biological sources by well known methods including, ammonium sulphate or ethanol precipitation, acid extraction, anion or cation exchange chromatography, phosphocellulose chromatography, affinity chromatography, hydrophobic interaction chromatography, hydroxylapatite chromatography, molecular sieving chromatography, centrifugation methods, electrophoresis methods and lectin chromatography. In one embodiment, a combination of these methods is used. In another embodiment, high performance liquid chromatography is used. In a further embodiment, an antibody which specifically binds to a ROR1 polypeptide can be used to deplete a sample comprising a ROR1 polypeptide of said polypeptide or to purify said polypeptide.

To separate and purify a polypeptide or a protein of the present invention from the culture products, for example, after culture, microbial bodies or cells are collected by a known method, they are suspended in an appropriate buffer, the microbial bodies or the cells are disrupted by, for example, ultrasonic waves, lysozymes, and/or freeze-thawing, the resultant is then subjected to centrifugation or filtration, and then a crude extract of the protein can be obtained. The buffer may also contain a protein denaturation agent such as urea or guanidine hydrochloride or a surfactant such as Triton X-100(™). When the protein is secreted in a culture solution, microbial bodies or cells and a supernatant are separated by a known method after the completion of culture and then the supernatant is collected. The protein contained in the thus obtained culture supernatant or the extract can be purified by an appropriate combination of known separation and purification methods. The thus obtained polypeptide (protein) of the present invention can be converted into a salt by a known method or a method according thereto. Conversely, when the polypeptide (protein) of the present invention is obtained in the form of a salt, it can be converted into a free protein or peptide or another salt by a known method or a method according thereto. Moreover, an appropriate protein modification enzyme such as trypsin or chymotrypsin is caused to act on a protein produced by a recombinant before or after purification, so that modification can be arbitrarily added or a polypeptide can be partially removed. The presence of a polypeptide (protein) of the present invention or a salt thereof can be measured by various binding assays, enzyme immunoassays using specific antibodies, and the like.

Techniques well known in the art may be used for refolding to regenerate native or active conformations of the ROR1 polypeptide when the polypeptide has been denatured during isolation and or purification. In the context of the present invention, ROR1 polypeptide can be obtained from a biological sample from any source, such as and without limitation, a blood sample or tissue sample, e.g. a bladder, breast, colorectal, head and neck, kidney, liver, lung, ovarian, pancreatic, skin or thyroid tissue sample.

ROR1 polypeptide may be in the form of a “mature protein” or may be part of a larger protein such as a fusion protein. It is often advantageous to include an additional amino acid sequence which contains secretory or leader sequences, a pre-, pro- or prepro-protein sequence, or a sequence which aids in purification such as an affinity tag, for example, but without limitation, multiple histidine residues, a FLAG tag, HA tag or myc tag.

ROR1 may, for example, be fused with a heterologous fusion partner such as the surface protein, known as protein D from Haemophilus Influenza B, a non-structural protein from influenzae virus such as NS1, the S antigen from Hepatitis B or a protein known as LYTA such as the C terminal thereof.

An additional sequence that may provide stability during recombinant production may also be used. Such sequences may be optionally removed as required by incorporating a cleavable sequence as an additional sequence or part thereof. Thus, a ROR1 polypeptide may be fused to other moieties including other polypeptides or proteins (for example, glutathione S-transferase and protein A). Such a fusion protein can be cleaved using an appropriate protease, and then separated into each protein. Such additional sequences and affinity tags are well known in the art. In addition to the above, features known in the art, such as an enhancer, a splicing signal, a polyA addition signal, a selection marker, and an SV40 replication origin can be added to an expression vector, if desired.

Production of Affinity Reagents to ROR1

According to those in the art, there are three main types of immunoaffinity reagent—monoclonal antibodies, phage display antibodies and smaller antibody-derived molecules such as Affibodies, Domain Antibodies (dAbs), Nanobodies, UniBodies, DARPins, Anticalins, Duocalins, Avimers or Versabodies. In general in applications according to the present invention where the use of antibodies is stated, other affinity reagents (e.g. Affibodies, Domain Antibodies, Nanobodies, UniBodies, DARPins, Anticalins, Duocalins, Avimers or Versabodies) may be employed. Such substances may be said to be capable of immunospecific binding to ROR1. Where appropriate the term “affinity agent” shall be construed to embrace immunoaffinity reagents and other substances capable of specific binding to ROR1 including but not limited to ligands, lectins, streptavidins, antibody mimetics and synthetic binding agents.

Production of Antibodies to ROR1

According to the invention ROR1, a ROR1 analog, a ROR1-related protein or a fragment or derivative of any of the foregoing may be used as an immunogen to generate antibodies which immunospecifically bind such an immunogen. Such immunogens can be isolated by any convenient means, including the methods described above. The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites” (e.g. fragments, subsequences, complementarity determining regions (CDRs)) that retain capacity to bind antigen, including (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody”. Antibodies of the invention include, but are not limited to polyclonal, monoclonal, bispecific, humanized or chimeric antibodies, single chain antibodies, Fab fragments and F(ab′)₂ fragments, fragments produced by a Fab expression library, anti-idiotypic (anti-Id) antibodies, and epitope-binding fragments of any of the above. The immunoglobulin molecules of the invention can be of any class (e.g. IgG, IgE, IgM, IgD and IgA such as IgG) or subclass of immunoglobulin molecule.

The term “specifically binds” (or “immunospecifically binds”) is not intended to indicate that an antibody binds exclusively to its intended target. Rather, an antibody “specifically binds” if its affinity for its intended target is typically about 5-fold greater when compared to its affinity for a non-target molecule. Suitably there is no significant cross-reaction or cross-binding with undesired substances, especially naturally occurring proteins or tissues of a healthy person or animal Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In some embodiments, specific binding between an antibody or other binding agent and an antigen means a binding affinity of at least 10⁶ M⁻¹. Antibodies may, for example, bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation:

r/c=K(n−r):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g. U.S. Pat. No. 6,316,409). The affinity of a targeting agent for its target molecule is for example at least about 1×10⁻⁶ moles/liter, such as at least about 1×10⁻⁷ moles/liter, such as at least about 1×10⁻⁸ moles/liter, especially at least about 1×10⁻⁹ moles/liter, and particularly at least about 1×10⁻¹⁰ moles/liter. Antibody affinity measurement by Scatchard analysis is well known in the art. See, e.g. van Erp et al., J. Immunoassay 12: 425-43, 1991; Nelson and Griswold, Comput. Methods Programs Biomed. 27: 65-8, 1988.

In one embodiment, antibodies that recognize gene products of genes encoding ROR1 are publicly available. In another embodiment, methods known to those skilled in the art are used to produce antibodies that recognize ROR1, a ROR1 analog, a ROR1-related polypeptide, or a fragment or derivative of any of the foregoing. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In one embodiment of the invention, antibodies to a specific domain of ROR1 are produced. In a specific embodiment, hydrophilic fragments of ROR1 are used as immunogens for antibody production.

In the production of antibodies, screening for the desired antibody can be accomplished by techniques known in the art, e.g. ELISA (enzyme-linked immunosorbent assay). For example, to select antibodies which recognize a specific domain of ROR1, one may assay generated hybridomas for a product which binds to a ROR1 fragment containing such domain. For selection of an antibody that specifically binds a first ROR1 homolog but which does not specifically bind to (or binds less avidly to) a second ROR1 homolog, one can select on the basis of positive binding to the first ROR1 homolog and a lack of binding to (or reduced binding to) the second ROR1 homolog. Similarly, for selection of an antibody that specifically binds ROR1 but which does not specifically bind to (or binds less avidly to) a different isoform of the same protein (such as a different glycoform having the same core peptide as ROR1), one can select on the basis of positive binding to ROR1 and a lack of binding to (or reduced binding to) the different isoform (e.g. a different glycoform). Thus, the present invention provides an antibody (such as a monoclonal antibody) that binds with greater affinity (for example at least 2-fold, such as at least 5-fold, particularly at least 10-fold greater affinity) to ROR1 than to a different isoform or isoforms (e.g. glycoforms) of ROR1.

Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to ROR1, a fragment of ROR1, a ROR1-related polypeptide, or a fragment of a ROR1-related polypeptide. For example, one way is to purify polypeptides of interest or to synthesize the polypeptides of interest using, e.g. solid phase peptide synthesis methods well known in the art. See, e.g. Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be used to immunize by injection various host animals, including but not limited to rabbits, mice, rats, etc., to generate polyclonal or monoclonal antibodies. If ROR1 is purified by gel electrophoresis, ROR1 can be used for immunization with or without prior extraction from the polyacrylamide gel. Various adjuvants (i.e. immunostimulants) may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward ROR1, a fragment of ROR1, a ROR1-related polypeptide, or a fragment of a ROR1-related polypeptide, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g. human-mouse chimeras). A chimeric antibody is a molecule in which different portions are derived from different animal species, such as those having a human immunoglobulin constant region and a variable region derived from a murine mAb. (See, e.g. Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g. Queen, U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chain genes, but which can express human heavy and light chain genes. The transgenic mice are immunized in the normal fashion with a selected antigen, e.g. all or a portion of ROR1. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g. U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection”. In this approach a selected non-human monoclonal antibody, e.g. a mouse antibody, is used to guide the selection of a completely human antibody recognizing the same epitope. (Jespers et al. (1994) BioTechnology 12:899-903).

The antibodies of the present invention can also be generated by the use of phage display technology to produce and screen libraries of polypeptides for binding to a selected target. See, e.g. Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g. U.S. Pat. No. 6,057,098, which is hereby incorporated in its entirety, including all tables, figures, and claims. In particular, such phage can be utilized to display antigen binding domains expressed from a repertoire or combinatorial antibody library (e.g. human or murine). Phage expressing an antigen binding domain that binds the antigen of interest can be selected or identified with antigen, e.g. using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g. as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published 13 May 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al., Methods in Enzymology, 1986, 121:210.

The invention provides functionally active fragments, derivatives or analogs of the anti-ROR1 immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)₂ fragments and Fab fragments. Antibody fragments which recognize specific epitopes may be generated by known techniques. F(ab′)₂ fragments consist of the variable region, the light chain constant region and the CH1 domain of the heavy chain and are generated by pepsin digestion of the antibody molecule. Fab fragments are generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g. as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g. a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are modified, i.e., by the covalent attachment of any type of molecule as long as such covalent attachment does not impair immunospecific binding. For example, but not by way of limitation, the derivatives and analogs of the immunoglobulins include those that have been further modified, e.g. by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localization and activity of ROR1, e.g., for imaging this protein, measuring levels thereof in appropriate physiological samples, in diagnostic methods, etc.

Production of Affibodies to ROR1

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55.). The simple, robust structure of Affibody molecules in combination with their low molecular weight (6 kDa), make them suitable for a wide variety of applications, for instance, as detection reagents (Ronmark J, Hansson M, Nguyen T, et al, Construction and characterization of Affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

Labelled Affibodies may also be useful in imaging applications for determining abundance of Isoforms.

Production of Domain Antibodies to ROR1

References to antibodies herein embrace references to Domain Antibodies. Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, Domain Antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; US Serial No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

Production of Nanobodies to ROR1

Nanobodies are antibody-derived therapeutic proteins that contain the unique structural and functional properties of naturally-occurring heavy-chain antibodies. These heavy-chain antibodies contain a single variable domain (VHH) and two constant domains (C_{H}2 and C_{H}3). Importantly, the cloned and isolated VHH domain is a perfectly stable polypeptide harbouring the full antigen-binding capacity of the original heavy-chain antibody. Nanobodies have a high homology with the V_{H} domains of human antibodies and can be further humanised without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see e.g. WO 04/041867, which is herein incorporated by reference in its entirety) and are easy to manufacture. Other advantages of Nanobodies include recognising uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts e.g. E. coli (see e.g. U.S. Pat. No. 6,765,187, which is herein incorporated by reference in its entirety), moulds (for example Aspergillus or Trichoderma) and yeast (for example Saccharomyces, Kluyveromyces, Hansenula or Pichia) (see e.g. U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see e.g. WO 06/079372, which is herein incorporated by reference in its entirety) is a proprietary method for generating Nanobodies against a desired target, based on automated high-throughout selection of B-cells.

Production of UniBodies to ROR1

UniBodies are another antibody fragment technology; however this one is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, UniBodies may function to inhibit or silence, but not kill, the cells to which they are bound. Additionally, UniBody binding to cancer cells do not stimulate them to proliferate. Furthermore, because UniBodies are about half the size of traditional IgG4 antibodies, they may show better distribution over larger solid tumors with potentially advantageous efficacy. UniBodies are cleared from the body at a similar rate to whole IgG4 antibodies and are able to bind with a similar affinity for their antigens as whole antibodies. Further details of UniBodies may be obtained by reference to patent WO2007/059782, which is herein incorporated by reference in its entirety.

Production of DARPins to ROR1

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains.

DARPins can be produced in bacterial expression systems at very high yields and they belong to the most stable proteins known. Highly specific, high-affinity DARPins to a broad range of target proteins, including human receptors, cytokines, kinases, human proteases, viruses and membrane proteins, have been selected. DARPins having affinities in the single-digit nanomolar to picomolar range can be obtained.

DARPins have been used in a wide range of applications, including ELISA, sandwich ELISA, flow cytometric analysis (FACS), immunohistochemistry (IHC), chip applications, affinity purification or Western blotting. DARPins also proved to be highly active in the intracellular compartment for example as intracellular marker proteins fused to green fluorescent protein (GFP). DARPins were further used to inhibit viral entry with IC50 in the pM range. DARPins are not only ideal to block protein-protein interactions, but also to inhibit enzymes. Proteases, kinases and transporters have been successfully inhibited, most often an allosteric inhibition mode. Very fast and specific enrichments on the tumor and very favorable tumor to blood ratios make DARPins well suited for in vivo diagnostics or therapeutic approaches.

Additional information regarding DARPins and other DRP technologies can be found in US Patent Application Publication No. 2004/0132028, and International Patent Application Publication No. WO02/20565, both of which are hereby incorporated by reference in their entirety.

Production of Anticalins to ROR1

Anticalins are an additional antibody mimetic technology, however in this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids which is marginally larger than a single immunoglobulin domain.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein; they can be isolated and binding affinities in the nanomolar or higher range can be obtained.

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains.

Modulation of multiple targets through a single molecule is particularly advantageous in diseases known to involve more than a single causative factor. Moreover, bi- or multivalent binding formats such as Duocalins have significant potential in targeting cell surface molecules in disease, mediating agonistic effects on signal transduction pathways or inducing enhanced internalization effects via binding and clustering of cell surface receptors. Furthermore, the high intrinsic stability of Duocalins is comparable to monomeric Anticalins, offering flexible formulation and delivery potential for Duocalins.

Additional information regarding Anticalins can be found in U.S. Pat. No. 7,250,297 and International Patent Application Publication No. WO 99/16873, both of which are hereby incorporated by reference in their entirety.

Production of Avimers to ROR1

Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets.

Additional information regarding Avimers can be found in US Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Production of Versabodies to ROR1

Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

The inspiration for Versabodies comes from the natural injectable biopharmaceuticals produced by leeches, snakes, spiders, scorpions, snails, and anemones, which are known to exhibit unexpectedly low immunogenicity. Starting with selected natural protein families, by design and by screening the size, hydrophobicity, proteolytic antigen processing, and epitope density are minimized to levels far below the average for natural injectable proteins.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life.

Additional information regarding Versabodies can be found in US Patent Application Publication No. 2007/0191272 which is hereby incorporated by reference in its entirety.

Expression of Affinity Reagents

Expression of Antibodies

The antibodies of the invention can be produced by any method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g. as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g. an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, for example, by generating monoclonal antibodies. Alternatively, a clone encoding at least the Fab portion of the antibody may be obtained by screening Fab expression libraries (e.g. as described in Huse et al., 1989, Science 246:1275-1281) for clones of Fab fragments that bind the specific antigen or by screening antibody libraries (See, e.g. Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g. PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. USA 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g. humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2^{nd} Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus are an effective expression system for antibodies (Foecking et al., 1986, Gene 45:101; Cockett et al., 1990, BioTechnology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g. E. coli, B. subtilis) transformed with recombinant bacteriophage DNA, plasmid DNA or cosmid DNA expression vectors containing antibody coding sequences; yeast (e.g. Saccharomyces, Pichia) transformed with recombinant yeast expression vectors containing antibody coding sequences; insect cell systems infected with recombinant virus expression vectors (e.g. baculovirus) containing the antibody coding sequences; plant cell systems infected with recombinant virus expression vectors (e.g. cauliflower mosaic virus, CaMV; tobacco mosaic virus, TMV) or transformed with recombinant plasmid expression vectors (e.g. Ti plasmid) containing antibody coding sequences; or mammalian cell systems (e.g. COS, CHO, BHK, 293, 3T3 cells) harboring recombinant expression constructs containing promoters derived from the genome of mammalian cells (e.g. metallothionein promoter) or from mammalian viruses (e.g. the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. The pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g. an adenovirus expression system) may be utilized.

As discussed above, a host cell strain may be chosen which modulates the expression of the inserted sequences, or modifies and processes the gene product in the specific fashion desired. Such modifications (e.g. glycosylation) and processing (e.g. cleavage) of protein products may be important for the function of the protein.

For long-term, high-yield production of recombinant antibodies, stable expression is preferred. For example, cell lines that stably express an antibody of interest can be produced by transfecting the cells with an expression vector comprising the nucleotide sequence of the antibody and the nucleotide sequence of a selectable (e.g. neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g. ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni²⁺ nitriloacetic acid-agarose columns and histidine-tagged proteins are selectively eluted with imidazole-containing buffers.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) is present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g. in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

For therapeutic applications, antibodies (particularly monoclonal antibodies) may suitably be human or humanized animal (e.g. mouse) antibodies Animal antibodies may be raised in animals using the human protein (e.g. ROR1) as immunogen. Humanisation typically involves grafting CDRs identified thereby into human framework regions. Normally some subsequent retromutation to optimize the conformation of chains is required. Such processes are known to persons skilled in the art.

Expression of Affibodies

The construction of affibodies has been described elsewhere (Ronnmark J, Gronlund H, Uhlen, M., Nygren P. A, Human immunoglobulin A (IgA)-specific ligands from combinatorial engineering of protein A, 2002, Eur. J. Biochem. 269, 2647-2655.), including the construction of Affibody phage display libraries (Nord, K., Nilsson, J., Nilsson, B., Uhlen, M. & Nygren, P. A, A combinatorial library of an a-helical bacterial receptor domain, 1995, Protein Eng. 8, 601-608. Nord, K., Gunneriusson, E., Ringdahl, J., Stahl, S., Uhlen, M. & Nygren, P. A, Binding proteins selected from combinatorial libraries of an a-helical bacterial receptor domain, 1997, Nat. Biotechno1.15, 772-777.)

The biosensor analyses to investigate the optimal Affibody variants using biosensor binding studies has also been described elsewhere (Ronnmark J, Gronlund H, Uhlen, M., Nygren P. A, Human immunoglobulin A (IgA)-specific ligands from combinatorial engineering of protein A, 2002, Eur. J. Biochem. 269, 2647-2655.).

Affinity Reagent Modifications

In a preferred embodiment, anti-ROR1 affinity reagents such as antibodies or fragments thereof are conjugated to a diagnostic moiety (such as a detectable label) or a therapeutic moiety. The antibodies can be used for diagnosis or to determine the efficacy of a given treatment regimen. Detection can be facilitated by coupling the antibody to a detectable substance (label). Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹ I, ¹¹¹In and ⁹⁹Tc. ⁶⁸Ga may also be employed.

As indicated above affinity reagents, such as antibodies for use in the invention, may be conjugated to a therapeutic moiety, such as a cytotoxin, a drug (e.g., an immunosuppressant) or a radiotoxin. Such conjugates are referred to herein as “immunoconjugates” Immunoconjugates that include one or more cytotoxins are referred to as “immunotoxins”. A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other preferred examples of therapeutic cytotoxins that can be conjugated to an antibody of the invention include duocarmycins, calicheamicins, maytansines and auristatins, and derivatives thereof. An example of a calicheamicin antibody conjugate is commercially available (Mylotarg®; American Home Products).

Cytotoxins can be conjugated to antibodies of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

Examples of cytotoxins are described, for example, in U.S. Pat. Nos. 6,989,452, 7,087,600, and 7,129,261, and in PCT Application Nos. PCT/US2002/17210, PCT/US2005/017804, PCT/US2006/37793, PCT/US2006/060050, PCT/US2006/060711, WO2006/110476, and in U.S. Patent Application No. 60/891,028, all of which are incorporated herein by reference in their entirety. For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Affinity reagents can also be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine131, indium111, yttrium90 and lutetium177. Method for preparing radioimmunoconjugates are established in the art. Examples of radioimmunoconjugates are commercially available, including Zevalin® (IDEC Pharmaceuticals) and Bexxar® (Corixa Pharmaceuticals), and similar methods can be used to prepare radioimmunoconjugates using the antibodies of the invention.

The conjugates can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Senter P. D. (2009) Curr. Opin. Chem. Biol. 13(3):235-244; Kovtun et al. (2010) Cancer Res. 70(6):2528-2537.

Techniques for conjugating such therapeutic moieties to antibodies are well known, see, e.g., Amon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985), and Thorpe et al., Immunol. Rev., 62:119-58 (1982).

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

The invention also provides for fully human, or humanised antibodies that induce antibody-directed cell-mediated cytotoxicity (ADCC). A fully human antibody is one in which the protein sequences are encoded by naturally occurring human immunoglobulin sequences, either from isolated antibody-producing human B-lymphocytes, or from transgenic murine B-lymphocytes of mice in which the murine immunoglobulin coding chromosomal regions have been replaced by orthologous human sequences. Transgenic antibodies of the latter type include, but are not restricted to, HuMab (Medarex, Inc, CA) and XenoMouse (Abgenix Inc., CA). A humanised antibody is one in which the constant region of a non-human antibody molecule of appropriate antigen specificity, is replaced by the constant region of a human antibody, preferably of the IgG subtype, with appropriate effector functions (Morrison et al., 1984, Proc. Natl. Acad. Sci. 81:851-855; Neuberger et al., 1984, Nature 312:604-608; Takeda et al., 1985, Nature 314:452-454). Appropriate effector functions include ADCC, which is a natural process by which fully-human antibodies or humanized antibodies, when bound to targets on the surface of cancer cells, switch on the cell killing properties of lymphocytes that are part of the normal immune system. These active lymphocytes, called Natural Killer (NK) cells, use a cytotoxic process to destroy living cells to which the antibodies are bound. ADCC activity may be detected and quantified by measuring release of Europium (Eu³⁺) from Eu³⁺ labelled, living cells in the presence of an antigen-specific antibody and peripheral blood mononuclear cells extracted from an immunocompetent, living human subject. The ADCC process is described in detail in Janeway Jr. C. A. et al., Immunobiology, 5th ed., 2001, Garland Publishing, ISBN 0-8153-3642-X; Pier G. B. et al., Immunology, Infection, and Immunity, 2004, p246-5; Albanell J. et al., Advances in Experimental Medicine and Biology, 2003, 532:p 2153-68 and Weng, W.-K. et al., Journal of Clinical Oncology, 2003, 21:p 3940-3947. Suitable methods for the detection and quantification of ADCC can be found in Blomberg et al., Journal of Immunological Methods. 1986, 86:p 225-9; Blomberg et al., Journal of Immunological Methods. 1986, 21; 92:p 117-23 and Patel & Boyd, Journal of Immunological Methods. 1995, 184:p 29-38.

ADCC typically involves activation of NK cells and is dependent on the recognition of antibody-coated cells by Fc receptors on the surface of the NK cell. The Fc receptors recognize the Fc (crystalline) portion of antibodies such as IgG, bound specifically to the surface of a target cell. The Fc receptor that triggers activation of the NK cell is called CD 16 or FcγRIIIa. Once the FcγRIIIa receptor is bound to the IgG Fc, the NK cell releases cytokines such as IFN-γ, and cytotoxic granules containing perform and granzymes that enter the target cell and promote cell death by triggering apoptosis.

The induction of antibody-dependent cellular cytotoxicity (ADCC) by an antibody can be enhanced by modifications that alter interactions between the antibody constant region (Fc) and various receptors that are present on the surface of cells of the immune system. Such modifications include the reduction or absence of alpha1,6-linked fucose moieties in the complex oligosaccharide chains that are normally added to the Fc of antibodies during natural or recombinant synthesis in mammalian cells. In a preferred embodiment, non-fucosylated anti-ROR1 affinity reagents such as antibodies or fragments thereof are produced for the purpose of enhancing their ability to induce the ADCC response.

Techniques for reducing or ablating alpha 1,6-linked fucose moieties in the oligosaccharide chains of the Fc are well established. In one example, the recombinant antibody is synthesized in a cell line that is impaired in its ability to add fucose in an alpha 1,6 linkage to the innermost N-acetylglucosamine of the N-linked biantennary complex-type Fc oligosaccharides. Such cell lines include, but are not limited to, the rat hybridoma YB2/0, which expresses a reduced level of the alpha 1,6-fucosyltransferase gene, FUT8. Preferably, the antibody is synthesized in a cell line that is incapable of adding alpha 1,6-linked fucosyl moieties to complex oligosaccharide chains, due to the deletion of both copies of the FUT8 gene. Such cell lines include, but are not limited to, FUT8−/− CHO/DG44 cell lines. Techniques for synthesizing partially fucosylated, or non-fucosylated antibodies and affinity reagents are described in Shinkawa et al., J. Biol. Chem. 278:3466-34735 (2003); Yamane-Ohnuki et al., Biotechnology and Bioengineering 87: 614-22 (2004) and in WO00/61739 A1, WO02/31140 A1 and WO03/085107 A1. In a second example, the fucosylation of a recombinant antibody is reduced or abolished by synthesis in a cell line that has been genetically engineered to overexpress a glycoprotein-modifying glycosyl transferase at a level that maximizes the production of complex N-linked oligosaccharides carrying bisecting N-acetylglucosamine. For example, the antibody is synthesized in a Chinese Hamster Ovary cell line expressing the enzyme N-acetyl glucosamine transferase III (GnT III). Cell lines stably transfected with suitable glycoprotein-modifying glycosyl transferases, and methods of synthesizing antibodies using these cells are described in WO99/54342.

A non-fucosylated antibody or affinity reagent can be used as a therapeutic that is administered alone or in combination with cytotoxic factor(s) and/or cytokine(s).

In a further modification, the amino acid sequences of the antibody Fc are altered in a way that enhances ADCC activation, without affecting ligand affinity. Examples of such modifications are described in Lazar et al., Proceedings of the National Academy of Sciences 2006, 103: p 4005-4010; WO03/074679 and WO2007/039818. In these examples, substitution of amino acids in the antibody Fc, such as aspartate for serine at position 239, and isoleucine for glutamate at position 332, altered the binding affinity of an antibody for Fc receptors, leading to an increase in ADCC activation.

An antibody reagent with enhanced ADCC activation due to amino acid substitutions can be used as a therapeutic that is administered alone or in combination with cytotoxic factor(s) and/or cytokine(s).

Diagnosis of Cancer Including Bladder Cancer, Breast Cancer, Colorectal Cancer, Head and Neck Cancer, Kidney Cancer, Liver Cancer, Lung Cancer, Ovarian Cancer, Pancreatic Cancer, Skin Cancer or Thyroid Cancer

In accordance with the present invention, test samples of e.g. bladder, breast, colorectal, head and neck, kidney, liver, lung, ovarian, pancreatic, skin or thyroid tissue, serum, plasma or urine obtained from a subject suspected of having or known to have bladder cancer, breast cancer, colorectal cancer, head and neck cancer, kidney cancer, liver cancer, lung cancer, ovarian cancer, pancreatic cancer, skin cancer or thyroid cancer can be used for diagnosis or monitoring. In one embodiment, a change in the abundance of ROR1 in a test sample relative to a control sample (from a subject or subjects free from bladder cancer, breast cancer, colorectal cancer, head and neck cancer, kidney cancer, liver cancer, lung cancer, ovarian cancer, pancreatic cancer, skin cancer and thyroid cancer) or a previously determined reference range indicates the presence of bladder cancer, breast cancer, colorectal cancer, head and neck cancer, kidney cancer, liver cancer, lung cancer, ovarian cancer, pancreatic cancer, skin cancer or thyroid cancer. In another embodiment, the relative abundance of ROR1 in a test sample compared to a control sample or a previously determined reference range indicates a subtype of bladder cancer, breast cancer, colorectal cancer, head and neck cancer, kidney cancer, liver cancer, lung cancer, ovarian cancer, pancreatic cancer, skin cancer or thyroid cancer (e.g. squamous cell bladder carcinoma, inflammatory breast cancer, familial or sporadic colorectal cancer, nasopharyngeal cancer, fibrolamellar hepatocellular carcinoma, squamous cell lung carcinoma, malignant papillary serous adenocarcinoma, endocrine tumours of the pancreas, squamous cell skin carcinoma or anaplastic thyroid carcinoma). In yet another embodiment, the relative abundance of ROR1 in a test sample relative to a control sample or a previously determined reference range indicates the degree or severity of bladder cancer, breast cancer, colorectal cancer, head and neck cancer, kidney cancer, liver cancer, lung cancer, ovarian cancer, pancreatic cancer, skin cancer or thyroid cancer (e.g. the likelihood for metastasis). In any of the aforesaid methods, detection of ROR1 may optionally be combined with detection of one or more of additional biomarkers for bladder cancer, breast cancer, colorectal cancer, head and neck cancer, kidney cancer, liver cancer, lung cancer, ovarian cancer, pancreatic cancer, skin cancer or thyroid cancer. Any suitable method in the art can be employed to measure the level of ROR1, including but not limited to the Preferred Technologies described herein, kinase assays, immunoassays to detect and/or visualize the ROR1 (e.g. Western blot, immunoprecipitation followed by sodium dodecyl sulfate polyacrylamide gel electrophoresis, immunocytochemistry, etc.). In a further embodiment, a change in the abundance of mRNA encoding ROR1 in a test sample relative to a control sample or a previously determined reference range indicates the presence of bladder cancer, breast cancer, colorectal cancer, head and neck cancer, kidney cancer, liver cancer, lung cancer, ovarian cancer, pancreatic cancer, skin cancer or thyroid cancer. Any suitable hybridization assay can be used to detect ROR1 expression by detecting and/or visualizing mRNA encoding the ROR1 (e.g. Northern assays, dot blots, in situ hybridization, etc.).

In another embodiment of the invention, labeled antibodies (or other affinity reagents), derivatives and analogs thereof, which specifically bind to ROR1 can be used for diagnostic purposes to detect, diagnose, or monitor bladder cancer, breast cancer, colorectal cancer, head and neck cancer, kidney cancer, liver cancer, lung cancer, ovarian cancer, pancreatic cancer, skin cancer or thyroid cancer. Preferably, bladder cancer, breast cancer, colorectal cancer, head and neck cancer, kidney cancer, liver cancer, lung cancer, ovarian cancer, pancreatic cancer, skin cancer or thyroid cancer is detected in an animal, more preferably in a mammal and most preferably in a human.

Screening Assays

The invention provides methods for identifying agents (e.g. candidate compounds or test compounds) that bind to ROR1 or have a stimulatory or inhibitory effect on the expression or activity of ROR1. The invention also provides methods of identifying agents, candidate compounds or test compounds that bind to a ROR1-related polypeptide or a ROR1 fusion protein or have a stimulatory or inhibitory effect on the expression or activity of a ROR1-related polypeptide or a ROR1 fusion protein. Examples of agents, candidate compounds or test compounds include, but are not limited to, nucleic acids (e.g. DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented, e.g. presented in solution (e.g. Houghten, 1992, BioTechniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment, agents that interact with (i.e. bind to) ROR1, a ROR1 fragment (e.g. a functionally active fragment), a ROR1-related polypeptide, a fragment of a ROR1-related polypeptide, or a ROR1 fusion protein are identified in a cell-based assay system. In accordance with this embodiment, cells expressing ROR1, a fragment of a ROR1, a ROR1-related polypeptide, a fragment of the ROR1-related polypeptide, or a ROR1 fusion protein are contacted with a candidate compound or a control compound and the ability of the candidate compound to interact with the ROR1 is determined. If desired, this assay may be used to screen a plurality (e.g. a library) of candidate compounds. The cell, for example, can be of prokaryotic origin (e.g. E. coli) or eukaryotic origin (e.g. yeast or mammalian). Further, the cells can express ROR1, a fragment of ROR1, a ROR1-related polypeptide, a fragment of the ROR1-related polypeptide, or a ROR1 fusion protein endogenously or be genetically engineered to express ROR1, a fragment of ROR1, a ROR1-related polypeptide, a fragment of the ROR1-related polypeptide, or a ROR1 fusion protein. In certain instances, ROR1, a fragment of ROR1, a ROR1-related polypeptide, a fragment of the ROR1-related polypeptide, or a ROR1 fusion protein or the candidate compound is labeled, for example with a radioactive label (such as ³²P, ³⁵S, and ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between ROR1 and a candidate compound. The ability of the candidate compound to interact directly or indirectly with ROR1, a fragment of a ROR1, a ROR1-related polypeptide, a fragment of a ROR1-related polypeptide, or a ROR1 fusion protein can be determined by methods known to those of skill in the art. For example, the interaction between a candidate compound and ROR1, a ROR1-related polypeptide, a fragment of a ROR1-related polypeptide, or a ROR1 fusion protein can be determined by flow cytometry, a scintillation assay, immunoprecipitation or western blot analysis.

In another embodiment, agents that interact with (i.e. bind to) ROR1, a ROR1 fragment (e.g. a functionally active fragment), a ROR1-related polypeptide, a fragment of a ROR1-related polypeptide, or a ROR1 fusion protein are identified in a cell-free assay system. In accordance with this embodiment, native or recombinant ROR1 or a fragment thereof, or a native or recombinant ROR1-related polypeptide or fragment thereof, or a ROR1-fusion protein or fragment thereof, is contacted with a candidate compound or a control compound and the ability of the candidate compound to interact with ROR1 or ROR1-related polypeptide, or ROR1 fusion protein is determined. If desired, this assay may be used to screen a plurality (e.g. a library) of candidate compounds. Preferably, ROR1, a ROR1 fragment, a ROR1-related polypeptide, a fragment of a ROR1-related polypeptide, or a ROR1-fusion protein is first immobilized, by, for example, contacting ROR1, a ROR1 fragment, a ROR1-related polypeptide, a fragment of a ROR1-related polypeptide, or a ROR1 fusion protein with an immobilized antibody (or other affinity reagent) which specifically recognizes and binds it, or by contacting a purified preparation of ROR1, a ROR1 fragment, a ROR1-related polypeptide, fragment of a ROR1-related polypeptide, or a ROR1 fusion protein with a surface designed to bind proteins. ROR1, a ROR1 fragment, a ROR1-related polypeptide, a fragment of a ROR1-related polypeptide, or a ROR1 fusion protein may be partially or completely purified (e.g. partially or completely free of other polypeptides) or part of a cell lysate. Further, ROR1, a ROR1 fragment, a ROR1-related polypeptide, or a fragment of a ROR1-related polypeptide may be a fusion protein comprising ROR1 or a biologically active portion thereof, or ROR1-related polypeptide and a domain such as glutathionine-5-transferase. Alternatively, ROR1, a ROR1 fragment, a ROR1-related polypeptide, a fragment of a ROR1-related polypeptide or a ROR1 fusion protein can be biotinylated using techniques well known to those of skill in the art (e.g. biotinylation kit, Pierce Chemicals; Rockford, Ill.). The ability of the candidate compound to interact with ROR1, a ROR1 fragment, a ROR1-related polypeptide, a fragment of a ROR1-related polypeptide, or a ROR1 fusion protein can be determined by methods known to those of skill in the art.

In another embodiment, a cell-based assay system is used to identify agents that bind to or modulate the activity of a protein, such as an enzyme, or a biologically active portion thereof, which is responsible for the production or degradation of ROR1 or is responsible for the post-translational modification of ROR1. In a primary screen, a plurality (e.g. a library) of compounds are contacted with cells that naturally or recombinantly express: (i) ROR1, an isoform of ROR1, a ROR1 homolog, a ROR1-related polypeptide, a ROR1 fusion protein, or a biologically active fragment of any of the foregoing; and (ii) a protein that is responsible for processing of ROR1, a ROR1 isoform, a ROR1 homolog, a ROR1-related polypeptide, a ROR1 fusion protein, or a fragment in order to identify compounds that modulate the production, degradation, or post-translational modification of ROR1, a ROR1 isoform, a ROR1 homolog, a ROR1-related polypeptide, a ROR1 fusion protein or fragment. If desired, compounds identified in the primary screen can then be assayed in a secondary screen against cells naturally or recombinantly expressing ROR1. The ability of the candidate compound to modulate the production, degradation or post-translational modification of ROR1, isoform, homolog, ROR1-related polypeptide, or ROR1 fusion protein can be determined by methods known to those of skill in the art, including without limitation, flow cytometry, a scintillation assay, immunoprecipitation and western blot analysis.

In another embodiment, agents that competitively interact with (i.e. bind to) ROR1, a ROR1 fragment, a ROR1-related polypeptide, a fragment of a ROR1-related polypeptide, or a ROR1 fusion protein are identified in a competitive binding assay. In accordance with this embodiment, cells expressing ROR1, a ROR1 fragment, a ROR1-related polypeptide, a fragment of a ROR1-related polypeptide, or a ROR1 fusion protein are contacted with a candidate compound and a compound known to interact with ROR1, a ROR1 fragment, a ROR1-related polypeptide, a fragment of a ROR1-related polypeptide or a ROR1 fusion protein; the ability of the candidate compound to preferentially interact with ROR1, a ROR1 fragment, a ROR1-related polypeptide, a fragment of a ROR1-related polypeptide, or a ROR1 fusion protein is then determined Alternatively, agents that preferentially interact with (i.e. bind to) ROR1, a ROR1 fragment, a ROR1-related polypeptide or fragment of a ROR1-related polypeptide are identified in a cell-free assay system by contacting ROR1, a ROR1 fragment, a ROR1-related polypeptide, a fragment of a ROR1-related polypeptide, or a ROR1 fusion protein with a candidate compound and a compound known to interact with ROR1, a ROR1-related polypeptide or a ROR1 fusion protein. As stated above, the ability of the candidate compound to interact with ROR1, a ROR1 fragment, a ROR1-related polypeptide, a fragment of a ROR1-related polypeptide, or a ROR1 fusion protein can be determined by methods known to those of skill in the art. These assays, whether cell-based or cell-free, can be used to screen a plurality (e.g. a library) of candidate compounds.

In another embodiment, agents that modulate (i.e. upregulate or downregulate) the expression or activity of ROR1 or a ROR1-related polypeptide are identified by contacting cells (e.g. cells of prokaryotic origin or eukaryotic origin) expressing ROR1 or a ROR1-related polypeptide with a candidate compound or a control compound (e.g. phosphate buffered saline (PBS)) and determining the expression of ROR1, ROR1-related polypeptide, or ROR1 fusion protein, mRNA encoding ROR1, or mRNA encoding the ROR1-related polypeptide. The level of expression of ROR1, ROR1-related polypeptide, mRNA encoding ROR1, or mRNA encoding the ROR1-related polypeptide in the presence of the candidate compound is compared to the level of expression of ROR1, ROR1-related polypeptide, mRNA encoding ROR1, or mRNA encoding the ROR1-related polypeptide in the absence of the candidate compound (e.g. in the presence of a control compound). The candidate compound can then be identified as a modulator of the expression of ROR1, or the ROR1-related polypeptide based on this comparison. For example, when expression of ROR1 or mRNA is significantly greater in the presence of the candidate compound than in its absence, the candidate compound is identified as a stimulator of expression of ROR1 or mRNA. Alternatively, when expression of ROR1 or mRNA is significantly less in the presence of the candidate compound than in its absence, the candidate compound is identified as an inhibitor of the expression of ROR1 or mRNA. The level of expression of ROR1 or the mRNA that encodes it can be determined by methods known to those of skill in the art. For example, mRNA expression can be assessed by Northern blot analysis or RT-PCR, and protein levels can be assessed by western blot analysis.

In another embodiment, agents that modulate the activity of ROR1 or a ROR1-related polypeptide are identified by contacting a preparation containing ROR1 or ROR1-related polypeptide or cells (e.g. prokaryotic or eukaryotic cells) expressing ROR1 or ROR1-related polypeptide with a test compound or a control compound and determining the ability of the test compound to modulate (e.g. stimulate or inhibit) the activity of ROR1 or ROR1-related polypeptide. The activity of ROR1 or a ROR1-related polypeptide can be assessed by detecting induction of a cellular signal transduction pathway of ROR1 or ROR1-related polypeptide (e.g. intracellular Ca²⁺, diacylglycerol, IP3, etc.), detecting catalytic or enzymatic activity of the target on a suitable substrate, detecting the induction of a reporter gene (e.g. a regulatory element that is responsive to ROR1 or a ROR1-related polypeptide and is operably linked to a nucleic acid encoding a detectable marker, e.g. luciferase), or detecting a cellular response, for example, cellular differentiation, or cell proliferation. Based on the present description, techniques known to those of skill in the art can be used for measuring these activities (see, e.g. U.S. Pat. No. 5,401,639, which is incorporated herein by reference). The candidate compound can then be identified as a modulator of the activity of ROR1 or a ROR1-related polypeptide by comparing the effects of the candidate compound to the control compound. Suitable control compounds include phosphate buffered saline (PBS) and normal saline (NS).

In another embodiment, agents that modulate (i.e. upregulate or downregulate) the expression, activity or both the expression and activity of ROR1 or a ROR1-related polypeptide are identified in an animal model. Examples of suitable animals include, but are not limited to, mice, rats, rabbits, monkeys, guinea pigs, dogs and cats. Preferably, the animal used represent a model of bladder cancer, breast cancer, colorectal cancer, head and neck cancer, liver cancer, lung cancer, ovarian cancer, pancreatic cancer, skin cancer or thyroid cancer (e.g. xenografts of bladder cancer cell lines such as UCRU-BL-12, UCRU-BL-13 and UCRU-BL-14, Russell et al. Cancer Res. 1986 April; 46(4 Pt 2):2035-40; xenografts of breast cancer cell lines such as MCF-7 (Ozzello L, Sordat M., Eur J Cancer. 1980; 16:553-559) and MCF10AT (Miller et al., J Natl Cancer Inst. 1993; 85:1725-1732) in nude or SCID mice; xenografts of human colorectal cancer cell lines such as MDA-MB-345 in oestrogen-deprived SCID mice, Eccles et al. 1994 Cell Biophysics 24/25, 279; xenografts of head and neck cancer cell lines such as FaDu and HNX-OE; xenografts of liver cancer cell lines such as MHCC97 in nude mice, Tian et al., Br J. Cancer 1999 November; 81(5):814-21; xenografts of non small cell lung cancer cell lines such as A549 and H460 and xenografts of small cell lung cancer cell lines such as NCI-H345; xenografts of ovarian cancer cell lines such as IGROV1 in nude mice, Benard et al, Cancer Res. 1985 October; 45(10):4970-9; xenografts of pancreatic cancer cell lines such as MIA PaCa-2 in nude mice, Marincola et al., J Surg Res 1989 December; 47(6):520-9; xenografts of skin cancer cell lines such as MV3 in nude mice, van Muijen et al., Int J Cancer 1991 Apr. 22; 48(1):85-91 or xenografts of thyroid cancer cell lines such as ARO, Viaggi et al., Thyroid 2003 June; 13(6):529-36). These can be utilized to test compounds that modulate ROR1 levels, since the pathology exhibited in these models is similar to that of e.g. bladder cancer, breast cancer, colorectal cancer, head and neck cancer, kidney cancer, liver cancer, lung cancer, ovarian cancer, pancreatic cancer, skin cancer or thyroid cancer. In accordance with this embodiment, the test compound or a control compound is administered (e.g. orally, rectally or parenterally such as intraperitoneally or intravenously) to a suitable animal and the effect on the expression, activity or both expression and activity of ROR1 or ROR1-related polypeptide is determined Changes in the expression of ROR1 or a ROR1-related polypeptide can be assessed by the methods outlined above.

In yet another embodiment, ROR1 or a ROR1-related polypeptide is used as a “bait protein” in a two-hybrid assay or three hybrid assay to identify other proteins that bind to or interact with ROR1 or a ROR1-related polypeptide (see, e.g. U.S. Pat. No. 5,283,317; Zervos et al. (1993) Cell 72:223-232; Madura et al. (1993) J. Biol. Chem. 268:12046-12054; Bartel et al. (1993) BioTechniques 14:920-924; Iwabuchi et al. (1993) Oncogene 8:1693-1696; and PCT Publication No. WO 94/10300). As those skilled in the art will appreciate, such binding proteins are also likely to be involved in the propagation of signals by ROR1 as, for example, upstream or downstream elements of a signaling pathway involving ROR1.

This invention further provides novel agents identified by the above-described screening assays and uses thereof for treatments as described herein. In addition, the invention also provides the use of an agent which interacts with, or modulates the activity of, ROR1 in the manufacture of a medicament for the treatment of bladder cancer, breast cancer, colorectal cancer, head and neck cancer, liver cancer, lung cancer, ovarian cancer, pancreatic cancer, skin cancer or thyroid cancer.

Therapeutic Use of ROR1

The invention provides for treatment or prevention of various diseases and disorders by administration of a therapeutic compound. Such compounds include but are not limited to: ROR1, ROR1 analogs, ROR1-related polypeptides and derivatives (including fragments) thereof; antibodies (or other affinity reagents) to the foregoing; nucleic acids encoding ROR1, ROR1 analogs, ROR1-related polypeptides and fragments thereof; antisense nucleic acids to a gene encoding ROR1 or a ROR1-related polypeptide; and modulator (e.g. agonists and antagonists) of a gene encoding ROR1 or a ROR1-related polypeptide. An important feature of the present invention is the identification of genes encoding the 1 on-variant of ROR1 involved in cancers such as bladder cancer, breast cancer, colorectal cancer, head and neck cancer, liver cancer, lung cancer, ovarian cancer, pancreatic cancer, skin cancer or thyroid cancer. Bladder cancer, breast cancer, colorectal cancer, head and neck cancer, liver cancer, lung cancer, ovarian cancer, pancreatic cancer, skin cancer or thyroid cancer, for example, can be treated (e.g. to ameliorate symptoms or to retard onset or progression) or prevented by administration of a therapeutic compound that reduces function or expression of ROR1 in the serum or tissue of subjects having bladder cancer, breast cancer, colorectal cancer, head and neck cancer, liver cancer, lung cancer, ovarian cancer, pancreatic cancer, skin cancer or thyroid cancer.

In one embodiment, one or more antibodies (or other affinity reagents) each specifically binding to ROR1 are administered alone or in combination with one or more additional therapeutic compounds or treatments.

A biological product such as an antibody (or other affinity reagent) is allogeneic to the subject to which it is administered. In one embodiment, a human ROR1 or a human ROR1-related polypeptide, a nucleotide sequence encoding a human ROR1 or a human ROR1-related polypeptide, or an antibody (or other affinity reagent) to a human ROR1 or a human ROR1-related polypeptide, is administered to a human subject for therapy (e.g. to ameliorate symptoms or to retard onset or progression) or prophylaxis.

Without being limited by theory, it is conceived that the therapeutic activity of antibodies (or other affinity reagents) which specifically bind to ROR1 may be achieved through the phenomenon of Antibody—Dependent Cell-mediated Cytotoxicity (ADCC) (see e.g. Janeway Jr. C. A. et al., Immunobiology, 5th ed., 2001, Garland Publishing, ISBN 0-8153-3642-X; Pier G. B. et al., Immunology, Infection, and Immunity, 2004, p 246-5; Albanell J. et al., Advances in Experimental Medicine and Biology, 2003, 532:p 2153-68 and Weng, W.-K. et al., Journal of Clinical Oncology, 2003, 21:p 3940-3947).

Treatment and Prevention of Bladder Cancer, Breast Cancer, Colorectal Cancer, Head and Neck Cancer, Kidney Cancer, Liver Cancer, Lung Cancer, Ovarian Cancer, Pancreatic Cancer, Skin Cancer or Thyroid Cancer

Bladder cancer, breast cancer, colorectal cancer, head and neck cancer, kidney cancer, liver cancer, lung cancer, ovarian cancer, pancreatic cancer, skin cancer or thyroid cancer, for example, is treated or prevented by administration to a subject suspected of having or known to have bladder cancer, breast cancer, colorectal cancer, head and neck cancer, kidney cancer, liver cancer, lung cancer, ovarian cancer, pancreatic cancer, skin cancer or thyroid cancer or to be at risk of developing bladder cancer, breast cancer, colorectal cancer, head and neck cancer, kidney cancer, liver cancer, lung cancer, ovarian cancer, pancreatic cancer, skin cancer or thyroid cancer of a compound that modulates (i.e. increases or decreases) the level or activity (i.e. function) of ROR1 that is differentially present in the serum or tissue of subjects having bladder cancer, breast cancer, colorectal cancer, head and neck cancer, kidney cancer, liver cancer, lung cancer, ovarian cancer, pancreatic cancer, skin cancer or thyroid cancer compared with serum or tissue of subjects free from bladder cancer, breast cancer, colorectal cancer, head and neck cancer, kidney cancer, liver cancer, lung cancer, ovarian cancer, pancreatic cancer, skin cancer and thyroid cancer. In one embodiment, bladder cancer, breast cancer, colorectal cancer, head and neck cancer, kidney cancer, liver cancer, lung cancer, ovarian cancer, pancreatic cancer, skin cancer or thyroid cancer is treated or prevented by administering to a subject suspected of having or known to have bladder cancer, breast cancer, colorectal cancer, head and neck cancer, kidney cancer, liver cancer, lung cancer, ovarian cancer, pancreatic cancer, skin cancer or thyroid cancer or to be at risk of developing bladder cancer, breast cancer, colorectal cancer, head and neck cancer, kidney cancer, liver cancer, lung cancer, ovarian cancer, pancreatic cancer, skin cancer or thyroid cancer a compound that upregulates (i.e. increases) the level or activity (i.e. function) of ROR1 that are decreased in the serum or tissue of subjects having bladder cancer, breast cancer, colorectal cancer, head and neck cancer, kidney cancer, liver cancer, lung cancer, ovarian cancer, pancreatic cancer, skin cancer or thyroid cancer. Examples of such a compound include, but are not limited to, ROR1 antisense oligonucleotides, ribozymes, antibodies (or other affinity reagents) directed against ROR1, and compounds that inhibit the enzymatic activity of ROR1. Other useful compounds e.g. ROR1 antagonists and small molecule ROR1 antagonists, can be identified using in vitro assays.

Cancer e.g. bladder cancer, breast cancer, colorectal cancer, head and neck cancer, kidney cancer, liver cancer, lung cancer, ovarian cancer, pancreatic cancer, skin cancer or thyroid cancer is also treated or prevented by administration to a subject suspected of having or known to have such cancer, or to be at risk of developing such cancer, of a compound that downregulates the level or activity (i.e. function) of ROR1 that are increased in the serum or tissue of subjects having such cancer. Examples of such a compound include but are not limited to: ROR1, ROR1 fragments and ROR1-related polypeptides; nucleic acids encoding ROR1, a ROR1 fragment and a ROR1-related polypeptide (e.g. for use in gene therapy); and, for those ROR1 or ROR1-related polypeptides with enzymatic activity, compounds or molecules known to modulate that enzymatic activity. Other compounds that can be used, e.g. ROR1 agonists, can be identified using in in vitro assays.

In another embodiment, therapy or prophylaxis is tailored to the needs of an individual subject. Thus, in specific embodiments, compounds that promote the level or function of ROR1 are therapeutically or prophylactically administered to a subject suspected of having or known to have cancer e.g. bladder cancer, breast cancer, colorectal cancer, head and neck cancer, kidney cancer, liver cancer, lung cancer, ovarian cancer, pancreatic cancer, skin cancer or thyroid cancer, in whom the levels or functions of ROR1 are absent or are decreased relative to a control or normal reference range. In further embodiments, compounds that promote the level or function of ROR1 are therapeutically or prophylactically administered to a subject suspected of having or known to have cancer e.g. bladder cancer, breast cancer, colorectal cancer, head and neck cancer, kidney cancer, liver cancer, lung cancer, ovarian cancer, pancreatic cancer, skin cancer or thyroid cancer in whom the levels or functions of ROR1 are increased relative to a control or to a reference range. In further embodiments, compounds that decrease the level or function of ROR1 are therapeutically or prophylactically administered to a subject suspected of having or known to have cancer e.g. bladder cancer, breast cancer, colorectal cancer, head and neck cancer, kidney cancer, liver cancer, lung cancer, ovarian cancer, pancreatic cancer, skin cancer or thyroid cancer in whom the levels or functions of ROR1 are increased relative to a control or to a reference range. In further embodiments, compounds that decrease the level or function of ROR1 are therapeutically or prophylactically administered to a subject suspected of having or known to have cancer e.g. bladder cancer, breast cancer, colorectal cancer, head and neck cancer, kidney cancer, liver cancer, lung cancer, ovarian cancer, pancreatic cancer, skin cancer or thyroid cancer in whom the levels or functions of ROR1 are decreased relative to a control or to a reference range. The change in ROR1 function or level due to the administration of such compounds can be readily detected, e.g. by obtaining a sample (e.g. blood or urine) and assaying in vitro the levels or activities of ROR1, or the levels of mRNAs encoding ROR1, or any combination of the foregoing. Such assays can be performed before and after the administration of the compound as described herein.

The compounds of the invention include but are not limited to any compound, e.g. a small organic molecule, protein, peptide, antibody (or other affinity reagent), nucleic acid, etc. that restores the ROR1 profile towards normal. The compounds of the invention may be given in combination with any other chemotherapy drugs.

Vaccine Therapy

Another aspect of the invention is an immunogenic composition, suitably a vaccine composition, comprising ROR1 or an epitope containing fragment thereof, or nucleic acid encoding ROR1 or a fragment thereof optionally together with an immunostimulant.

There is also provided a method of raising an immune response which comprises administering to a subject such compositions and a method for treating or preventing cancer e.g. bladder cancer, breast cancer, colorectal cancer, head and neck cancer, kidney cancer, liver cancer, lung cancer, ovarian cancer, pancreatic cancer, skin cancer or thyroid cancer which comprises administering to a subject in need thereof a therapeutically effective amount of such compositions and such compositions for use in preventing or treating bladder cancer, breast cancer, colorectal cancer, head and neck cancer, kidney cancer, liver cancer, lung cancer, ovarian cancer, pancreatic cancer, skin cancer or thyroid cancer.

Thus, ROR1 may be useful as antigenic material, and may be used in the production of vaccines for treatment or prophylaxis of cancer, e.g. bladder cancer, breast cancer, colorectal cancer, head and neck cancer, kidney cancer, liver cancer, lung cancer, ovarian cancer, pancreatic cancer, skin cancer or thyroid cancer. Such material can be “antigenic” and/or “immunogenic”. Generally, “antigenic” is taken to mean that the protein is capable of being used to raise antibodies (or other affinity reagents) or indeed is capable of inducing an antibody response in a subject or experimental animal “Immunogenic” is taken to mean that the protein is capable of eliciting an immune response such as a protective immune response in a subject or experimental animal. Thus, in the latter case, the protein may be capable of not only generating an antibody response but, in addition, non-antibody based immune responses. “Immunogenic” also embraces whether the protein may elicit an immune-like response in an in-vitro setting e.g. a T-cell proliferation assay. The generation of an appropriate immune response may require the presence of one or more adjuvants and/or appropriate presentation of an antigen.

The skilled person will appreciate that homologues or derivatives of ROR1 will also find use as antigenic/immunogenic material. Thus, for instance proteins which include one or more additions, deletions, substitutions or the like are encompassed by the present invention. In addition, it may be possible to replace one amino acid with another of similar “type”, for instance, replacing one hydrophobic amino acid with another. One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention.

In the case of homologues and derivatives, the degree of identity with a protein as described herein is less important than that the homologue or derivative should retain its antigenicity and/or immunogenicity. However, suitably, homologues or derivatives having at least 60% similarity (as discussed above) with the proteins or polypeptides described herein are provided, for example, homologues or derivatives having at least 70% similarity, such as at least 80% similarity are provided. Particularly, homologues or derivatives having at least 90% or even 95% similarity are provided. Suitably, homologues or derivatives have at least 60% sequence identity with the proteins or polypeptides described herein. Preferably, homologues or derivatives have at least 70% identity, more preferably at least 80% identity. Most preferably, homologues or derivatives have at least 90% or even 95% identity.

In an alternative approach, the homologues or derivatives could be fusion proteins, incorporating moieties which render purification easier, for example by effectively tagging the desired protein or polypeptide. It may be necessary to remove the “tag” or it may be the case that the fusion protein itself retains sufficient antigenicity to be useful.

It is well known that it is possible to screen an antigenic protein or polypeptide to identify epitopic regions, i.e. those regions which are responsible for the protein or polypeptide's antigenicity or immunogenicity. Methods well known to the skilled person can be used to test fragments and/or homologues and/or derivatives for antigenicity. Thus, the fragments of the present invention should include one or more such epitopic regions or be sufficiently similar to such regions to retain their antigenic/immunogenic properties. Thus, for fragments according to the present invention the degree of identity is perhaps irrelevant, since they may be 100% identical to a particular part of a protein or polypeptide, homologue or derivative as described herein. The key issue, once again, is that the fragment retains the antigenic/immunogenic properties of the protein from which it is derived.

What is important for homologues, derivatives and fragments is that they possess at least a degree of the antigenicity/immunogenicity of the protein or polypeptide from which they are derived. Thus, in an additional aspect of the invention, there is provided antigenic/or immunogenic fragments of ROR1, or of homologues or derivatives thereof.

ROR1, or antigenic fragments thereof, can be provided alone, as a purified or isolated preparation. They may be provided as part of a mixture with one or more other proteins of the invention, or antigenic fragments thereof. In a further aspect, therefore, the invention provides an antigen composition comprising ROR1 and/or one or more antigenic fragments thereof. Such a composition can be used for the detection and/or diagnosis of cancer, e.g. bladder cancer, breast cancer, colorectal cancer, head and neck cancer, kidney cancer, liver cancer, lung cancer, ovarian cancer, pancreatic cancer, skin cancer or thyroid cancer.

Vaccine compositions according to the invention may be either a prophylactic or therapeutic vaccine composition.

The vaccine compositions of the invention can include one or more adjuvants (immunostimulants). Examples well-known in the art include inorganic gels, such as aluminium hydroxide, and water-in-oil emulsions, such as incomplete Freund's adjuvant. Other useful adjuvants will be well known to the skilled person.

Suitable adjuvants for use in vaccine compositions for the treatment of cancer include: 3De-β-acylated monophosphoryl lipid A (known as 3D-MPL or simply MPL see WO92/116556), a saponin, for example QS21 or QS7, and TLR4 agonists such as a CpG containing molecule, for example as disclosed in WO95/26204. The adjuvants employed may be a combination of components, for example MPL and QS21 or MPL, QS21 and a CpG containing moiety. Adjuvants may be formulated as oil-in-water emulsions or liposomal formulations. Such preparations may include other vehicles.

In another embodiment, a preparation of oligonucleotides comprising 10 or more consecutive nucleotides complementary to a nucleotide sequence encoding ROR1 or a ROR1 peptide fragments is used as vaccines for the treatment of cancer, e.g. bladder cancer, breast cancer, colorectal cancer, head and neck cancer, kidney cancer, liver cancer, lung cancer, ovarian cancer, pancreatic cancer, skin cancer or thyroid cancer. Such preparations may include adjuvants or other vehicles.

Inhibition of ROR1 to Treat Bladder Cancer, Breast Cancer, Colorectal Cancer, Head and Neck Cancer, Kidney Cancer, Liver Cancer, Lung Cancer, Ovarian Cancer, Pancreatic Cancer, Skin Cancer or Thyroid Cancer

In one embodiment of the invention, cancer, e.g. bladder cancer, breast cancer, colorectal cancer, head and neck cancer, kidney cancer, liver cancer, lung cancer, ovarian cancer, pancreatic cancer, skin cancer or thyroid cancer is treated or prevented by administration of a compound that antagonizes (inhibits) the level and/or function of ROR1 which is elevated in the serum or tissue of subjects having such cancer as compared with serum or tissue of subjects free from such cancer.

Compounds useful for this purpose include but are not limited to anti-ROR1 antibodies (or other affinity reagents, and fragments and derivatives containing the binding region thereof), ROR1 antisense or ribozyme nucleic acids, and nucleic acids encoding dysfunctional ROR1 that may be used to “knockout” endogenous ROR1 function by homologous recombination (see, e.g. Capecchi, 1989, Science 244:1288-1292). Other compounds that inhibit ROR1 function can be identified by use of known in vitro assays, e.g. assays for the ability of a test compound to inhibit binding of ROR1 to another protein or a binding partner, or to inhibit a known ROR1 function.

Such inhibition may, for example, be assayed in vitro or in cell culture, but genetic assays may also be employed. The Preferred Technologies can also be used to detect levels of ROR1 before and after the administration of the compound. Suitable in vitro or in vivo assays are utilized to determine the effect of a specific compound and whether its administration is indicated for treatment of the affected tissue, as described in more detail below.

In a specific embodiment, a compound that inhibits ROR1 function (activity) is administered therapeutically or prophylactically to a subject in whom an increased serum or tissue level or functional activity of ROR1 (e.g. greater than the normal level or desired level) is detected as compared with serum or tissue of subjects with e.g. bladder cancer, breast cancer, colorectal cancer, head and neck cancer, kidney cancer, liver cancer, lung cancer, ovarian cancer, pancreatic cancer, skin cancer or thyroid cancer who do not receive treatment according to the invention or to bring the level or activity to that found in subjects free from such cancer, or a predetermined reference range. Methods standard in the art can be employed to measure the increase in ROR1 level or function, as outlined above. Suitable ROR1 inhibitor compositions may, for example, include small molecules, i.e. molecules of 1000 daltons or less. Such small molecules can be identified by the screening methods described herein.

Assays for Therapeutic or Prophylactic Compounds

The present invention also provides assays for use in drug discovery in order to identify or verify the efficacy of compounds for treatment or prevention of cancers expressing the extracellular domain of ROR1, e.g. bladder cancer, breast cancer, colorectal cancer, head and neck cancer, kidney cancer, liver cancer, lung cancer, ovarian cancer, pancreatic cancer, skin cancer or thyroid cancer.

Thus there is provided a method of screening for compounds that modulate the activity of ROR1, the method comprising: (a) contacting ROR1 or a biologically active portion thereof with a candidate compound; and (b) determining whether activity of ROR1 is thereby modulated. Such a process may comprise (a) contacting ROR1 or a biologically active portion thereof with a candidate compound in a sample; and (b) comparing the activity of ROR1 or a biologically active portion thereof in said sample after contact with said candidate compound with the activity of ROR1 or a biologically active portion thereof in said sample before contact with said candidate compound, or with a reference level of activity.

The method of screening may be a method of screening for compounds that inhibit activity of ROR1.

ROR1 or a biologically active portion thereof may, for example be expressed on or by a cell. ROR1 or a biologically active portion thereof may, for example, be isolated from cells which express it. ROR1 or a biologically active portion thereof may, for example, be immobilised onto a solid phase.

There is also provided a method of screening for compounds that modulate the expression of ROR1 or nucleic acid encoding ROR1, the method comprising: (a) contacting cells expressing ROR1 or nucleic acid encoding ROR1 with a candidate compound; and (b) determining whether expression of ROR1 or nucleic acid encoding ROR1 is thereby modulated. Such a process may comprises (a) contacting cells expressing ROR1 or nucleic acid encoding ROR1 with a candidate compound in a sample; and (b) comparing the expression of ROR1 or nucleic acid encoding ROR1 by cells in said sample after contact with said candidate compound with the expression of ROR1 or nucleic acid encoding ROR1 of cells in said sample before contact with said candidate compound, or with a reference level of expression.

The method may be a method of screening for compounds that inhibit expression of ROR1 or nucleic acid encoding ROR1.

Other aspects of the invention include: a compound obtainable by an aforementioned screening method, a compound which modulates the activity or expression of ROR1 or nucleic acid encoding ROR1, for example a compound which inhibits the activity or expression of ROR1 or nucleic acid encoding ROR1.

Such a compound is provided for use in treating or preventing cancer, e.g. bladder cancer, breast cancer, colorectal cancer, head and neck cancer, kidney cancer, liver cancer, lung cancer, ovarian cancer, pancreatic cancer, skin cancer or thyroid cancer. There is also provided a method for treating or preventing cancer, e.g. bladder cancer, breast cancer, colorectal cancer, head and neck cancer, kidney cancer, liver cancer, lung cancer, ovarian cancer, pancreatic cancer, skin cancer or thyroid cancer which comprises administering to a subject in need thereof a therapeutically effective amount of such a compound.

Test compounds can be assayed for their ability to restore ROR1 levels in a subject having e.g. bladder cancer, breast cancer, colorectal cancer, head and neck cancer, kidney cancer, liver cancer, lung cancer, ovarian cancer, pancreatic cancer, skin cancer or thyroid cancer towards levels found in subjects free from such cancers or to produce similar changes in experimental animal models of such cancers. Compounds able to restore ROR1 levels in a subject having e.g. bladder cancer, breast cancer, colorectal cancer, head and neck cancer, kidney cancer, liver cancer, lung cancer, ovarian cancer, pancreatic cancer, skin cancer or thyroid cancer towards levels found in subjects free from such cancers or to produce similar changes in experimental animal models of such cancers can be used as lead compounds for further drug discovery, or used therapeutically. ROR1 expression can be assayed by the Preferred Technologies, immunoassays, gel electrophoresis followed by visualization, detection of ROR1 activity, or any other method taught herein or known to those skilled in the art. Such assays can be used to screen candidate drugs, in clinical monitoring or in drug development, where abundance of ROR1 can serve as a surrogate marker for clinical disease.

In various specific embodiments, in vitro assays can be carried out with cells representative of cell types involved in a subject's disorder, to determine if a compound has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used. Examples of animal models of bladder cancer, breast cancer, colorectal cancer, head and neck cancer, kidney cancer, liver cancer, lung cancer, ovarian cancer, pancreatic cancer, skin cancer or thyroid cancer include, but are not limited to xenografts of bladder cancer cell lines such as UCRU-BL-12, UCRU-BL-13 and UCRU-BL-14, Russell et al. Cancer Res. 1986 April; 46(4 Pt 2):2035-40; xenografts of breast cancer cell lines such as MCF-7 (Ozzello L, Sordat M., Eur J Cancer. 1980; 16:553-559) and MCF10AT (Miller et al., J Natl Cancer Inst. 1993; 85:1725-1732) in nude or SCID mice; xenografts of human colorectal cancer cell lines such as MDA-MB-345 in oestrogen-deprived SCID mice, Eccles et al. 1994 Cell Biophysics 24/25, 279; xenografts of head and neck cancer cell lines such as FaDu and HNX-OE; xenografts of liver cancer cell lines such as MHCC97 in nude mice, Tian et al., Br J. Cancer 1999 November; 81(5):814-21; xenografts of non small cell lung cancer cell lines such as A549 and H460 and xenografts of small cell lung cancer cell lines such as NCI-H345; xenografts of ovarian cancer cell lines such as IGROV1 in nude mice, Benard et al, Cancer Res. 1985 October; 45(10):4970-9; xenografts of pancreatic cancer cell lines such as MIA PaCa-2 in nude mice, Marincola et al., J Surg Res 1989 December; 47(6):520-9; xenografts of skin cancer cell lines such as MV3 in nude mice, van Muijen et al., Int J Cancer 1991 Apr. 22; 48(1):85-91 or xenografts of thyroid cancer cell lines such as ARO, Viaggi et al., Thyroid 2003 June; 13(6):529-36. These can be utilized to test compounds that modulate ROR1 levels, since the pathology exhibited in these models is similar to that of e.g. bladder cancer, breast cancer, colorectal cancer, head and neck cancer, kidney cancer, liver cancer, lung cancer, ovarian cancer, pancreatic cancer, skin cancer or thyroid cancer. It is also apparent to the skilled artisan that based upon the present disclosure, transgenic animals can be produced with “knock-out” mutations of the gene or genes encoding ROR1. A “knock-out” mutation of a gene is a mutation that causes the mutated gene to not be expressed, or expressed in an aberrant form or at a low level, such that the activity associated with the gene product is nearly or entirely absent. Preferably, the transgenic animal is a mammal; more preferably, the transgenic animal is a mouse.

In one embodiment, test compounds that modulate the expression of ROR1 are identified in non-human animals (e.g. mice, rats, monkeys, rabbits, and guinea pigs), preferably non-human animal models for bladder cancer, breast cancer, colorectal cancer, head and neck cancer, kidney cancer, liver cancer, lung cancer, ovarian cancer, pancreatic cancer, skin cancer or thyroid cancer expressing ROR1. In accordance with this embodiment, a test compound or a control compound is administered to the animals, and the effect of the test compound on expression of ROR1 is determined. A test compound that alters the expression of ROR1 can be identified by comparing the level of ROR1 (or mRNA encoding the same) in an animal or group of animals treated with a test compound with the level of ROR1 or mRNA in an animal or group of animals treated with a control compound. Techniques known to those of skill in the art can be used to determine the mRNA and protein levels, for example, in situ hybridization. The animals may or may not be sacrificed to assay the effects of a test compound.

In another embodiment, test compounds that modulate the activity of ROR1 or a biologically active portion thereof are identified in non-human animals (e.g. mice, rats, monkeys, rabbits, and guinea pigs), preferably non-human animal models for bladder cancer, breast cancer, colorectal cancer, head and neck cancer, kidney cancer, liver cancer, lung cancer, ovarian cancer, pancreatic cancer, skin cancer or thyroid cancer expressing ROR1. In accordance with this embodiment, a test compound or a control compound is administered to the animals, and the effect of a test compound on the activity of ROR1 is determined. A test compound that alters the activity of ROR1 can be identified by assaying animals treated with a control compound and animals treated with the test compound. The activity of ROR1 can be assessed by detecting induction of a cellular second messenger of ROR1 (e.g. intracellular Ca²⁺, diacylglycerol, IP3, etc.), detecting catalytic or enzymatic activity of ROR1 or binding partner thereof, detecting the induction of a reporter gene (e.g. a regulatory element that is responsive to ROR1 operably linked to a nucleic acid encoding a detectable marker, such as luciferase or green fluorescent protein), or detecting a cellular response (e.g. cellular differentiation or cell proliferation). Techniques known to those of skill in the art can be utilized to detect changes in the activity of ROR1 (see, e.g. U.S. Pat. No. 5,401,639, which is incorporated herein by reference).

In yet another embodiment, test compounds that modulate the level or expression of ROR1 are identified in human subjects having e.g. bladder cancer, breast cancer, colorectal cancer, head and neck cancer, kidney cancer, liver cancer, lung cancer, ovarian cancer, pancreatic cancer, skin cancer or thyroid cancer, preferably those having e.g. severe bladder cancer, breast cancer, colorectal cancer, head and neck cancer, kidney cancer, liver cancer, lung cancer, ovarian cancer, pancreatic cancer, skin cancer or thyroid cancer. In accordance with this embodiment, a test compound or a control compound is administered to the human subject, and the effect of a test compound on ROR1 expression is determined by analyzing the expression of ROR1 or the mRNA encoding the same in a biological sample (e.g. serum, plasma, or urine). A test compound that alters the expression of ROR1 can be identified by comparing the level of ROR1 or mRNA encoding the same in a subject or group of subjects treated with a control compound to that in a subject or group of subjects treated with a test compound. Alternatively, alterations in the expression of ROR1 can be identified by comparing the level of ROR1 or mRNA encoding the same in a subject or group of subjects before and after the administration of a test compound. Techniques known to those of skill in the art can be used to obtain the biological sample and analyze the mRNA or protein expression. For example, the Preferred Technologies described herein can be used to assess changes in the level of ROR1.

In another embodiment, test compounds that modulate the activity of ROR1 are identified in human subjects having e.g. bladder cancer, breast cancer, colorectal cancer, head and neck cancer, kidney cancer, liver cancer, lung cancer, ovarian cancer, pancreatic cancer, skin cancer or thyroid cancer (preferably those with e.g. severe bladder cancer, breast cancer, colorectal cancer, head and neck cancer, kidney cancer, liver cancer, lung cancer, ovarian cancer, pancreatic cancer, skin cancer or thyroid cancer). In this embodiment, a test compound or a control compound is administered to the human subject, and the effect of a test compound on the activity of ROR1 is determined A test compound that alters the activity of ROR1 can be identified by comparing biological samples from subjects treated with a control compound to samples from subjects treated with the test compound. Alternatively, alterations in the activity of ROR1 can be identified by comparing the activity of ROR1 in a subject or group of subjects before and after the administration of a test compound. The activity of ROR1 can be assessed by detecting in a biological sample (e.g. serum, plasma, or urine) induction of a cellular signal transduction pathway of ROR1 (e.g. intracellular Ca²⁺, diacylglycerol, IP3, etc.), catalytic or enzymatic activity of ROR1 or a binding partner thereof, or a cellular response, for example, cellular differentiation, or cell proliferation. Techniques known to those of skill in the art can be used to detect changes in the induction of a second messenger of ROR1 or changes in a cellular response. For example, RT-PCR can be used to detect changes in the induction of a cellular second messenger.

In another embodiment, a test compound that changes the level or expression of ROR1 towards levels detected in control subjects (e.g. humans free from e.g. bladder cancer, breast cancer, colorectal cancer, head and neck cancer, kidney cancer, liver cancer, lung cancer, ovarian cancer, pancreatic cancer, skin cancer and thyroid cancer) is selected for further testing or therapeutic use. In another embodiment, a test compound that changes the activity of ROR1 towards the activity found in control subjects (e.g. humans free from e.g. bladder cancer, breast cancer, colorectal cancer, head and neck cancer, kidney cancer, liver cancer, lung cancer, ovarian cancer, pancreatic cancer, skin cancer and thyroid cancer) is selected for further testing or therapeutic use.

In another embodiment, test compounds that reduce the severity of one or more symptoms associated with e.g. bladder cancer, breast cancer, colorectal cancer, head and neck cancer, kidney cancer, liver cancer, lung cancer, ovarian cancer, pancreatic cancer, skin cancer or thyroid cancer are identified in human subjects having e.g. bladder cancer, breast cancer, colorectal cancer, head and neck cancer, kidney cancer, liver cancer, lung cancer, ovarian cancer, pancreatic cancer, skin cancer or thyroid cancer, preferably subjects with e.g. severe bladder cancer, breast cancer, colorectal cancer, head and neck cancer, kidney cancer, liver cancer, lung cancer, ovarian cancer, pancreatic cancer, skin cancer or thyroid cancer. In accordance with this embodiment, a test compound or a control compound is administered to the subjects, and the effect of a test compound on one or more symptoms of e.g. bladder cancer, breast cancer, colorectal cancer, head and neck cancer, kidney cancer, liver cancer, lung cancer, ovarian cancer, pancreatic cancer, skin cancer or thyroid cancer is determined. A test compound that reduces one or more symptoms can be identified by comparing the subjects treated with a control compound to the subjects treated with the test compound. Techniques known to physicians familiar with e.g. bladder cancer, breast cancer, colorectal cancer, head and neck cancer, kidney cancer, liver cancer, lung cancer, ovarian cancer, pancreatic cancer, skin cancer or thyroid cancer can be used to determine whether a test compound reduces one or more symptoms associated with e.g. bladder cancer, breast cancer, colorectal cancer, head and neck cancer, kidney cancer, liver cancer, lung cancer, ovarian cancer, pancreatic cancer, skin cancer or thyroid cancer. For example, a test compound that reduces tumour burden in a subject having e.g. bladder cancer, breast cancer, colorectal cancer, head and neck cancer, kidney cancer, liver cancer, lung cancer, ovarian cancer, pancreatic cancer, skin cancer or thyroid cancer will be beneficial for such subject.

In another embodiment, a test compound that reduces the severity of one or more symptoms associated with cancer, e.g. bladder cancer, breast cancer, colorectal cancer, head and neck cancer, kidney cancer, liver cancer, lung cancer, ovarian cancer, pancreatic cancer, skin cancer or thyroid cancer in a human having bladder cancer, breast cancer, colorectal cancer, head and neck cancer, liver cancer, lung cancer, ovarian cancer, pancreatic cancer, skin cancer or thyroid cancer is selected for further testing or therapeutic use.

Therapeutic and Prophylactic Compositions and their Use

The invention provides methods of treatment (and prophylaxis) comprising administering to a subject an effective amount of a compound of the invention. In a particular aspect, the compound is substantially purified (e.g. substantially free from substances that limit its effect or produce undesired side-effects). The subject is for example an animal, including but not limited to animals such as cows, pigs, horses, chickens, cats, dogs, etc., and is for example a mammal, such as a human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g. encapsulation in liposomes, microparticles, microcapsules, recombinant cells capable of expressing the compound, receptor-mediated endocytosis (see, e.g. Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g. oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir Pulmonary administration can also be employed, e.g. by use of an inhaler or nebulizer, and formulation with an aerosolizing agent.

In one aspect of the invention a nucleic acid employed in the invention may be delivered to the dermis, for example employing particle mediated epidermal delivery.

In a specific embodiment, it may be desirable to administer the pharmaceutical compositions of the invention locally to the area in need of treatment; this may be achieved, for example, and not by way of limitation, by local infusion during surgery, topical application, e.g. by injection, by means of a catheter, or by means of an implant, said implant being of a porous, non-porous, or gelatinous material, including membranes, such as sialastic membranes, or fibers. In one embodiment, administration can be by direct injection into e.g. bladder, breast, colorectal, head and neck, kidney, liver, lung, ovarian, pancreatic, skin or thyroid tissue or at the site (or former site) of a malignant tumor or neoplastic or pre-neoplastic tissue.

In another embodiment, the compound can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton, 1987, CRC Crit. Ref Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, e.g. the bladder, breast, colon, head and neck, kidney, liver, lung, ovary, pancreas, skin or thyroid thus requiring only a fraction of the systemic dose (see, e.g. Goodson, in Medical Applications of Controlled Release, supra, vol. 2, pp. 115-138 (1984)). Other controlled release systems are discussed in the review by Langer (1990, Science 249:1527-1533).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g. by use of a retroviral vector (see U.S. Pat. No. 4,980,286), or by direct injection, or by use of microparticle bombardment (e.g. a gene gun; Biolistic, Dupont), or coating with lipids or cell-surface receptors or transfecting agents, or by administering it in linkage to a homeobox-like peptide which is known to enter the nucleus (see e.g. Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means suitable for approval by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, for example in purified form, together with a suitable amount of carrier so as to provide the form for proper administration to the subject. The formulation should suit the mode of administration.

In one embodiment, for example where one or more antibodies are employed, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts, where appropriate, include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of cancer, for example, bladder cancer, breast cancer, colorectal cancer, head and neck cancer, kidney cancer, liver cancer, lung cancer, ovarian cancer, pancreatic cancer, skin cancer or thyroid cancer can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Thus in one aspect the kit comprises antibodies employed in the invention, for example the antibodies may be lyophilized for reconstitution before administration or use. Where the kit is for use in therapy/treatment such as cancer the antibody or antibodies may be reconstituted with an isotonic aqueous solution, which may optionally be provided with the kit. In one aspect the kit may comprise a polypeptide such as an immunogenic polypeptide employed in the invention, which may for example be lyophilized. The latter kit may further comprise an adjuvant for reconstituting the immunogenic polypeptide.

The invention also extends to a composition as described herein for example a pharmaceutical composition and/or vaccine composition for use in inducing an immune response in a subject.

Determining Abundance of ROR1 by Imaging Technology

An advantage of determining abundance of ROR1 by imaging technology may be that such a method is non-invasive (save that reagents may need to be administered) and there is no need to extract a sample from the subject.

Suitable imaging technologies include positron emission tomography (PET) and single photon emission computed tomography (SPECT). Visualisation of ROR1 using such techniques requires incorporation or binding of a suitable label e.g. a radiotracer such as ¹⁸F, ¹¹C or ¹²³I (see e.g. NeuroRx—The Journal of the American Society for Experimental NeuroTherapeutics (2005) 2(2), 348-360 and idem pages 361-371 for further details of the techniques). Radiotracers or other labels may be incorporated into ROR1 by administration to the subject (e.g. by injection) of a suitably labelled specific ligand. Alternatively they may be incorporated into a binding affinity reagent (e.g. antibody) specific for ROR1 which may be administered to the subject (e.g. by injection). For discussion of use of Affibodies for imaging see e.g. Orlova A, Magnusson M, Eriksson TL, Nilsson M, Larsson B, Hoiden-Guthenberg I, Widstrom C, Carlsson J, Tolmachev V, Stahl S, Nilsson FY, Tumor imaging using a picomolar affinity HER2 binding Affibody molecule, Cancer Res. 2006 Apr. 15; 66(8):4339-48).

Diagnosis and Treatment of Cancer Including Bladder Cancer, Breast Cancer, Colorectal Cancer, Head and Neck Cancer, Kidney Cancer, Liver Cancer, Lung Cancer, Ovarian Cancer, Pancreatic Cancer, Skin Cancer or Thyroid Cancer Using Immunohistochemistry

Immunohistochemistry is an excellent detection technique and may therefore be very useful in the diagnosis and treatment of cancer, including bladder cancer, breast cancer, colorectal cancer, head and neck cancer, kidney cancer, liver cancer, lung cancer, ovarian cancer, pancreatic cancer, skin cancer or thyroid cancer. Immunohistochemistry may be used to detect, diagnose, or monitor cancers such as those mentioned above, through the localization of ROR1 antigens in tissue sections by the use of labeled antibodies (or other affinity reagents), derivatives and analogs thereof, which specifically bind to ROR1, as specific reagents through antigen-antibody interactions that are visualized by a marker such as fluorescent dye, enzyme, radioactive element or colloidal gold.

The advancement of monoclonal antibody technology has been of great significance in assuring the place of immunohistochemistry in the modern accurate microscopic diagnosis of human neoplasms. The identification of disseminated neoplastically transformed cells by immunohistochemistry allows for a clearer picture of cancer invasion and metastasis, as well as the evolution of the tumour cell associated immunophenotype towards increased malignancy. Future antineoplastic therapeutical approaches may include a variety of individualized immunotherapies, specific for the particular immunophenotypical pattern associated with each individual patient's neoplastic disease. For further discussion see e.g. Bodey B, The significance of immunohistochemistry in the diagnosis and therapy of neoplasms, Expert Opin Biol Ther. 2002 April; 2(4):371-93.

Further background information regarding examples of cancer types which may be treated or diagnosed according to the present invention include the following:

Bladder Cancer—In the United States, bladder cancer is the fourth most common type of cancer in men and the ninth most common cancer in women. More than 51,000 men and 17,000 women are diagnosed with bladder cancer each year, with around 14,000 deaths in total. One reason for its higher incidence in men is that the androgen receptor, which is much more active in men than in women, plays a major part in the development of the cancer. Incidence of bladder cancer increases with age. People over the age of 70 develop the disease 2 to 3 times more often than those aged 55-69 and 15 to 20 times more often than those aged 30-54. Bladder cancer is 2 to 3 times more common in men. Smoking is a major contributory factor, accounting for up to 65 percent of cases in men and 30 percent of cases in women in developed countries. It has been estimated that approximately US$2 billion is spent in the United States on treating bladder cancer. The NCI's investment in bladder cancer research has increased from US$19.1 million in 2000 to an estimated US$34.8 million in 2005. Most patients when first diagnosed with bladder cancer have their cancer confined to the bladder (74%). In 19% of the cases, the cancer has spread to nearby tissues outside the bladder and in 3% it has spread to distant sites. Bladder cancer can be diagnosed using cystoscopy, biopsy, urine cytology and imaging tests such as an intravenous pyelogram (IVP), computed tomography (CT) scan, magnetic resonance imaging (MRI) scan or ultrasound. Bladder cancer is staged using the American Joint Committee on Cancer (AJCC) TNM system—stage I-IV. The main types of treatment for bladder cancer are surgery, radiation therapy, immunotherapy and chemotherapy. Surgery, alone or combined with other treatments, is used in more than 90% of cases. For early stage or superficial bladder cancer, a transurethral resection (TUR) is most common. About 70-80% of patients have superficial cancer when first diagnosed. When the bladder cancer is invasive, a cystectomy is sometimes necessary. An alternative approach for locally advanced bladder cancer can be a TUR along with radiation therapy and chemotherapy. Bacillus Calmette-Guerin (BCG) can be used as immunotherapy for treating low-stage bladder cancer. Neoadjuvant or adjuvant chemotherapy can be used in the treatment of bladder cancer. Mitomycin and thiotepa are the drugs most often used for intravesical chemotherapy. Systemic chemotherapy combinations used to treat bladder cancer include M-VAC (methotrexate, vinblastine, doxorubicin and cisplatin), MCV (methotrexate, cisplatin and vinblastine) and GemCIS (gemcitabine and cisplatin). External beam radiation therapy or local or interstitial radiation therapy can be combined with chemotherapy after surgery. Patients diagnosed with bladder cancer have a 5 year relative survival rate of 95% for stage 0, 85% for stage I, 55% for stage II, 38% for stage III and 16% for stage IV.

Breast Cancer—Globally, breast cancer is both the most common cancer (10% of all cancer cases) and the leading cause of cancer death (6% of cancer deaths) in women. Global incidence of breast cancer is over 1 million cases per year, with about 400,000 deaths. Women in North America have the highest rate of breast cancer in the world (over 200,000 new cases per year, with about 40,000 deaths). The chance of developing invasive breast cancer at some time in a woman's life is about 1 in 8. Breast cancer incidence increases with age, rising sharply after age 40. In the USA, about 77% of invasive breast cancers occur in women over age 50. It has been estimated that approximately US$8.1 billion is spent in the USA each year on treating breast cancer. Early diagnosis improves the likelihood that treatment will be successful. Screening methods such as mammograms, clinical breast examinations and breast self-examinations are useful in detecting breast cancer. Current diagnostic methods include breast ultrasound, ductogram, full-field digital mammography (FFDM), scintimammography and MRI. A biopsy (fine needle aspiration biopsy, core biopsy or surgical biopsy) is then performed to confirm the presence of breast cancer. Imaging tests such as a chest x-ray, bone scan, CT, MRI and PET are used to detect if the breast cancer has spread. Breast cancer is staged using the American Joint Committee on Cancer (AJCC) TNM system—Stage 0-Stage IV. Ductal carcinoma in situ (DCIS), a non-invasive cancer which accounts for 20% of new breast cancer cases is Stage 0. Nearly all women diagnosed at this early stage of breast cancer can be cured. Infiltrating (invasive) ductal carcinoma (IDC), which accounts for 80% of invasive breast cancer and infiltrating (invasive) lobular carcinoma (ILC), which accounts for 5% of invasive breast cancers are more severe Stage I-IV cancers and can metastasize. Breast-conserving surgery (lumpectomy) or mastectomy are the usual treatments for breast cancer. For stage I or II breast cancer, breast-conserving surgery is as effective as mastectomy. Patients can then undergo reconstructive surgery. Axillary lymph node sampling and removal or sentinel lymph node biopsy (SLNB) is performed to see if the cancer has spread to the lymph nodes. Neoadjuvant chemotherapy can be given before surgery to shrink large cancers. Adjuvant chemotherapy after surgery reduces the risk of breast cancer recurrence. Chemotherapy can also be used as the main treatment for women whose cancer has spread outside the breast and underarm area. Chemotherapeutic agents used include anthracyclines (e.g. methotrexate, fluorouracil, doxorubicin, epirubicin), taxanes (e.g. paclitaxel, docetaxel, vinorelbine) and alkylating agents (e.g. cyclophosphamide). Radiation therapy (usually external beam radiation but sometimes brachytherapy) is given once chemotherapy is complete. Hormone therapy with selective estrogen receptor modulators (e.g. tamoxifen) can be given to women with estrogen receptor positive breast cancers. Taking tamoxifen after surgery for 5 years can reduce recurrence by about 50% in women with early breast cancer. Aromatase inhibitors such as exemestane, letrozole or anastrozole can also be used. Women with HER2 positive cancers (about ⅓ of breast cancers) can be given biological response modifiers such as trastuzumab (Herceptin). Clinical trials have shown that adding trastuzumab to chemotherapy lowers the recurrence rate and death rate over chemotherapy alone after surgery in women with HER2 positive early breast cancers. Patients diagnosed with breast cancer between 1995 and 1998 had a 5 year relative survival rate of 100% for stage 0 and I, 92% for stage IIA, 81% for stage IIB, 67% for stage IIIA, 54% for stage IIIB and 20% for stage IV.

Colorectal Cancer (CRC)—CRC is one of the leading causes of cancer-related morbidity and mortality, responsible for an estimated half a million deaths per year, mostly in Western, well developed countries. In these territories, CRC is the third most common malignancy (estimated number of new cases per annum in USA and EU is approximately 350,000 per year). Estimated healthcare costs related to treatment for colorectal cancer in the United States are more than $8 billion. Today, the fecal occult blood test and colonoscopy, a highly invasive procedure, are the most frequently used screening and diagnostic methods for colorectal cancer. Other diagnostic tools include Flexible Sigmoidoscopy (allowing the observation of only about half of the colon) and Double Contrast Barium Enema (DCBE, to obtain X-ray images). CRC has four distinct stages: patients with stage I disease have a five-year survival rate of >90%, while those with metastatic stage IV disease have a <5% survival rate according to the US National Institutes of Health (NIH). Once CRC has been diagnosed, the correct treatment needs to be selected. Surgery is usually the main treatment for rectal cancer, although radiation and chemotherapy will often be given before surgery. Possible side effects of surgery include bleeding from the surgery, deep vein thrombosis and damage to nearby organs during the operation.

Currently, 60 percent of colorectal cancer patients receive chemotherapy to treat their disease; however, this form of treatment only benefits a few percent of the population, while carrying with it high risks of toxicity, thus demonstrating a need to better define the patient selection criteria.

Colorectal cancer has a 30 to 40 percent recurrence rate within an average of 18 months after primary diagnosis. As with all cancers, the earlier it is detected the more likely it can be cured, especially as pathologists have recognised that the majority of CRC tumours develop in a series of well-defined stages from benign adenomas. For stage I 93%, for stage HA 85%, for stage IIB 72%, for stage IIIA 83%, for stage IIIB 64%, for stage IIIC 44% and for stage IV 8%.

Head and Neck Cancer—The term head and neck cancer refers to a group of biologically similar cancers originating from the upper aerodigestive tract, including the lip, oral cavity (mouth), nasal cavity, paranasal sinuses, pharynx, and larynx. Most head and neck cancers are squamous cell carcinomas, originating from the mucosal lining (epithelium) of these regions. Head and neck cancers often spread to the lymph nodes of the neck, and this is often the first manifestation of the disease at the time of diagnosis. The number of new cases of head and neck cancers in the United States was 40,490 in 2006, accounting for about 3% of adult malignancies. 11,170 patients died of their disease in 2006. The worldwide incidence exceeds half a million cases annually. 85% of head and neck cancers are linked to tobacco use. In North America and Europe, the tumours usually arise from the oral cavity, oropharynx, or larynx, whereas nasopharyngeal cancer is more common in the Mediterranean countries and in the Far East. In Southeast China and Taiwan, head and neck cancer, specifically nasopharyngeal cancer is the most common cause of death in young men. African Americans are disproportionately affected by head and neck cancer, with younger ages of incidence, increased mortality, and more advanced disease at presentation. Head and neck cancer is diagnosed using a combination of tests which can include a physical examination, endoscopy, X-ray, computed tomography (CT) scan, magnetic resonance imaging (MRI) scan, PET scan and a biopsy. Early signs of head and neck cancer are often not detected and the majority of head and neck cancer patients present with advanced disease and often have secondary tumours. Head and neck cancer is staged using the American Joint Committee on Cancer (AJCC) TNM system—stage I-IV. The 5-year survival for all stages of head and neck cancer is 35-50%, due, in part, to late presentation. Stage I and II survival rates range from 40-95% and stage III and IV survival rates range from 0-50%. It is predicted that at least one third of patients with head and neck cancer will ultimately die as a result of their disease. The 5-year mortality rate has not altered significantly in the last few decades, despite advances in treatment modalities. Surgery and radiation therapy are the primary modalities of therapy, often in combination. Chemotherapy can be used as an induction therapy or as an adjuvant to radiation therapy, with or without surgery.

Liver Cancer—Around 80% of all cases of liver cancer is hepatocellular carcinoma (HCC), which arises from the main cells of the liver (the hepatocytes). It is usually confined to the liver and is associated with cirrhosis in 50% to 80% of patients. Hepatocellular carcinoma is about 3 times more common in males than in females. Chronic infection with hepatitis B virus (HBV) or hepatitis C virus (HCV) is a major cause of HCC and is responsible for making liver cancer the most common cancer in many parts of the world. In the United States, hepatitis C infection is responsible for about 50% to 60% of all liver cancers and hepatitis B is responsible for another 20%. Exposure to Aflatoxins is also a cause of HCC, mostly in warmer and tropical countries. Liver cancer accounts for about 5.8% of all cancer cases globally (about 626,000 cases) and 8.9% of deaths per year (about 598,000). It is the 3rd most common cause of cancer-related death in both men and women worldwide. HCC is predominantly found in Asia and Africa, which account for 80% of cases. In the USA, there are approximately 18,500 new cases of HCC and 16,000 deaths per year. About 85% of people diagnosed with liver cancer are between 45 and 85 years of age. About 4% are between 35 and 44 years of age and only 2.4% are younger than 35. Since symptoms of liver cancer often do not appear until the disease is advanced, only a small number of liver cancers are found in the early stages and can be removed with surgery.

Many signs and symptoms of liver cancer are relatively nonspecific—that is, they can be caused by other cancers or by non-cancerous diseases. Imaging tests such as ultrasound, computed tomography (CT), magnetic resonance imaging (MRI) and angiography are commonly used to diagnose HCC. Other diagnostic tools include laparoscopy, biopsy, alpha-fetoprotein (AFP) blood test, liver function tests (LFTs), prothrombin time (PT) and tests for hepatitis B and C. Liver cancer has four stages, stage Ito stage IV according to the American Joint Committee on Cancer (AJCC) TNM system. HCC can be classified as localized resectable, localized unresectable or advanced. The overall 5-year relative survival rate for liver cancer is about 9%. One reason for this low survival rate is that most patients with liver cancer also have cirrhosis of the liver, which itself can be fatal (people with liver cancer and class C cirrhosis are generally too sick for any treatment and usually die in a few months). The 5 year survival for localized resectable HCC following surgery is between 40% and 70%. For advanced HCC there is no standard treatment and the 5 year survival rate is less than 5%. Survival continues to drop after diagnosis and treatment so that by 10 years it is less than 2.5%. Treatment of liver cancer depends on the size of the tumour and whether the patient has cirrhosis. At this time, surgery, either by resection or liver transplantation, offers the only chance to cure a liver cancer. People without cirrhosis can do well with surgical removal of the tumour. However, in many cases, it might not be possible to safely remove a localized liver cancer. Less than 30% of the patients having explorative surgery are able to have their cancer completely removed by surgery. Partial hepatectomy results in a 5-year survival of 30% to 40%. If there is cirrhosis, or a very large tumour, most experts recommend liver transplantation as the main treatment. The 5-year survival for liver transplantation patients is around 70% but the opportunities for liver transplantation are limited. Other treatments include radiofrequency ablation (RFA), ethanol ablation, cryosurgery, hepatic artery embolization, chemoembolization or three-dimensional conformal radiation therapy (3DCRT). Chemotherapy can also be used but shrinks fewer than 1 in 5 tumours. This may be improved by hepatic artery infusion (HAI). Chemotherapeutic agents used include Adriamycin, VP-16, Cisplatinum, Mitomycin, 5-FU and Leucovorin. The prognosis for any treated primary liver cancer patient with progressing, recurring, or relapsing disease is poor. Treatment of liver cancer that returns after initial therapy depends on many factors, including the site of the recurrence, the type of initial treatment, and the functioning of the liver. Patients with localized resectable disease that recurs in the same spot may be eligible for further surgery.

Lung Cancer—Lung cancer is the most common form of cancer worldwide (accounting for about 12% of cancer cases) and the main cause of death from cancer (accounting for about 18% of deaths).

Global incidence of lung cancer is over 1,300,000 per year, with the number of deaths over 1,100,000. In the USA, there are about 170,000 new cases per year (about 13% of all cancers), with about 160,000 deaths (about 28% of cancer deaths). Lung cancer is much more prevalent among men than women. Nearly 70% of people diagnosed with lung cancer are older than 65; fewer than 3% of all cases are found in people under the age of 45. Around 15% of all lung cancers are small cell type (SCLC), which tend to spread widely through the body, while the remaining 85% are non-small cell (NSCLC). It has been estimated that approximately US$9.6 billion is spent in the USA each year on treating lung cancer. Lung cancer is a life-threatening disease because it often metastasises even before it can be detected on a chest x-ray. Usually symptoms of lung cancer do not appear until the disease is in an advanced stage. So far, there is no screening test that has been shown to improve a person's chance for a cure. Imaging tests such as a chest x-ray, CT scan, MRI scan or PET scan may be used to detect lung cancer. Tests to confirm the diagnosis are then performed and include sputum cytology, needle biopsy, bronchoscopy, endobronchial ultrasound and complete blood count (CBC). Nearly 60% of people diagnosed with lung cancer die within one year of diagnosis; 75% die within 2 years. The 5-year survival rate for people diagnosed with NSCLC is about 15%; for SCLC the 5-year survival rate is about 6%. NSCLC is staged using the American Joint Committee on Cancer (AJCC) TNM system—Stage 0-Stage IV. The 5-year survival rates by stage are as follows: stage I: 47%; stage II; 26%; stage III: 8% and stage IV: 2%. SCLC has a 2-stage system—limited stage and extensive stage. About two thirds of SCLC patients have extensive disease at diagnosis. If SCLC is found very early and is localised to the lung alone, the 5-year survival rate is around 21%, but only 6% of patients fall into this category. Where the cancer has spread, the 5-year survival is around 11%. For patients with extensive disease, the 5-year survival is just 2%. Surgery is the only reliable method to cure NSCLC. Types of surgery include lobectomy, pneumonectomy, segmentectomy and video-assisted thoracic surgery (for small tumours). External beam radiation therapy is sometimes used as the primary treatment, especially if the patient's health is too poor to undergo surgery. Radiation therapy can also be used after surgery. Chemotherapy may be given as the primary treatment or as an adjuvant to surgery. Targeted therapy using epidermal growth factor receptor (EGFR) antagonists such as gefitinib or erlotinib can also be given after other treatments have failed. Antiangiogenic drugs, such as bevacizumab, have been found to prolong survival of patients with advanced lung cancer. Photodynamic therapy is also being researched as a treatment for lung cancer. The main treatment for SCLC is chemotherapy, either alone or in combination with external beam radiation therapy and very rarely, surgery. Chemotherapeutic agents used for NSCLC and SCLC include cisplatin, carboplatin, mitomycin C, ifosfamide, vinblastine, gemcitabine, etoposide, vinorelbine, paclitaxel, docetaxel and irinotecan.

Ovarian Cancer—Ovarian cancer accounts for about 1.9% of cancer cases globally and around 1.8% of deaths. Global incidence of ovarian cancer is around 205,000, predominantly in post-menopausal women in developed countries, with around 125,000 deaths. About 85% to 90% of ovarian cancers are epithelial ovarian carcinomas. About 5% of ovarian cancers are germ cell tumours and a smaller percentage are stromal tumours. Ovarian cancer is the eighth most common cancer among women. In the USA, about 20,200 new cases of ovarian cancer are diagnosed each year and it accounts for about 3% of all cancers in women. The risk of developing and dying from ovarian cancer is higher for white women than black women. Around two-thirds of women with ovarian cancer are 55 or older. Ovarian cancer ranks fifth in cancer deaths among women in the USA, accounting for more deaths than any other cancer of the female reproductive system. There are around 15,300 deaths in the USA from ovarian cancer each year. It has been estimated that approximately US$2.2 billion is spent in the USA each year on treating ovarian cancer. It is currently difficult to diagnose ovarian cancer at an early stage. Imaging tests such as ultrasound, computed tomography and magnetic resonance imaging can confirm whether a pelvic mass is present. Blood tests, including a CA-125 test and a laparoscopy are performed. Ovarian cancer is then confirmed by biopsy. Ovarian cancer is staged using the American Joint Committee on Cancer (AJCC) TNM system—stage I-IV. The FIGO (International Federation of Gynecology and Obstetrics) system is also used. Ovarian cancers are also given a grade from 1-3. About 76% of women with ovarian cancer survive 1 year after diagnosis, and 45% survive longer than 5 years after diagnosis. If diagnosed and treated while the cancer has not spread outside the ovary, the 5-year survival rate is 94%. However, only 19% of all ovarian cancers are found at this early stage. Surgery for ovarian cancer includes hysterectomy, bilateral salpingectomy, bilateral oophorectomy and omentectomy. Debulking is performed in women in whom the cancer has spread widely throughout their abdomen. Intraperitoneal (IP) chemotherapy using a combination therapy using a platinum compound, such as cisplatin or carboplatin, and a taxane, such as paclitaxel or docetaxel, is the standard approach. Tumour recurrence is sometimes treated with additional cycles of a platinum compound and/or a taxane. In other cases, recurrence is treated with other drugs, such as topotecan, anthracyclines such as doxorubicin (Adriamycin) and liposomal doxorubicin (Doxil), gemcitabine, cyclophosphamide, vinorelbine (Navelbine), hexamethylmelamine, ifosfamide, and etoposide. Resistance to currently-available chemotherapeutic agents is a major problem. Although complete clinical response is achieved in 75% of patients after initial treatment, most will develop recurrent disease and require re-treatment. External beam radiation therapy can also sometimes be used. For stage IA 92.7%, for stage IB 85.4%, for stage IC 84.7%, for stage HA 78.6%, for stage JIB 72.4%, for stage IIC 64.4%, for stage IIIA 50.8%, for stage IIIB 42.4%, for stage IIIC 31.5% and for stage IV 17.5%.

Pancreatic Cancer—Pancreatic cancer is a very difficult cancer to detect and the prognosis for patients is usually very poor. The number of new cases and deaths per year is almost equal. Global incidence of pancreatic cancer is approximately 230,000 cases (about 2% of all cancer cases), with about 225,000 deaths (3.4% of cancer deaths) per year. It is much more prevalent in the developed world. In the USA, there are about 34,000 new cases per year, with about 32,000 deaths. It has been estimated that approximately US$1.5 billion is spent in the USA each year on treating pancreatic cancer. Pancreatic cancer is very difficult to detect and very few pancreatic cancers are found early. Patients usually have no symptoms until the cancer has spread to other organs. There are currently no blood tests or easily available screening tests that can accurately detect early cancers of the pancreas. An endoscopic ultrasound followed by a biopsy is the best way to diagnose pancreatic cancer. Other detection methods include CT, CT-guided needle biopsy, PET, ultrasonography and MRI. Blood levels of CA 19-9 and carcinoembryonic antigen (CEA) may be elevated but by the time blood levels are high enough to be detected, the cancer is no longer in its early stages. Pancreatic cancer has four stages, stage I to stage IV according to the American Joint Committee on Cancer (AJCC) TNM system. Pancreatic cancer is also divided into resectable, locally advanced (unresectable) and metastatic cancer. For patients with advanced cancers, the overall survival rate is <1% at 5 years with most patients dying within 1 year. Surgery is the only method of curing pancreatic cancer. About 10% of pancreatic cancers are contained entirely within the pancreas at the time of diagnosis and attempts to remove the entire cancer by surgery may be successful in some of these patients. The 5-year survival for those undergoing surgery with the intent of completely removing the cancer is about 20%. Potentially curative surgery, usually by pancreaticoduodenectomy (Whipple procedure), is used when it may be possible to remove all of the cancer. Palliative surgery may be performed if the tumour is too widespread to be completely removed. Removing only part of the cancer does not allow patients to live longer. Pancreatic cancer surgery is difficult to perform with a high likelihood of complications. External beam radiation therapy combined with chemotherapy can be given before or after surgery and can also be given to patients whose tumours are too widespread to be removed by surgery. The main chemotherapeutic agents which are used are gemcitabine and 5-fluorouracil. Targeted therapy using drugs such as erlotinib and cetuximab may be of benefit to patients with advanced pancreatic cancer.

Skin Cancer—Cancer of the skin is the most common of all cancers, probably accounting for more than 50% of all cancers. Melanoma accounts for about 4% of skin cancer cases but causes a large majority of skin cancer deaths. Half of all melanomas are found in people under age 57. About 1 of every 30,000 girls aged 15 to 19 will develop melanoma. For boys of this age, the rate is about 1 of every 15,000. In the USA, about 62,000 new melanomas are diagnosed each year, with around 8,000 deaths. The number of new melanomas diagnosed in the United States is increasing. Among white men and women in the United States, incidence rates for melanoma increased sharply at about 6% per year from 1973 until the early 1980s. Since 1981, however, the rate of increase slowed to little less than 3% per year. Since 1973, the mortality rate for melanoma has increased by 50%. More recently, the death rate from melanoma has leveled off for men and dropped slightly in women. The risk of melanoma is about 20 times higher for whites than for African Americans. Excisional biopsy is the preferred diagnostic method but other types of skin biopsy can also be used including incisional biopsy, shave biopsy and punch biopsy. Metastatic melanoma may not be found until long after the original melanoma was removed from the skin. Metastatic melanoma can be diagnosed using a number of methods including fine needle aspiration biopsy, surgical lymph node biopsy and sentinel lymph node mapping and biopsy. Imaging tests such as a chest x-ray, computed tomography (CT), magnetic resonance imaging (MRI), positron emission tomography (PET) and nuclear bone scans can also be used. Skin cancer is staged using the American Joint Committee on Cancer (AJCC) TNM system—Stage 0-Stage IV. The thickness of a melanoma is measured using the Breslow measurement. Thin melanomas can be completely cured by excision. If the melanoma is on a finger or toe, treatment may involve amputation of the digit. If the melanoma has spread to the lymph nodes, lymph node dissection may be required. No current treatment is usually able to cure stage IV melanoma. Although chemotherapy is usually not as effective in melanoma as in some other types of cancer, it may relieve symptoms or extend survival of some patients with stage IV melanoma. Chemotherapy drugs often used to treat melanoma include dacarbazine, carmustine, cisplatin, vinblastine and temozolomide. Recent studies have found that biochemotherapy, combining several chemotherapy drugs with 1 or more immunotherapy drugs may be more effective than a single chemotherapy drug alone. Immunotherapy drugs include interferon-alpha and/or interleukin-2. Both drugs can help shrink metastatic (stage III and IV) melanomas in about 10% to 20% of patients. Interferon-alpha2b given to patients with stage III melanoma following surgery may delay the recurrence of melanoma. Isolated limb perfusion, using Melphalan, is an experimental type of chemotherapy sometimes used to treat metastatic melanomas confined to the arms or legs. Radiation therapy may be used to treat recurrent melanoma and is used as palliation of metastases to the bone and brain. A person who has already had melanoma has an increased risk of developing melanoma again. In one study, about 11% of people with melanoma developed a second one within 5 years. And those that developed a second melanoma had a 30% chance of developing a third one in 5 years.

Thyroid Cancer—The two most common types of thyroid cancer are papillary carcinoma which accounts for 80% of thyroid cancers and follicular carcinoma which accounts for 10% of thyroid cancers. These are differentiated thyroid cancers which develop from the thyroid follicular cells. Papillary carcinomas grow very slowly; they often spread to lymph nodes in the neck but most of the time, this can be successfully treated and is rarely fatal. Follicular carcinomas usually don't spread to the lymph nodes but can spread to other parts of the body, such as the lungs or bones. The prognosis is not as good as for papillary carcinoma but it still very good in most cases. Other types of thyroid cancer include Hurthle cell carcinoma, medullary thyroid carcinoma and anaplastic carcinoma all of which are less common but harder to treat and have a worse prognosis than papillary carcinoma and follicular carcinoma. There are around 37,000 new cases of thyroid cancer each year in the United States with about 1,600 deaths. The 5-year survival rate is very good at about 97%. Thyroid cancer mainly affects younger people with around 66% of cases found in people between the ages of 20 and 55. Thyroid cancer is diagnosed by fine needle aspiration biopsy. Imaging tests such as a chest x-ray to see if the cancer has spread to the lungs, an ultrasound, a computed tomography (CT) scan, a magnetic resonance imaging (MRI) scan or a radioiodine scan may also be performed. Blood levels of thyroid-stimulating hormone (TSH) may be checked to determine the activity of the thyroid gland. For medullary thyroid carcinoma, levels of calcitonic and carcinoembryonic antigen (CEA) are often high so this can be measured to aid in diagnosis. Thyroid cancer has four stages, stage I to stage IV according to the American Joint Committee on Cancer (AJCC) TNM system. Unlike most other cancers, thyroid cancers are grouped into stages in a way that takes into account both the subtype of cancer and the patient's age. For papillary or follicular thyroid carcinoma, all people under the age of 45 are either stage I or stage II. Patients 45 years and older can be stage I-IV. Stage grouping for medullary thyroid carcinoma in people of any age is the same as for papillary or follicular carcinoma in people older than age 45. All anaplastic thyroid cancers are considered stage IV, reflecting the poor prognosis of this type of cancer. Surgery is the main treatment for thyroid cancer and is used in almost every case, except some anaplastic thyroid cancers. Lobectomy can be used for small differentiated thyroid cancers but thyroidectomy is the most common surgery. Lymph node removal is performed when the cancer has spread outside the thyroid gland. Patients who have undergone total thyroidectomy will need to take daily thyroid hormone replacement pills. Radioactive iodine can be used to destroy any thyroid tissue not removed by surgery or to treat thyroid cancer that has spread to lymph nodes and other parts of the body. Radioactive iodine therapy is not used to treat anaplastic and medullary thyroid carcinomas because these types of cancer do not take up iodine. External beam radiation therapy can be used in these cases. Relative 5-year survival rates by stage for papillary thyroid cancer are: Stage I: 100%; Stage II: 100%; Stage III: 96%; Stage IV: 45%; for follicular thyroid cancer: Stage I: 100%; Stage II: 100%; Stage III: 79%; Stage IV: 47%; for medullary thyroid cancer: Stage I: 100%; Stage II: 97%; Stage III: 78%; Stage IV: 24% and for anaplastic thyroid cancer all are stage IV and the relative 5-year survival rate is around 9%.

Preferred features of each aspect of the invention are as for each of the other aspects mutatis mutandis. The prior art documents mentioned herein are incorporated to the fullest extent permitted by law.

The invention is illustrated by the following non-limiting examples.

### Example 1

### Identification of ROR1 Expressed in Pancreatic Cancer or Breast Cancer Cell Lines Using 1D Gel Electrophoresis

Using the following Reference Protocol, membrane proteins extracted from pancreatic cancer or breast cancer cells were separated by 1D gel and analysed.

### 1.1 Materials and Methods

1.1.1 Plasma Membrane Fractionation

The cells recovered from the pancreatic cancer or breast cancer cell lines were lysed and submitted to centrifugation at 1000 G. The supernatant was taken, and it was subsequently centrifuged at 3000 G. Once again, the supernatant was taken, and it was then centrifuged at 100 000 G. The resulting pellet was recovered and put on 15-60% sucrose gradient. A Western blot was used to identify sub cellular markers, and the plasma membrane fractions were pooled. The pooled solution was either run directly on 1D gels (see section 1.1.4 below), or further fractionated into heparin binding and nucleotide binding fractions as described below.

1.1.2 Plasma Membrane Heparin-Binding Fraction

The pooled solution from 1.1.1 above was applied to a Heparin column, eluted from column and run on 1D gels (see section 1.1.4 below).

1.1.3 Plasma Nucleotide-Binding Fraction

The pooled solution from 1.1.1 above was applied to a Cibacrom Blue 3GA column, eluted from column and run on 1D gels (see section 1.1.4 below).

1.1.4 ID Gel technology

Protein or membrane pellets were solubilised in 1D sample buffer (1-2 μg/μl). The sample buffer and protein mixture was then heated to 95° C. for 3 min. A 9-16% acrylamide gradient gel was cast with a stacking gel and a stacking comb according to the procedure described in Ausubel F. M. et al., eds., 1989, Current Protocols in Molecular Biology, Vol. II, Green Publishing Associates, Inc., and John Wiley & Sons, Inc., New York, section 10.2, incorporated herein by reference in its entirety.

30-50 micrograms of the protein mixtures obtained from detergent and the molecular weight standards (66, 45, 31, 21,14 kDa) were added to the stacking gel wells using a 10 microlitre pipette tip and the samples run at 40 mA for 5 hr. The plates were then prised open, the gel placed in a tray of fixer (10% acetic acid, 40% ethanol, 50% water) and shaken overnight. Following this, the gel was primed by 30 minutes shaking in a primer solution (7.5% acetic acid (75 ml), 0.05% SDS (5 ml of 10%)). The gel was then incubated with a fluorescent dye (7.5% acetic acid, 0.06% OBT in-house dye (6000) with shaking for 3 hrs. Sypro Red (Molecular Probes, Inc., Eugene, Oreg.) is a suitable dye for this purpose. A preferred fluorescent dye is disclosed in U.S. application Ser. No. 09/412,168, filed on Oct. 5, 1999, which is incorporated herein by reference in its entirety. A computer-readable output was produced by imaging the fluorescently stained gels with an Apollo 3 scanner (Oxford BioTherapeutics, Oxford, UK). This scanner is developed from the scanner described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. The latest embodiment of this instrument includes the following improvements: The gel is transported through the scanner on a precision lead-screw drive system. This is preferable to laying the glass plate on the belt-driven system that is defined in the Basiji thesis as it provides a reproducible means of accurately transporting the gel past the imaging optics.

The gel is secured into the scanner against three alignment stops that rigidly hold the glass plate in a known position. By doing this in conjunction with the above precision transport system and the fact that the gel is bound to the glass plate, the absolute position of the gel can be predicted and recorded. This ensures that accurate co-ordinates of each feature on the gel can be communicated to the cutting robot for excision. This cutting robot has an identical mounting arrangement for the glass plate to preserve the positional accuracy. The carrier that holds the gel in place has integral fluorescent markers (Designated M1, M2, M3) that are used to correct the image geometry and are a quality control feature to confirm that the scanning has been performed correctly. The optical components of the system have been inverted. The laser, mirror, waveguide and other optical components are now above the glass plate being scanned. The embodiment of the Basiji thesis has these underneath. The glass plate is therefore mounted onto the scanner gel side down, so that the optical path remains through the glass plate. By doing this, any particles of gel that may break away from the glass plate will fall onto the base of the instrument rather than into the optics. In scanning the gels, they were removed from the stain, rinsed with water and allowed to air dry briefly and imaged on the Apollo 3. After imaging, the gels were sealed in polyethylene bags containing a small volume of staining solution, and then stored at 4° C.

Apparent molecular weights were calculated by interpolation from a set of known molecular weight markers run alongside the samples.

1.1.5 Recovery and Analysis of Selected Proteins

Proteins were robotically excised from the gels by the process described in U.S. Pat. No. 6,064,754, Sections 5.4 and 5.6, 5.7, 5.8 (incorporated herein by reference), as is applicable to 1D-electrophoresis, with modification to the robotic cutter as follows: the cutter begins at the top of the lane, and cuts a gel disc 1.7 mm in diameter from the left edge of the lane. The cutter then moves 2 mm to the right, and 0.7 mm down and cuts a further disc. This is then repeated. The cutter then moves back to a position directly underneath the first gel cut, but offset by 2.2 mm downwards, and the pattern of three diagonal cuts are repeated. This is continued for the whole length of the gel. NOTE: If the lane is observed to broaden significantly then a correction can be made also sideways i.e. instead of returning to a position directly underneath a previous gel cut, the cut can be offset slightly to the left (on the left of the lane) and/or the right (on the right of the lane). The proteins contained within the gel fragments were processed to generate tryptic peptides; partial amino acid sequences of these peptides were determined by mass spectroscopy as described in WO98/53323 and U.S. application Ser. No. 09/094,996, filed Jun. 15, 1998.

Proteins were processed to generate tryptic digest peptides. Tryptic peptides were analyzed by mass spectrometry using a PerSeptive Biosystems Voyager-DE™ STR Matrix-Assisted Laser Desorption Ionization Time-of-Flight (MALDI-TOF) mass spectrometer, and selected tryptic peptides were analyzed by tandem mass spectrometry (MS/MS) using a Micromass Quadrupole Time-of-Flight (Q-TOF) mass spectrometer (Micromass, Altrincham, U.K.) equipped with a Nanoflow™ electrospray Z-spray source. For partial amino acid sequencing and identification of ROR1, uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database, and a mass increment for all Cys residues to account for carbamidomethylation. The database searched was a database constructed of protein entries in the non-redundant database held by the National Centre for Biotechnology Information (NCBI) which is accessible at www.ncbi.nlm.nih.gov. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662).

1.1.6 Discrimination of Pancreatic Cancer Associated Proteins

The process to identify ROR1 uses the peptide sequences obtained experimentally by mass spectrometry described above of naturally occurring human proteins to identify and organize coding exons in the published human genome sequence.

These experimentally determined sequences were compared with the OGAP® database which was compiled by processing and integration of peptide masses, peptide signatures, ESTs and Public Domain Genomic Sequence Data as described in International Patent Application WO2009/087462. The process was used to generate approximately 1 million peptide sequences to identify protein-coding genes and their exons resulted in the identification of protein sequences for 18083 genes across 67 different tissues and 57 diseases including 506 genes in Bladder cancer, 4,713 genes in Breast cancer, 766 genes in Burkitt's lymphoma, 1,371 genes in Cervical cancer, 949 genes in Colorectal cancer, 1,782 genes in Hepatocellular carcinoma, 2,424 genes in CLL, 978 genes in Lung cancer, 1,764 genes in Melanoma, 1,033 genes in Ovarian Cancer, 2,961 genes in Pancreatic cancer and 3,307 genes in Prostate cancer.

### 1.2 Results

These experiments identified ROR1, as further described herein. The full-length ROR1 was detected in the plasma membrane of pancreatic cancer and breast cancer samples and was not detected in the cytosol (FIGS. 1a and 1b). Comparison of the experimentally determined sequences with sequences in the OGAP® database, indicated that ROR1 showed a high degree of specificity to pancreatic cancer (Table 1a) and breast cancer (Table 1b) indicative of the prognostic and diagnostic nature of this protein.

TABLE 1a
1D-Gel Pancreatic Cancer

| Sample | Experiment | |
|---|---|---|
| No. | No. | Peptides identified |
| 1 | 1 | SEQ ID No: 2 - EVVSSTGVLFVK |

TABLE 1b
1D-Gel Breast Cancer

| Sample | Experiment | |
|---|---|---|
| No. | No. | Peptides identified |
| 1 | 1 | SEQ ID No: 3 - |
| | | GTRPPLLALLAALLLAAR |
| 2 | 1 | SEQ ID No: 3 - |
| | | GTRPPLLALLAALLLAAR |

### Example 2

### Identification of Membrane Proteins Expressed in Colorectal Cancer, Kidney Cancer or Lung Cancer Tissue Samples Using Isotope Tagging for Absolute and Relative Quantitation (iTRAQ)

Using the following Reference Protocol, membrane proteins extracted from colorectal cancer, kidney cancer or lung cancer tissues and normal adjacent colorectal cancer, kidney cancer or lung cancer tissue samples were digested, labeled with Isotope Tagging for Absolute & Relative Quantitation reagents (iTRAQ; Applied Biosystems, Foster City, Calif., USA) and resulting peptides sequenced by tandem mass spectrometry.

### 2.1 Materials and Methods

### 2.1.1 Plasma Membrane Fractionation

The cells recovered from a colorectal cancer, kidney cancer or lung cancer or a normal adjacent tissues from colorectal, kidney or lung were lysed and submitted to centrifugation at 1000 G. The supernatant was taken, and it was subsequently centrifuged at 3000 G. Once again, the supernatant was taken, and it was then centrifuged at 100 000 G. The resulting pellet was recovered and put on 15-60% sucrose gradient. A Western blot was used to identify sub cellular markers, and the Plasma Membrane fractions were pooled. The pooled solution was then analyzed directly by iTRAQ (see section 2.1.2 below).

2.1.2 iTRAQ methodology

Membrane protein pellets from colorectal cancer, kidney cancer or lung cancer tissues and normal adjacent tissues from colorectal, kidney or lung were solubilized in sample buffer (2-4 μg/μl in 0.5% SDS) by the addition of buffer and then heating to 95° C. for 3 min. To a volume of each protein solution equating to 50 μg, 150 μl of 0.5M triethylammonium bicarbonate (TEAB) solution was added. To each sample, 3 μl of 50 mM tris-(2-carboxyethyl)phosphine was added and the mixture was incubated at 60° C. for 1 hr. 1 ml of cysteine blocking reagent, 200 mM methyl methanethiosulphonate (MMTS) in isopropanol, was then added. After incubation at room temperature for 10 min, 15 μl of 1 μg/μl trypsin was added to each sample followed by incubation at 37° C. overnight. The digested samples were dried under a vacuum and re-constituted with 30 μl of 0.5M TEAB solution. 70 μl ethanol was added to each of the four iTRAQ reagents (114/115/116/117) and one reagent added to each of the four samples analyzed (each sample comprising two cancer tissue samples and two corresponding normal adjacent tissue samples) and left at room temperature for 1 hr. The specific reagent added to each sample was recorded. The four labeled samples were combined & vortexed. The combined samples was reduced to dryness under a vacuum and de-salted by loading onto a C18 spin column, washing with aqueous solvent and then eluting with 70% acetonitrile. The sample fraction was again reduced to dryness and then re-dissolved in 40 μl of solvent A (97.9 water, 2% acetonitrile, 0.1% formic acid) prior to ion exchange fractionation.

### 2.1.3 Fractionation and Analysis of Labeled Peptides

The sample was fractionated by strong cation exchange chromatography using an Agilent 1200 chromatograph (Agilent, Santa Clara, Calif., USA). Samples were eluted off an Agilent Zorbax Bio-SCXII column (3.5 μm; 50×0 8 mm) using a 20 μl/min gradient of 0-100 mM sodium acetate over 20 min and then to 1M over 10 min. 1 min fractions were collected over the 30 min run.

Each fraction was analyzed by liquid chromatography/mass spectrometry using an Agilent 1200 chromatograph fitted with a Zorbax 300SB-C18 (150 mm×75 mm) and an Agilent 6510 quadrupole-time-of-flight instrument (Agilent, Santa Clara, Calif., USA). Peptides were eluted with a 300n1/min gradient increasing from 15% to 45% acetonitrile in 60 min Data was acquired in auto MS/MS mode such that up to 3 precursor ions above the intensity threshold were selected and product ion spectra accumulated to facilitate the sequencing of the labeled peptides. Raw was processed to create peak lists using Spectrum Mill software (Agilent, Santa Clara, Calif., USA).

### 2.1.4 Amino Acid Sequence Analysis of Labeled Peptides

For partial amino acid sequencing and identification of tyrosine-protein kinase transmembrane receptor ROR1 (ROR1), uninterpreted tandem mass spectra of tryptic peptides were searched using the SEQUEST search program (Eng et al., 1994, J. Am. Soc. Mass Spectrom., 5:976-989). Criteria for database identification included: the cleavage specificity of trypsin; the detection of a suite of a, b and y ions in peptides returned from the database, and a mass increment for all cysteine residues to account for modification with methyl methanethiosulphonate and the addition of iTRAQ labels to free amines (N-terminus & lysine). The data was searched through IPI Human v3.23 (www.ebi.ac.uk/IPI/IPIhuman.html).

### 2.1.5 Discrimination of Colorectal Cancer, Kidney Cancer or Lung Cancer Tissues Associated Proteins

The process to identify ROR1 used the peptide sequences obtained experimentally by mass spectrometry, as described above, of naturally occurring human proteins to identify and organize coding exons in the published human genome sequence. These experimentally determined sequences were compared with the OGAP® database which was compiled by processing and integration of peptide masses, peptide signatures, ESTs and Public Domain Genomic Sequence Data as described in International Patent Publication WO2009/087462.

### 2.2 Results

The experiment identified ROR1, as further described herein (Table 2a, 2b and 2c). The full-length ROR1 was detected in the plasma membrane of non-small cell lung cancer samples (FIGS. 2a, 2b and 2c). The iTRAQ analysis showed that the level of ROR1 in the cancer samples were higher than in the matched normal adjacent tissue samples.

TABLE 2a
iTRAQ Colorectal Cancer

| Sample | Experiment | |
|---|---|---|
| No. | No. | Peptides identified |
| 1 | 1 | SEQ ID No: 4 - ELPLSAVR |
| 2 | 1 | SEQ ID No: 4 - ELPLSAVR |
| 3 | 1 | SEQ ID No: 5 - SNPMILMR |

TABLE 2b
iTRAQ Kidney Cancer

| Sample | Experiment | |
|---|---|---|
| No. | No. | Peptides identified |
| 1 | 1 | SEQ ID No: 6 - |
| | | SNPMILMRLK |
| 2 | 1 | SEQ ID No: 5 - |
| | | SNPMILMR |
| | | SEQ ID No: 6 - |
| | | SNPMILMRLK |
| 3 | 1 | SEQ ID No: 7 - |
| | | GHLYLPGMDHAQLVAIK |

TABLE 2c
iTRAQ Lung Cancer

| Sample | Experiment | |
|---|---|---|
| No. | No. | Peptides identified |
| 1 | 1 | SEQ ID No: 6 - |
| | | SNPMILMRLK |
| 2 | 1 | SEQ ID No: 6 - |
| | | SNPMILMRLK |
| 3 | 1 | SEQ ID No: 5 - |
| | | SNPMILMR |
| | | SEQ ID No: 7 - |
| | | GHLYLPGMDHAQLVAIK |

### Example 3

### Immunohistochemistry Using Antibody To ROR1

Using the following Reference Protocol, immunohistochemistry was performed on FFPE tumour and normal tissues using a goat polyclonal antibody to ROR1 (R&D Systems Europe, Abingdon, UK).

### 3.1 Materials and Methods

### 3.1.1 Deparaffinisation and Rehydration

Slides were heated for 2 hr at 60° C. in 50 ml Falcons in a water bath with no buffer. Each Falcon had one slide or two slides back-to back with long gel loading tip between them to prevent slides from sticking to each other. Slides were deparaffinised in EZ-DeWax (BioGenex, CA, USA) for 5 min in black slide rack, then rinsed well with the same DeWax solution using 1 ml pipette, then washed with water. Slides were placed in a coplin jar filled with water until the pressure cooker was ready; the water was changed a couple of times.

### 3.1.2 Antigen Retrieval

Water was exchanged for antigen retrieval solution=1× citrate buffer, pH 6 (DAKO). Antigen was retrieved by the pressure cooker method. The slides in the plastic coplin jar in antigen retrieval solution were placed into a pressure cooker which was then heated up to position 6 (the highest setting). 15-20 min into the incubation, the temperature was reduced to position 3 and left at that (when the temperature inside the pressure cooker was 117° C.) for another 20-25 min. Then the hob was switched off and the cooker was placed onto the cold hob and the pressure was released by carefully moving the handle into the position between “open” and “closed”. The whole system was left to release the pressure and to cool down for another 20 min. The lid was opened and samples taken out to rest on the bench. The slides were washed 1×5 min with PBS-3T (0.5 L PBS+3 drops of Tween-20) and the slides were placed in PBS.

### 3.1.3 Staining

After antigen retrieval, slides were mounted in the Shandon Coverplate system. Trapping of air bubbles between the slide and plastic coverplate was prevented by placing the coverplate into the coplin jar filled with PBS and gently sliding the slide with tissue sections into the coverplate. The slide was pulled out of the coplin jar while holding it tightly together with the coverplate. The assembled slide was placed into the rack, letting PBS trapped in the funnel and between the slide and coverplate to run through. Slides were washed with 2×2 ml (or 4×1 ml) PBS-3T and 1×2 ml PBS, waiting until all PBS had gone through the slide and virtually no PBS was left in the funnel

Endogenous peroxide blockade was performed using peroxidase blocking reagent (S2001, DAKO). 1-4 drops of peroxide solution was used per slide and incubated for 5 minutes. The slides were rinsed with water and then once with 2 ml PBS-3T and once with 2 ml PBS; it was important to wait until virtually no liquid was left in the funnel before adding a new portion of wash buffer.

The primary antibody was diluted with an Antibody diluent reagent (DAKO). Optimal dilution was determined to be 1:100. 50-200 μl of diluted primary antibody was applied to each section and/or tissue microarray; taking care to cover the whole tissue. The slide was gently tapped to distribute the antibody evenly over the section or a pipette tip was used over the top of the section. The slide was incubated for 45 min in a moist chamber at room temperature. Slides were washed with 2×2 ml (or 4×1 ml) PBS-3T and then 1×2 ml PBS, waiting until all PBS had gone through the slide and virtually no PBS was left in the funnel. The corresponding donkey anti-goat IgG:HRP (OBT1500P, 1 mg/ml, Serotec) was applied at 1:1000 and incubated for 35 min at room temperature. The slides were washed as above. The DAB substrate was made up in dilution buffer; 2 ml containing 2 drops of substrate was enough for 10 slides. The DAB reagent was applied to the slides by applying a few drops at a time. All of the DAB was distributed between the slides. The slides were incubated for 10 min. The slides were washed 1×2 ml (or 2×1 ml) with PBS-3T and 1×2 ml (or 2×1 ml) with PBS, waiting until all PBS had gone through the slide and virtually no PBS was left in the funnel Hematoxylin (DAKO) was applied; 1 ml was enough for 10 slides and slides were incubated for 1 min at room temperature. The funnels of the Shandon Coverplate system were filled with 2 ml of water and let to run through. When slides were clear of the excess of hematoxylin, the system was disassembled, tissue sections and/or arrays were washed with water from the wash bottle and placed into a black slide rack. Tissues were rehydrated by incubating in EZ-DeWax for 5 min and then in 95% ethanol for 2-5 min. Slides were left to dry on the bench at room temperature and then mounted in mounting media and covered with coverslip.

3.2 Results

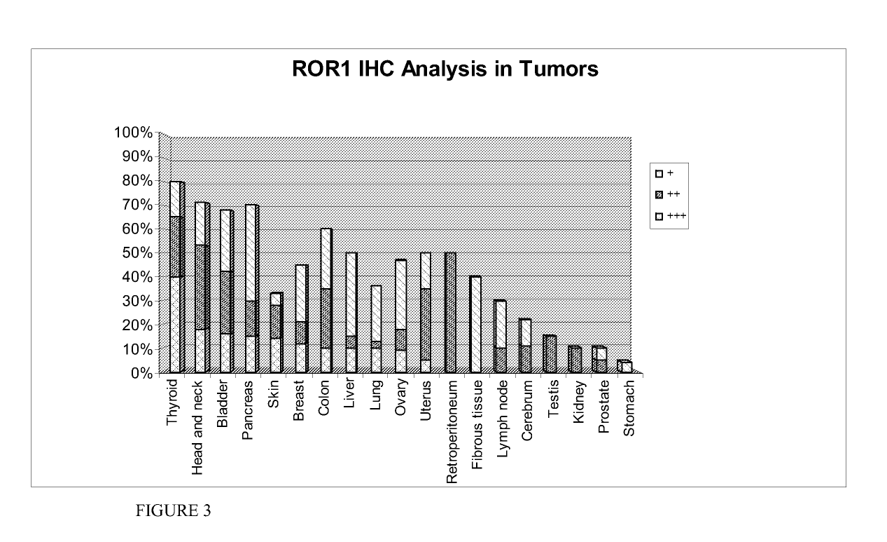

Immunohistochemical analysis revealed specific staining of tumor cells in pancreatic cancer tissue sections. FIG. 3 shows the results of a high density array (Biomax, US) containing 500 tissue cores from the 20 most common types of cancer (20 cases/type) and normal controls (5 cases/type). FIG. 2 indicates the % prevalence and staining at different intensities (+=weak staining; ++=moderate staining; +++=strong staining) for each tumor type. Elevated staining of ROR1 in cancer cells was seen in bladder cancer, breast cancer, colorectal cancer, head and neck cancer, kidney cancer, liver cancer, lung cancer, ovarian cancer, pancreatic cancer, skin cancer and thyroid cancer.

### Example 4

### RNA Profiling of ROR1

### 4.1 Materials and Methods

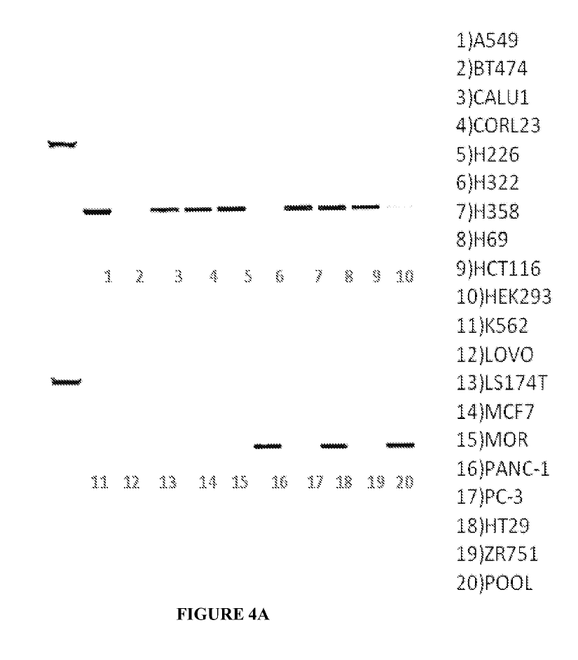

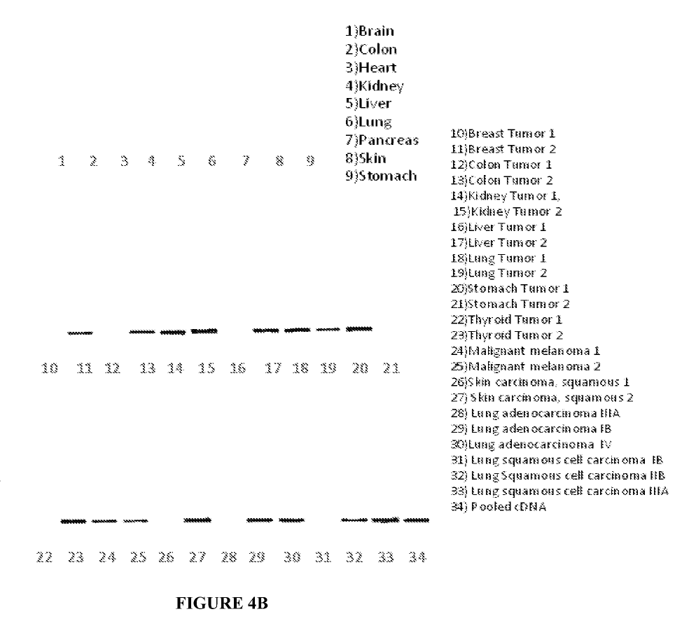

Gene expression data for ROR1 was obtained on 19 cancer cell lines, 24 cancer tissues and 9 normal tissues (FIGS. 4a and 4b). The oligonucleotide primer set was chosen to represent the extracellular domain of ROR1 (SEQ ID No: 8).

### 4.2 Results

The analysis of the 19 cancer cell lines indicated ROR1 expression in wide range of cancer cells (A549, CALU1, H226, H322, H358 H69, HCT116, HEK293, PANC-1 and HT-29) (FIG. 4a). The RT-PCT analysis using cancer tissues also showed ROR1 expression in various cancer tissues (breast cancer, kidney cancer, liver cancer, lung cancer, stomach cancer, thyroid cancer, malignant melanoma, lung adenocarcinoma and lung lung squamous cell carcinoma) (FIG. 4b). The extracellular domain of ROR1 was virtually undetectable in the normal tissues tested (FIG. 4b).

### Example 5

### Construction of a Phage-Display Library

A recombinant protein composed of the extracellular domain of the ROR1 (SEQ ID NO:8) was eurkaryotically synthesized by standard recombinant methods and used as antigen for immunization.

Immunization and mRNA Isolation

A phage display library for identification of the ROR1-binding molecules was constructed as follows. A/J mice (Jackson Laboratories, Bar Harbor, Me.) were immunized intraperitoneally with the recombinant ROR1 antigen (the extracellular domain), using 100 μg protein in Freund's complete adjuvant, on day 0, and with 100 μg antigen on day 28. Test bleeds of mice were obtained through puncture of the retro-orbital sinus. If, by testing the titers, they were deemed high by ELISA using the biotinylated ROR1 antigen immobilized via neutravidin (Reacti-Bind™) NeutrAvidin™-Coated Polystyrene Plates, Pierce, Rockford, Ill.), the mice were boosted with 100 μg of protein on day 70, 71 and 72, with subsequent sacrifice and splenectomy on day 77. If titers of antibody were not deemed satisfactory, mice were boosted with 100 μg antigen on day 56 and a test bleed taken on day 63. If satisfactory titers were obtained, the animals were boosted with 100 μg of antigen on day 98, 99, and 100 and the spleens harvested on day 105.

The spleens were harvested in a laminar flow hood and transferred to a petri dish, trimming off and discarding fat and connective tissue. The spleens were macerated quickly with the plunger from a sterile 5 cc syringe in the presence of 1.0 ml of solution D (25.0 g guanidine thiocyanate (Boehringer Mannheim, Indianapolis, Ind.), 29.3 ml sterile water, 1.76 ml 0.75 M sodium citrate pH 7.0, 2.64 ml 10% sarkosyl (Fisher Scientific, Pittsburgh, Pa.), 0.36 ml 2-mercaptoethanol (Fisher Scientific, Pittsburgh, Pa.). This spleen suspension was pulled through an 18 gauge needle until all cells were lysed and the viscous solution was transferred to a microcentrifuge tube. The petri dish was washed with 100 μl of solution D to recover any remaining spleen. This suspension was then pulled through a 22 gauge needle an additional 5-10 times.

The sample was divided evenly between two microcentrifuge tubes and the following added, in order, with mixing by inversion after each addition: 50 μl 2 M sodium acetate pH 4.0, 0.5 ml water-saturated phenol (Fisher Scientific, Pittsburgh, Pa.), 100 μl chloroform/isoamyl alcohol 49:1 (Fisher Scientific, Pittsburgh, Pa.). The solution was vortexed for 10 sec and incubated on ice for 15 min. Following centrifugation at 14 krpm for 20 min at 2-8° C., the aqueous phase was transferred to a fresh tube. An equal volume of water saturated phenol:chloroform:isoamyl alcohol (50:49:1) was added, and the tube vortexed for ten seconds. After 15 min incubation on ice, the sample was centrifuged for 20 min at 2-8° C., and the aqueous phase transferred to a fresh tube and precipitated with an equal volume of isopropanol at −20° C. for a minimum of 30 min. Following centrifugation at 14 krpm for 20 min at 4° C., the supernatant was aspirated away, the tubes briefly spun and all traces of liquid removed from the RNA pellet.

The RNA pellets were each dissolved in 300 ml of solution D, combined, and precipitated with an equal volume of isopropanol at −20° C. for a minimum of 30 min. The sample was centrifuged 14 krpm for 20 min at 4° C., the supernatant aspirated as before, and the sample rinsed with 100 μl of ice-cold 70% ethanol. The sample was again centrifuged 14 krpm for 20 min at 4° C., the 70% ethanol solution aspirated, and the RNA pellet dried in vacuo. The pellet was resuspended in 100 μl of sterile diethyl pyrocarbonate-treated water. The concentration was determined by A260 using an absorbance of 1.0 for a concentration of 40 μg/ml. The RNAs were stored at −80° C.

Preparation of Complementary DNA (cDNA)

The total RNA purified from mouse spleens as described above was used directly as template for cDNA preparation. RNA (50 μg) was diluted to 100 μL with sterile water, and 10 μL of 130 ng/μL oligo dT12 (synthesized on Applied Biosystems Model 392 DNA synthesizer) was added. The sample was heated for 10 min at 70° C., then cooled on ice. Forty μL 5* first strand buffer was added (Gibco/BRL, Gaithersburg, Md.), along with 20 μL 0.1 M dithiothreitol (Gibco/BRL, Gaithersburg, Md.), 10 μL 20 mM deoxynucleoside triphosphates (dNTP's, Boehringer Mannheim, Indianapolis, Ind.), and 10 μL water on ice. The sample was then incubated at 37° C. for 2 min. Ten μL reverse transcriptase (Superscript™) II, Gibco/BRL, Gaithersburg, Md.) was added and incubation was continued at 37° C. for 1 hr. The cDNA products were used directly for polymerase chain reaction (PCR).

Amplification of Antibody Genes by PCR

To amplify substantially all of the H and L chain genes using PCR, primers were chosen that corresponded to substantially all published sequences. Because the nucleotide sequences of the amino termini of H and L contain considerable diversity, 33 oligonucleotides were synthesized to serve as 5′ primers for the H chains, and 29 oligonucleotides were synthesized to serve as 5′ primers for the kappa L chains as described in U.S. Pat. No. 6,555,310. The constant region nucleotide sequences for each chain required only one 3′ primer for the H chains and one 3′ primer for the kappa L chains.

A 50 μL reaction was performed for each primer pair with 50 μmol of 5′ primer, 50 μmol of 3′ primer, 0.25 μL Taq DNA Polymerase (5 units/μL, Boehringer Mannheim, Indianapolis, Ind.), 3 μL cDNA (prepared as described), 5 μL 2 mM dNTP's, 5 μL 10*Taq DNA polymerase buffer with MgCl2 (Boehringer Mannheim, Indianapolis, Ind.), and H₂O to 50 μL. Amplification was done using a GeneAmp(R) 9600 thermal cycler (Perkin Elmer, Foster City, Calif.) with the following thermocycle program: 94° C. for 1 min; 30 cycles of 94° C. for 20 sec, 55° C. for 30 sec, and 72° C. for 30 sec, 72° C. for 6 min; 4° C.

The dsDNA products of the PCR process were then subjected to asymmetric PCR using only a 3′ primer to generate substantially only the anti-sense strand of the target genes. A 100 μL reaction was done for each dsDNA product with 200 μmol of 3′ primer, 2 μL of ds-DNA product, 0.5 μL Taq DNA Polymerase, 10 μL 2 mM dNTP's, 10 μL 10*Taq DNA polymerase buffer with MgCl₂ (Boehringer Mannheim, Indianapolis, Ind.), and H₂O to 100 μL. The same PCR program as that described above was used to amplify the single-stranded (ss)-DNA.

Purification of Single-Stranded DNA by High Performance Liquid Chromatography and Kinasing

Single-Stranded DNA

The H chain ss-PCR products and the L chain single-stranded PCR products were ethanol precipitated by adding 2.5 volumes ethanol and 0.2 volumes 7.5 M ammonium acetate and incubating at −20° C. for at least 30 min. The DNA was pelleted by centrifuging in an Eppendorf centrifuge at 14 krpm for 10 min at 2-8° C. The supernatant was carefully aspirated, and the tubes were briefly spun a 2nd time. The last drop of supernatant was removed with a pipette. The DNA was dried in vacuo for 10 min on medium heat. The H chain products were pooled in 210 μL water and the L chain products were pooled separately in 210 μL water. The single-stranded DNA was purified by high performance liquid chromatography (HPLC) using a Hewlett Packard 1090 HPLC and a Gen-Pak™) FAX anion exchange column (Millipore Corp., Milford, Mass.). The gradient used to purify the single-stranded DNA is shown in Table 3, and the oven temperature was 60° C. Absorbance was monitored at 260 nm. The single-stranded DNA eluted from the HPLC was collected in 0.5 min fractions. Fractions containing single-stranded DNA were ethanol precipitated, pelleted and dried as described above. The dried DNA pellets were pooled in 200 μL sterile water.

TABLE 3
HPLC gradient for purification of ss-DNA

| Time | | | | Flow |
|---|---|---|---|---|
| (min) | % A | % B | % C | (ml/min) |
| 0 | 70 | 30 | 0 | 0.75 |
| 2 | 40 | 60 | 0 | 0.75 |
| 17 | 15 | 85 | 0 | 0.75 |
| 18 | 0 | 100 | 0 | 0.75 |
| 23 | 0 | 100 | 0 | 0.75 |
| 24 | 0 | 0 | 100 | 0.75 |
| 28 | 0 | 0 | 100 | 0.75 |
| 29 | 0 | 100 | 0 | 0.75 |
| 34 | 0 | 100 | 0 | 0.75 |
| 35 | 70 | 30 | 0 | 0.75 |

Buffer A is 25 mM Tris, 1 mM EDTA, pH 8.0
Buffer B is 25 mM Tris, 1 mM EDTA, 1M NaCl, pH 8.0
Buffer C is 40 mm phosphoric acid

The single-stranded DNA was 5′-phosphorylated in preparation for mutagenesis. Twenty-four μL 10* kinase buffer (United States Biochemical, Cleveland, Ohio), 10.4 μL 10 mM adenosine-5′-triphosphate (Boehringer Mannheim, Indianapolis, Ind.), and 2 μL polynucleotide kinase (30 units/μL, United States Biochemical, Cleveland, Ohio) was added to each sample, and the tubes were incubated at 37° C. for 1 hr. The reactions were stopped by incubating the tubes at 70° C. for 10 min. The DNA was purified with one extraction of Tris equilibrated phenol (pH>8.0, United States Biochemical, Cleveland, Ohio):chloroform:isoamyl alcohol (50:49:1) and one extraction with chloroform:isoamyl alcohol (49:1). After the extractions, the DNA was ethanol precipitated and pelleted as described above. The DNA pellets were dried, then dissolved in 50 μL sterile water. The concentration was determined by measuring the absorbance of an aliquot of the DNA at 260 rim using 33 μg/ml for an absorbance of 1.0. Samples were stored at −20° C.

Preparation of Uracil Templates Used in Generation of Spleen Antibody Phage Libraries

One ml of E. coli CJ236 (BioRAD, Hercules, Calif.) overnight culture was added to 50 ml 2*YT in a 250 ml baffled shake flask. The culture was grown at 37° C. to OD600=0.6, inoculated with 10 μl of a 1/100 dilution of BS45 vector phage stock (described in U.S. Pat. No. 6,555,310) and growth continued for 6 hr. Approximately 40 ml of the culture was centrifuged at 12 krpm for 15 min at 4° C. The supernatant (30 ml) was transferred to a fresh centrifuge tube and incubated at room temperature for 15 min after the addition of 15 μl of 10 mg/ml RNaseA (Boehringer Mannheim, Indianapolis, Ind.). The phages were precipitated by the addition of 7.5 ml of 20% polyethylene glycol 8000 (Fisher Scientific, Pittsburgh, Pa.)/3.5M ammonium acetate (Sigma Chemical Co., St. Louis, Mo.) and incubation on ice for 30 min. The sample was centrifuged at 12 krpm for 15 min at 2-8° C. The supernatant was carefully discarded, and the tube briefly spun to remove all traces of supernatant. The pellet was resuspended in 400 μl of high salt buffer (300 mM NaCl, 100 mM Tris pH 8.0, 1 mM EDTA), and transferred to a 1.5 ml tube.

The phage stock was extracted repeatedly with an equal volume of equilibrated phenol:chloroform:isoamyl alcohol (50:49:1) until no trace of a white interface was visible, and then extracted with an equal volume of chloroform:isoamyl alcohol (49:1). The DNA was precipitated with 2.5 volumes of ethanol and ⅕ volume 7.5 M ammonium acetate and incubated 30 min at −20° C. The DNA was centrifuged at 14 krpm for 10 min at 4° C., the pellet washed once with cold 70% ethanol, and dried in vacuo. The uracil template DNA was dissolved in 30 μl sterile water and the concentration determined by A260 using an absorbance of 1.0 for a concentration of 40 μg/ml. The template was diluted to 250 ng/μL with sterile water, aliquoted and stored at −20° C.

Mutagenesis of Uracil Template with Ss-DNA and Electroporation into E. Coli to Generate Antibody Phage Libraries

Antibody phage display libraries were generated by simultaneously introducing single-stranded heavy and light chain genes onto a phage display vector uracil template. A typical mutagenesis was performed on a 2 μg scale by mixing the following in a 0.2 ml PCR reaction tube: 8 μl of (250 ng/μL) uracil template, 8 μL of 10* annealing buffer (200 mM Tris pH 7.0, 20 mM MgCl₂, 500 mM NaCl), 3.33 μl of kinased single-stranded heavy chain insert (100 ng/μL), 3.1 μl of kinased single-stranded light chain insert (100 ng/μL), and sterile water to 80 μA DNA was annealed in a GeneAmp(R) 9600 thermal cycler using the following thermal profile: 20 sec at 94° C., 85° C. for 60 sec, 85° C. to 55° C. ramp over 30 min, hold at 55° C. for 15 min. The DNA was transferred to ice after the program finished. The extension/ligation was carried out by adding 8 μl of 10* synthesis buffer (5 mM each dNTP, 10 mM ATP, 100 mM Tris pH 7.4, 50 mM MgCl₂, 20 mM DTT), 8 μL T4 DNA ligase (1 U/μL, Boehringer Mannheim, Indianapolis, Ind.), 8 μL diluted T7 DNA polymerase (1 U/μL, New England BioLabs, Beverly, Mass.) and incubating at 37° C. for 30 min. The reaction was stopped with 300 μL of mutagenesis stop buffer (10 mM Tris pH 8.0, 10 mM EDTA). The mutagenesis DNA was extracted once with equilibrated phenol (pH>8):chloroform:isoamyl alcohol (50:49:1), once with chloroform:isoamyl alcohol (49:1), and the DNA was ethanol precipitated at −20° C. for at least 30 min. The DNA was pelleted and the supernatant carefully removed as described above. The sample was briefly spun again and all traces of ethanol removed with a pipetman. The pellet was dried in vacuo. The DNA was resuspended in 4 μL of sterile water.

One μL of mutagenesis DNA (500 ng) was transferred into 40 μl electrocompetent E. coliDH12S (Gibco/BRL, Gaithersburg, Md.) using electroporation. The transformed cells were mixed with approximately 1.0 ml of overnight XL-1 cells which were diluted with 2*YT broth to 60% the original volume. This mixture was then transferred to a 15-ml sterile culture tube and 9 ml of top agar added for plating on a 150-mm LB agar plate. Plates were incubated for 4 hr at 37° C. and then transferred to 20° C. overnight. First round antibody phage were made by eluting phage off these plates in 10 ml of 2*YT, spinning out debris, and taking the supernatant. These samples are the antibody phage display libraries used for selecting antibodies against the ROR1. Efficiency of the electroporations was measured by plating 10 μl of a 10⁻⁴ dilution of suspended cells on LB agar plates, follow by overnight incubation of plates at 37° C. The efficiency was calculated by multiplying the number of plaques on the 10⁻⁴ dilution plate by 106. Library electroporation efficiencies are typically greater than 1*10⁷ phages under these conditions.

Transformation of E. Coli by Electroporation

Electrocompetent E. coli cells were thawed on ice. DNA was mixed with 40 L of these cells by gently pipetting the cells up and down 2-3 times, being careful not to introduce an air bubble. The cells were transferred to a Gene Pulser cuvette (0.2 cm gap, BioRAD, Hercules, Calif.) that had been cooled on ice, again being careful not to introduce an air bubble in the transfer. The cuvette was placed in the E. coli Pulser (BioRAD, Hercules, Calif.) and electroporated with the voltage set at 1.88 kV according to the manufacturer's recommendation. The transformed sample was immediately resuspended in 1 ml of 2*YT broth or 1 ml of a mixture of 400 μl 2*YT/600 ml overnight XL-1 cells and processed as procedures dictated.

Plating M13 Phage or Cells Transformed with Antibody Phage-Display Vector Mutagenesis Reaction

Phage samples were added to 200 μL of an overnight culture of E. coli XL1-Blue when plating on 100 mm LB agar plates or to 600 μL of overnight cells when plating on 150 mm plates in sterile 15 ml culture tubes. After adding LB top agar (3 ml for 100 mm plates or 9 ml for 150 mm plates, top agar stored at 55° C. (see, Appendix A1, Sambrook et al., supra.), the mixture was evenly distributed on an LB agar plate that had been pre-warmed (37° C.-55° C.) to remove any excess moisture on the agar surface.

The plates were cooled at room temperature until the top agar solidified. The plates were inverted and incubated at 37° C. as indicated.

Preparation of Biotinylated Tyrosine-Protein Kinase Transmembrane Receptor ROR1 and

Biotinylated Antibodies

The concentrated recombinant ROR1 antigen (full length extracellular domain) was extensively dialyzed into BBS (20 mM borate, 150 mM NaCl, 0.1% NaN₃, pH 8.0). After dialysis, 1 mg of the ROR1 (1 mg/ml in BBS) was reacted with a 15 fold molar excess of biotin-XX-NHS ester (Molecular Probes, Eugene, Oreg., stock solution at 40 mM in DMSO). The reaction was incubated at room temperature for 90 min and then quenched with taurine (Sigma Chemical Co., St. Louis, Mo.) at a final concentration of 20 mM. The biotinylation reaction mixture was then dialyzed against BBS at 2-8° C. After dialysis, the biotinylated ROR1 was diluted in panning buffer (40 mM Tris, 150 mM NaCl, 20 mg/ml BSA, 0.1% Tween 20, pH 7.5), aliquoted, and stored at −80° C. until needed.

Antibodies were reacted with 3-(N-maleimidylpropionyl)-biocytin (Molecular Probes, Eugene, Oreg.) using a free cysteine located at the carboxy terminus of the heavy chain. Antibodies were reduced by adding DTT to a final concentration of 1 mM for 30 min at room temperature. Reduced antibody was passed through a Sephadex G50 desalting column equilibrated in 50 mM potassium phosphate, 10 mM boric acid, 150 mM NaCl, pH 7.0. 3-(N-maleimidylpropionyl)-biocytin was added to a final concentration of 1 mM and the reaction allowed to proceed at room temperature for 60 min. Samples were then dialyzed extensively against BBS and stored at 2-8° C.

Preparation of Avidin Magnetic Latex

The magnetic latex (Estapor, 10% solids, Bangs Laboratories, Fishers, Ind.) was thoroughly resuspended and 2 ml aliquoted into a 15 ml conical tube. The magnetic latex was suspended in 12 ml distilled water and separated from the solution for 10 min using a magnet (PerSeptive Biosystems, Framingham, Mass.). While maintaining the separation of the magnetic latex with the magnet, the liquid was carefully removed using a 10 ml sterile pipette. This washing process was repeated an additional three times. After the final wash, the latex was resuspended in 2 ml of distilled water. In a separate 50 ml conical tube, 10 mg of avidin-HS (NeutrAvidin, Pierce, Rockford, Ill.) was dissolved in 18 ml of 40 mM Tris, 0.15 M sodium chloride, pH 7.5 (TBS). While vortexing, the 2 ml of washed magnetic latex was added to the diluted avidin-HS and the mixture mixed an additional 30 sec. This mixture was incubated at 45° C. for 2 hr, shaking every 30 min. The avidin magnetic latex was separated from the solution using a magnet and washed three times with 20 ml BBS as described above. After the final wash, the latex was resuspended in 10 ml BBS and stored at 4° C.

Immediately prior to use, the avidin magnetic latex was equilibrated in panning buffer (40 mM Tris, 150 mM NaCl, 20 mg/ml BSA, 0.1% Tween 20, pH 7.5). The avidin magnetic latex needed for a panning experiment (200 μl/sample) was added to a sterile 15 ml centrifuge tube and brought to 10 ml with panning buffer. The tube was placed on the magnet for 10 min to separate the latex. The solution was carefully removed with a 10 ml sterile pipette as described above. The magnetic latex was resuspended in 10 ml of panning buffer to begin the second wash. The magnetic latex was washed a total of 3 times with panning buffer. After the final wash, the latex was resuspended in panning buffer to the starting volume.

### Example 6

### Selection of Recombinant Polyclonal Antibodies To ROR1 Antigen

Binding reagents that specifically bind to the ROR1 were selected from the phage display libraries created from hyperimmunized mice as described in Example 5.

### Panning

First round antibody phage were prepared as described in Example 5 using BS45 uracil template. Electroporations of mutagenesis DNA were performed yielding phage samples derived from different immunized mice. To create more diversity in the recombinant polyclonal library, each phage sample was panned separately.

Before the first round of functional panning with the biotinylated ROR1 antigen, antibody phage libraries were selected for phage displaying both heavy and light chains on their surface by panning with 7F11-magnetic latex (as described in Examples 21 and 22 of U.S. Pat. No. 6,555,310). Functional panning of these enriched libraries was performed in principle as described in Example 16 of U.S. Pat. No. 6,555,310. Specifically, 10 μL of 1*10⁻⁶ M biotinylated ROR1 antigen was added to the phage samples (approximately 1*10⁻⁸ M final concentration of the ROR1), and the mixture allowed to come to equilibrium overnight at 2-8° C.

After reaching equilibrium, samples were panned with avidin magnetic latex to capture antibody phage bound to the ROR1. Equilibrated avidin magnetic latex (Example 5), 200 μL latex per sample, was incubated with the phage for 10 min at room temperature. After 10 min, approximately 9 ml of panning buffer was added to each phage sample, and the magnetic latex separated from the solution using a magnet. After a ten minute separation, unbound phage was carefully removed using a 10 ml sterile pipette. The magnetic latex was then resuspended in 10 ml of panning buffer to begin the second wash. The latex was washed a total of three times as described above. For each wash, the tubes were in contact with the magnet for 10 min to separate unbound phage from the magnetic latex. After the third wash, the magnetic latex was resuspended in 1 ml of panning buffer and transferred to a 1.5 mL tube. The entire volume of magnetic latex for each sample was then collected and resuspended in 200 μl 2*YT and plated on 150 mm LB plates as described in Example 1 to amplify bound phage. Plates were incubated at 37° C. for 4 hr, then overnight at 20° C.

The 150 mm plates used to amplify bound phage were used to generate the next round of antibody phage. After the overnight incubation, second round antibody phage were eluted from the 150 mm plates by pipetting 10 mL of 2*YT media onto the lawn and gently shaking the plate at room temperature for 20 min. The phage samples were then transferred to 15 ml disposable sterile centrifuge tubes with a plug seal cap, and the debris from the LB plate pelleted by centrifuging the tubes for 15 min at 3500 rpm. The supernatant containing the second round antibody phage was then transferred to a new tube.

A second round of functional panning was set up by diluting 100 μL of each phage stock into 900 μL of panning buffer in 15 ml disposable sterile centrifuge tubes. The biotinylated ROR1 antigen was then added to each sample as described for the first round of panning, and the phage samples incubated for 1 hr at room temperature. The phage samples were then panned with avidin magnetic latex as described above. The progress of panning was monitored at this point by plating aliquots of each latex sample on 100 mm LB agar plates to determine the percentage of kappa positives. The majority of latex from each panning (99%) was plated on 150 mm LB agar plates to amplify the phage bound to the latex. The 100 mm LB agar plates were incubated at 37° C. for 6-7 hr, after which the plates were transferred to room temperature and nitrocellulose filters (pore size 0.45 mm, BA85 Protran, Schleicher and Schuell, Keene, N.H.) were overlaid onto the plaques.

Plates with nitrocellulose filters were incubated overnight at room temperature and then developed with a goat anti-mouse kappa alkaline phosphatase conjugate to determine the percentage of kappa positives as described below. Phage samples with lower percentages (<70%) of kappa positives in the population were subjected to a round of panning with 7F11-magnetic latex before performing a third functional round of panning overnight at 2-8° C. using the biotinylated ROR1 antigen at approximately 2*10⁻⁹ M. This round of panning was also monitored for kappa positives. Individual phage samples that had kappa positive percentages greater than 80% were pooled and subjected to a final round of panning overnight at 2-8° C. at 5*10⁻⁹ M. The ROR1 antibody genes contained within the eluted phage from this fourth round of functional panning were subcloned into the expression vector, pBRncoH3.

The subcloning process was done generally as described in Example 18 of U.S. Pat. No. 6,555,310. After subcloning, the expression vector was electroporated into DH10B cells and the mixture grown overnight in 2*YT containing 1% glycerol and 10 μg/ml tetracycline. After a second round of growth and selection in tetracycline, aliquots of cells were frozen at −80° C. as the source for the ROR1 polyclonal antibody production. Monoclonal antibodies were selected from these polyclonal mixtures by plating a sample of the mixture on LB agar plates containing 10 μg/ml tetracycline and screening for antibodies that recognized the ROR1.

Expression and Purification of Recombinant Antibodies Against Tyrosine-Protein Kinase

Transmembrane Receptor ROR1

A shake flask inoculum was generated overnight from a −70° C. cell bank in an Innova 4330 incubator shaker (New Brunswick Scientific, Edison, N.J.) set at 37° C., 300 rpm. The inoculum was used to seed a 20 L fermentor (Applikon, Foster City, Calif.) containing defined culture medium [Pack et al. (1993) BioTechnology 11: 1271-1277] supplemented with 3 g/L L-leucine, 3 g/L L-isoleucine, 12 g/L casein digest (Difco, Detroit, Mich.), 12.5 g/L glycerol and 10 μg/ml tetracycline. The temperature, pH and dissolved oxygen in the fermentor were controlled at 26° C., 6.0-6.8 and 25% saturation, respectively. Foam was controlled by addition of polypropylene glycol (Dow, Midland, Mich.). Glycerol was added to the fermentor in a fed-batch mode. Fab expression was induced by addition of L(+)-arabinose (Sigma, St. Louis, Mo.) to 2 g/L during the late logarithmic growth phase. Cell density was measured by optical density at 600 nm in an UV-1201 spectrophotometer (Shimadzu, Columbia, Md.). Following run termination and adjustment of pH to 6.0, the culture was passed twice through an M-210B-EH Microfluidizer (Microfluidics, Newton, Mass.) at 17,000 psi. The high pressure homogenization of the cells released the Fab into the culture supernatant.

The first step in purification was expanded bed immobilized metal affinity chromatography (EB-IMAC). Streamline™ chelating resin (Pharmacia, Piscataway, N.J.) was charged with 0.1 M NiCl₂ and was then expanded and equilibrated in 50 mM acetate, 200 mM NaCl, 10 mM imidazole, 0.01% NaN₃, pH 6.0 buffer flowing in the upward direction. A stock solution was used to bring the culture homogenate to 10 mM imidazole, following which it was diluted two-fold or higher in equilibration buffer to reduce the wet solids content to less than 5% by weight. It was then loaded onto the Streamline column flowing in the upward direction at a superficial velocity of 300 cm/hr. The cell debris passed through unhindered, but the Fab was captured by means of the high affinity interaction between nickel and the hexahistidine tag on the Fab heavy chain. After washing, the expanded bed was converted to a packed bed and the Fab was eluted with 20 mM borate, 150 mM NaCl, 200 mM imidazole, 0.01% NaN₃, pH 8.0 buffer flowing in the downward direction.

The second step in the purification used ion-exchange chromatography (IEC). Q Sepharose FastFlow resin (Pharmacia, Piscataway, N.J.) was equilibrated in 20 mM borate, 37.5 mM NaCl, 0.01% NaN₃, pH 8.0. The Fab elution pool from the EB-IMAC step was diluted four-fold in 20 mM borate, 0.01% NaN₃, pH 8.0 and loaded onto the IEC column. After washing, the Fab was eluted with a 37.5-200 mM NaCl salt gradient. The elution fractions were evaluated for purity using an Xcell II™ SDS-PAGE system (Novex, San Diego, Calif.) prior to pooling. Finally, the Fab pool was concentrated and diafiltered into 20 mM borate, 150 mM NaCl, 0.01% NaN₃, pH 8.0 buffer for storage. This was achieved in a Sartocon Slice™ system fitted with a 10,000 MWCO cassette (Sartorius, Bohemia, N.Y.). The final purification yields were typically 50%. The concentration of the purified Fab was measured by UV absorbance at 280 nm, assuming an absorbance of 1.6 for a 1 mg/ml solution.

### Example 7

### Specificity of Monoclonal Antibodies to ROR1Determined by Flow Cytometry Analysis

The specificity of antibodies against the ROR1 selected in Example 6 was tested by flow cytometry. To test the ability of the antibodies to bind to the cell surface ROR1 protein, the antibodies were incubated with the ROR1-expressing cells, A549, from human lung adenocarinoma. Cells were washed in FACS buffer (DPBS, 2% FBS), centrifuged and resuspended in 100 μl of the diluted primary ROR1 antibody (also diluted in FACS buffer). The antibody-A549 complex was incubated on ice for 60 min and then washed twice with FACS buffer as described above. The cell-antibody pellet was resuspended in 100 μl of the diluted secondary antibody (also diluted in FACS buffer) and incubated on ice for 60 min on ice. The pellet was washed as before and resuspended in 200 μl FACS buffer. The samples were loaded onto the BD FACScanto II flow sytometer and the data analyzed using the BD FACSdiva software. The binding of ROR1_A3, ROR1_A1, ROR1_A8 and ROR1_A14 to ROR1 expressed on A549, H69 (human small cell lung carcinoma) and HT29 (colon carcinoma) cells were also analysed using flow cytometry.

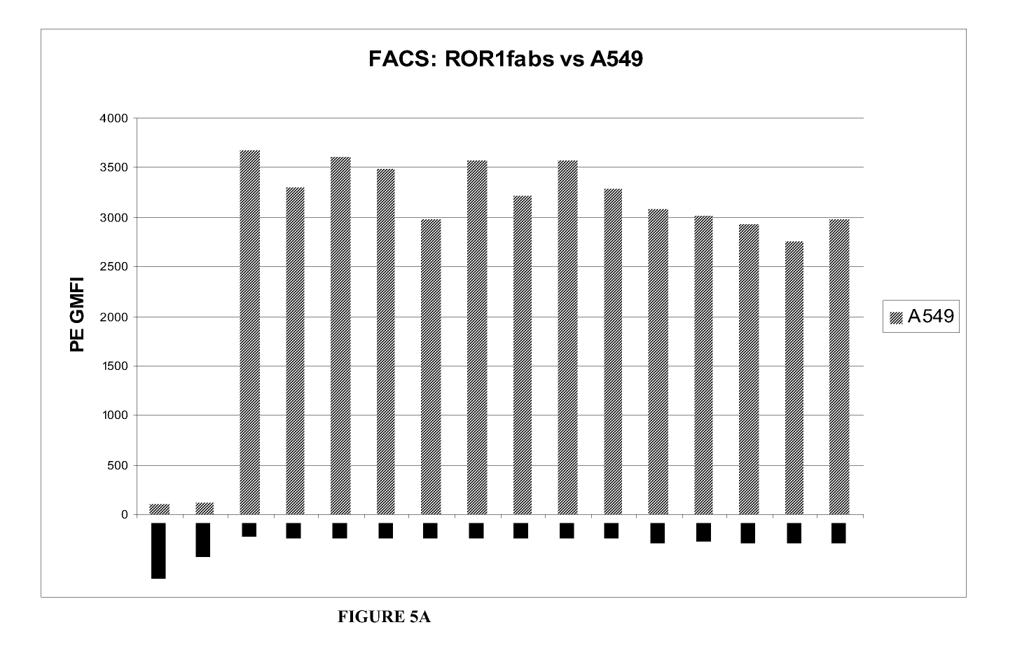

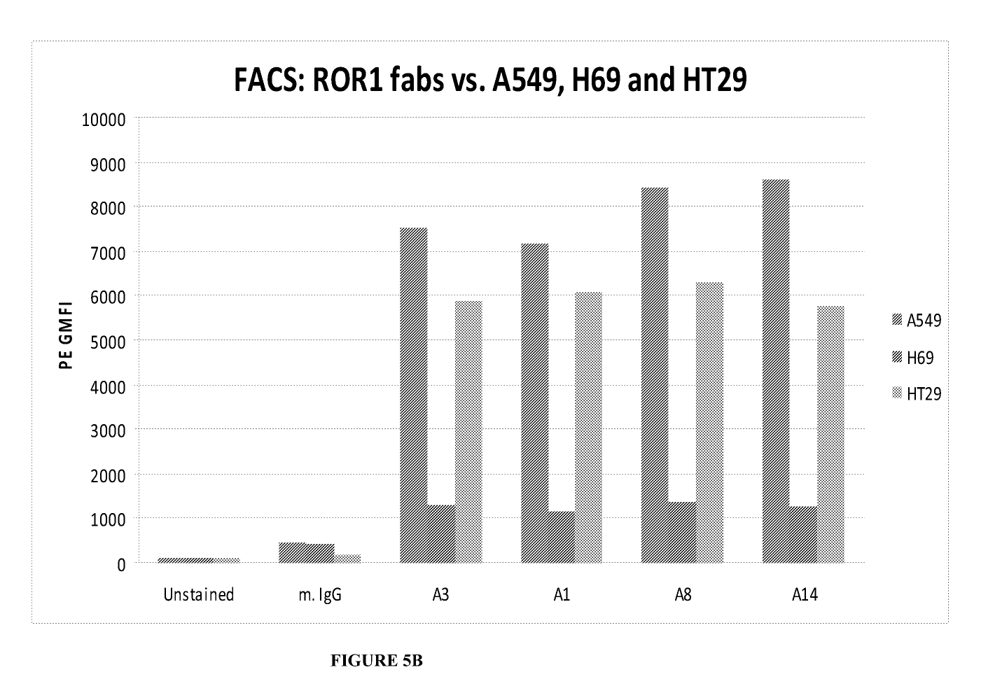

The results of the flow cytometry analysis demonstrated that 14 monoclonal antibodies designated ROR1_A1, ROR1_A2, ROR1_A3, ROR1_A4, ROR1_A5, ROR1_A6, ROR1_A7, ROR1_A8, ROR1 A9, ROR1_A10, ROR1_A11, ROR1_A12, ROR1_A13 and ROR1_A14 bound effectively to the cell-surface human ROR1 (FIG. 5a). FIG. 5b shows the binding specificities of ROR1_A3, ROR1_A1, ROR1 A8 and ROR1 A14 to ROR1 on A549, H69 and HT29 cell. The results indicate strong binding of those antibodies against ROR1 on A549 and HT29, but not on H69.

### Example 8

### Structural Characterization of Monoclonal Antibodies to Tyrosine-Protein Kinase Transmembrane Receptor ROR1

The cDNA sequences encoding the heavy and light chain variable regions of the ROR1_A1, ROR1_A2, ROR1_A3, ROR1_A4, ROR1_A5, ROR1_A6, ROR1_A7, ROR1_A8, ROR1_A9, ROR1_A10, ROR1_A11, ROR1_A12, ROR1_A13 and ROR1_A14 monoclonal antibodies were obtained using standard PCR techniques and were sequenced using standard DNA sequencing techniques.

The antibody sequences may be mutagenized to revert back to germline residues at one or more residues.

The nucleotide and amino acid sequences of the light chain variable region of ROR1_A1 are SEQ ID NO: 51 and 37, respectively.

The nucleotide and amino acid sequences of the heavy chain variable region of ROR1_A1 are SEQ ID NO: 23 and 9, respectively.

The nucleotide and amino acid sequences of the light chain variable region of ROR1_A2 are SEQ ID NO: 52 and 38, respectively.

The nucleotide and amino acid sequences of the heavy chain variable region of ROR1_A2 are SEQ ID NO: 24 and 10, respectively.

The nucleotide and amino acid sequences of the light chain variable region of ROR1_A3 are SEQ ID NO: 53 and 39, respectively.

The nucleotide and amino acid sequences of the heavy chain variable region of ROR1_A3 are SEQ ID NO: 25 and 11, respectively.

The nucleotide and amino acid sequences of the light chain variable region of ROR1_A4 are SEQ ID NO: 54 and 40, respectively.

The nucleotide and amino acid sequences of the heavy chain variable region of ROR1_A4 are SEQ ID NO: 26 and 12, respectively.

The nucleotide and amino acid sequences of the light chain variable region of ROR1_A5 are SEQ ID NO: 55 and 41, respectively.

The nucleotide and amino acid sequences of the heavy chain variable region of ROR1_A5 are SEQ ID NO: 27 and 13, respectively.

The nucleotide and amino acid sequences of the light chain variable region of ROR1_A6 are SEQ ID NO: 56 and 42, respectively.

The nucleotide and amino acid sequences of the heavy chain variable region of ROR1_A6 are SEQ ID NO: 28 and 14, respectively.

The nucleotide and amino acid sequences of the light chain variable region of ROR1_A7 are SEQ ID NO: 57 and 43, respectively.

The nucleotide and amino acid sequences of the heavy chain variable region of ROR1_A7 are SEQ ID NO: 29 and 15, respectively.

The nucleotide and amino acid sequences of the light chain variable region of ROR1_A8 are SEQ ID NO: 58 and 44, respectively.

The nucleotide and amino acid sequences of the heavy chain variable region of ROR1_A8 are SEQ ID NO: 30 and 16, respectively.

The nucleotide and amino acid sequences of the light chain variable region of ROR1_A9 are SEQ ID NO: 59 and 45, respectively.

The nucleotide and amino acid sequences of the heavy chain variable region of ROR1_A9 are SEQ ID NO: 31 and 17, respectively.

The nucleotide and amino acid sequences of the light chain variable region of ROR1_A10 are SEQ ID NO: 60 and 46, respectively.

The nucleotide and amino acid sequences of the heavy chain variable region of ROR1_A10 are SEQ ID NO: 32 and 18, respectively.

The nucleotide and amino acid sequences of the light chain variable region of ROR1_A11 are SEQ ID NO: 61 and 47, respectively.

The nucleotide and amino acid sequences of the heavy chain variable region of ROR1_A11 are SEQ ID NO: 33 and 19, respectively.

The nucleotide and amino acid sequences of the light chain variable region of ROR1_A12 are SEQ ID NO: 62 and 48, respectively.

The nucleotide and amino acid sequences of the heavy chain variable region of ROR1_A12 are SEQ ID NO: 34 and 20, respectively.

The nucleotide and amino acid sequences of the light chain variable region of ROR1_A13 are SEQ ID NO: 63 and 49, respectively.

The nucleotide and amino acid sequences of the heavy chain variable region of ROR1_A13 are SEQ ID NO: 35 and 21, respectively.

The nucleotide and amino acid sequences of the light chain variable region of ROR1_A14 are SEQ ID NO: 64 and 50, respectively.

The nucleotide and amino acid sequences of the heavy chain variable region of ROR1_A14 are SEQ ID NO: 36 and 22, respectively.

### Example 9

### Internalization of ROR1 all by HT29 Cells

ROR1_A11 was shown to be internalized by HT29 cells (human colon adenocarcinoma cell line) upon binding to the cells using an immunofluorescence microscopy assay. The immunofluorescence microscopy assay showed internalization of ROR1_A11 through binding of an anti-human IgG secondary antibody conjugated to fluorescein isothiocyanate (GamK-FITC).

The immunofluorescence microscopy assay was conducted as follows HT29 cells were incubated at 37° C. for 12 hr for cells to adhere to each other. ROR1 A11 and secondary antibody conjugated to fluorescein isothiocyanate were serially diluted, washed with FACS buffer (PBS, 2% FBS) and then added to the culture media. The media was then washed again with FACS buffer (PBS, 2% FBS) and incubated at 37° C., after which 200 μl 2% PFA was added. Coverslips were mounted using a 9 μl aqueous mounting media and the cells were then visualized at regular time intervals using Leica fluorescent microscope. Surface binding of ROR1_A11/secondary antibody FITC conjugate complex to HT29 cells was observed after 0 min, 15 min, 60 min and 120 min. The complete internalization of ROR1_A11 was observed after 120 min.

### Example 10

### Internalization and MabZAP of ROR1_A3, ROR1_A1, ROR1_A8 and ROR1_A14 in HEK293, H69 Ht-29 and A549 Cells

Internalization of ROR1_A3, ROR1_A1, ROR1_A8 and ROR1_A14 by HEK293 (human embryonic kidney), H69, HT29 and A549 were investigated using a MabZap assay. The MabZAP assay showed internalization of the anti-ROR1 monoclonal antibodies through binding of an anti-human IgG secondary antibody conjugated to the toxin saporin. (Advanced Targeting System, San Diego, Calif., IT-22-100). First, ROR1Fab was bound to the surface of the cells. Then, the MabZAP antibodies were bound to the primary antibodies. Next, the MabZAP complex was internalized by the cells. The entrance of Saporin into the cells resulted in protein synthesis inhibition and eventual cell death.

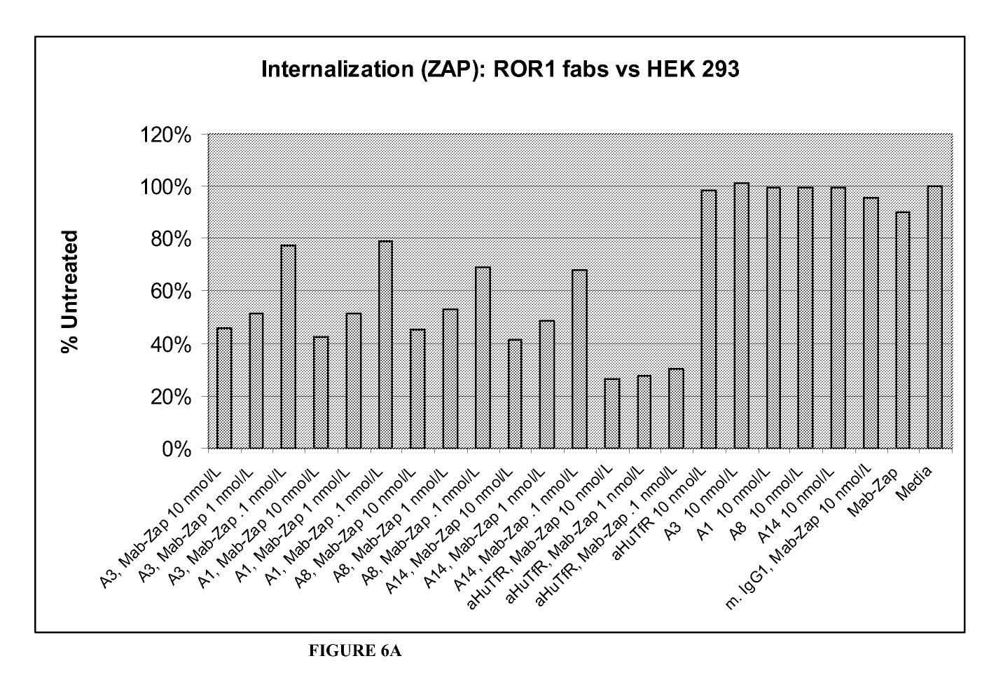

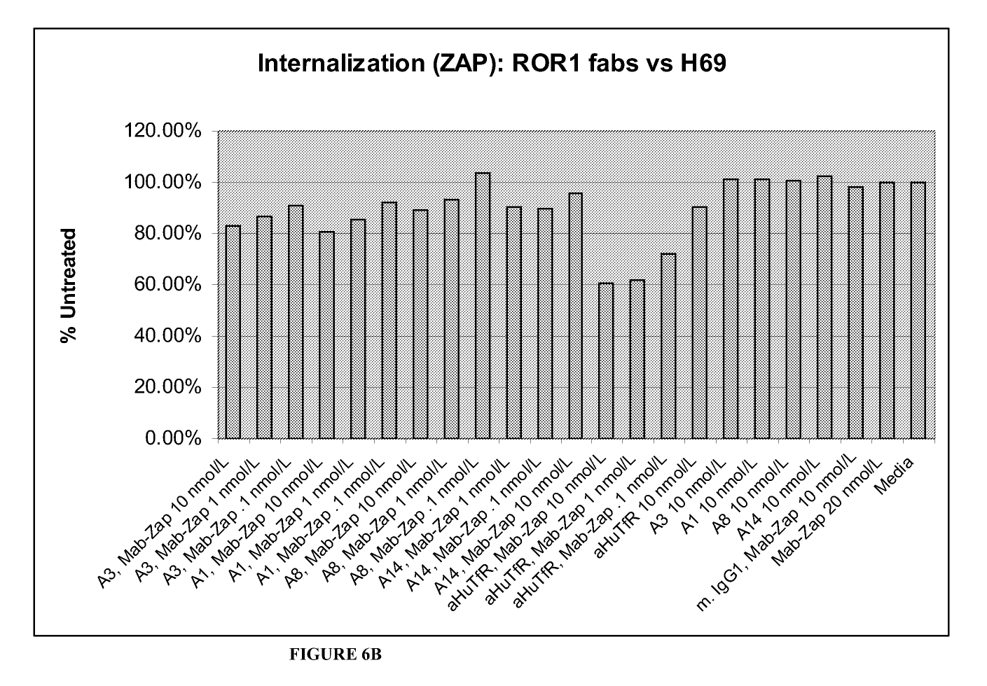

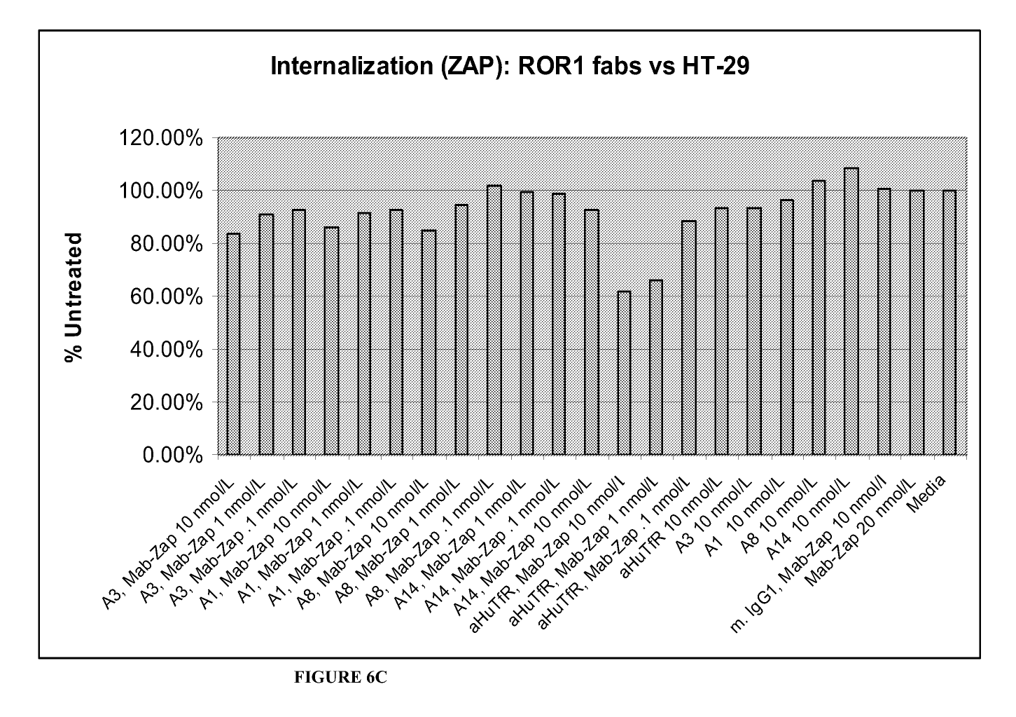

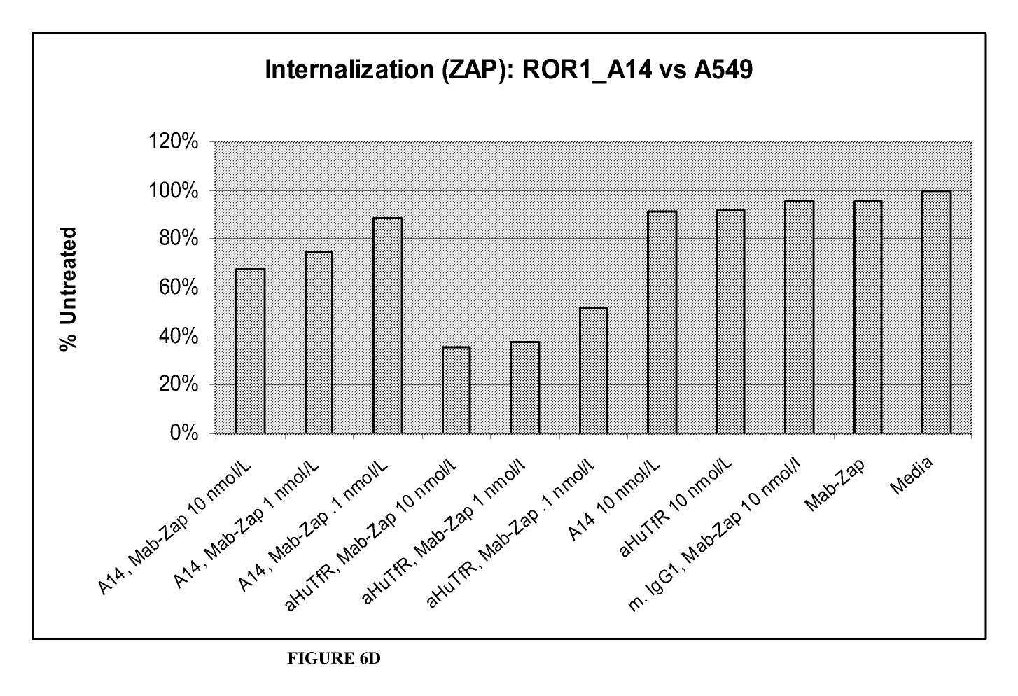

The MabZAP assay was conducted as follows. Each of the cells was seeded at a density of 5×10³ cells per well. The anti-ROR1 monoclonal antibody or an isotype control human IgG was serially diluted then added to the cells and incubated for 15 min at 25° C. The MabZAP was then added and incubated for 72 hr at 37° C. Cell viability in the plates was detected by CellTiter-Glo® Luminescent Cell Viability Assay kit (Promega, G7571) and the plates were read and analysed using Promega Glomax. Cell death was proportional to the concentration of anti-ROR1 monoclonal antibodies. FIGS. 6a to 6c show that the anti-ROR1 monoclonal antibodies, ROR1_A3, ROR1_A1, ROR1_A8 and ROR1_A14 were efficiently internalized by HEK293 cells and less efficiently by H69 and HT-29 cells, as compared to the anti-human IgG isotype control antibody. Internalization of ROR1_A14 was also observed by A549 cells (FIG. 6d).

### Discussion of the Examples

The proteomics data described in Examples 1 and 2 demonstrates that 3 tandem sequences and 5 different MALDI Peptides identifying ROR1 have been detected from the following malignant samples: pancreatic cancer, lung cancer, colon cancer, kidney cancer, breast cancer. Importantly these peptides are from the extra-cellular domain of ROR1. Furthermore no peptides from the ECD have been detected in a comprehensive survey of human normal tissues with the only detection of ROR1 being a peptide from the intracellular domain which was identified in a normal testis sample.

As shown by the proteomics studies, ROR1 can be detected in tumor samples in excess of normal tissues. Furthermore peptides discovered by these proteomics studies reside in the extracellular region of the ROR1 protein and are specific for cancer samples over normal tissue samples. Thus proteomics has defined a variant (splice or otherwise) of ROR1 that contains an extracellular domain specific to cancer samples. To further verify this discovery, RT-PCR analysis was performed on cancer tissues, cancer cell lines, and normal tissues using oligonucleotides that specifically amplify part of the proposed extra-cellular domain of ROR1. The results of these RT-PCR experiments clearly demonstrate increased presence of mRNA encoding the extracellular region of ROR1 in cancer samples over normal samples. High expression of this long variant of ROR1 which contains the extracellular domains is seen in the following clinical cancer samples: breast cancer, colon cancer, kidney cancer, liver cancer, lung cancer, stomach cancer, thyroid cancer, skin cancer (including melanoma). High expression is also seen in cell lines representing the following cancers: lung cancer—SCLC and NSCLC (A549, CALU1, CORL23, H226, H322, H358, H69), colon cancer (HCT116, HT29), and pancreatic cancer (PANC1).

As presented here, the extra-cellular domain of ROR1 represents a target of great potential for development of anti cancer therapeutics in multiple diseases. One preferred therapeutic is monoclonal antibodies which could elicit their anti cancer effects via different mechanisms of action including: ADCC, CDC, antibody-toxin-conjugates, functional modulation of the target, immune stimulation using e.g. bispecific antibodies co targeting CD3 and ROR1 or anti ROR1 antibodies fused to e.g. IL2. Combinations of these mechanisms may improve the anti cancer activity of anti ROR1 antibodies. Thus we sought to derive anti ROR1 antibodies specific to the ECD. Mice were immunized with eukaryotically expressed protein representing the ECD of ROR1. Phage libraries generated from these mice were bound in liquid phase and then plated to generate individual clones of ROR1-ECD reactive Antibodies. Following confirmation of binding to ROR1-ECD by ELISA, selected antibodies were tested for functionality as potential therapeutic candidates as below.

An important part of targeting cancer cells with antibodies is the ability of such antibodies to bind specifically to the cell surface of cancer cells. Antibodies screened for specificity to the extracellular region of ROR1 by ELISA were confirmed for their ability to bind to the cell surface of cancer cells by FACS. Different antibodies were shown to bind to the NSCLC cell line A549. Those showing the best (highest MFI signal) binding were subsequently shown to bind specifically and with significant intensity to the colon cancer cell line HT29 and the SCLC cell line NCI-H69 (FIG. 5b). These 3 cell lines are noted above as being positive for ROR1 extracellular domain by RT-PCR. Furthermore, these ROR1 antibodies were shown to specifically bind to CLL cells isolated from a patient demonstrating their potential utility in that disease setting.

For a target to be optimally targeted by cytolytic antibodies, the epitope on the target being bound by the antibodies should be available more so on cancer cells than on normal cells. As discussed elsewhere, there are long and short forms of ROR1. The antibodies described here are directed to the extracellular regions of ROR1 which is only present on the long form of the protein. The proteomics and RNA data described here support that the long form of ROR1 is not expressed significantly in normal tissues but is expressed in malignant tissues and cells. Thus, antibodies directed to the extracellular regions of ROR1 should stain cancer tissues more so than normal tissues which express the short form of ROR1 more than the long form of ROR1. Two different anti ROR1 antibodies show clear staining of cancer tissues and cells in two different IHC formats (frozen tissues and FFPE embedded tissues). For FFPE embedded sections specific staining of cancer cells was observed for breast cancer and NSCLC. No staining was observed in the same experiment for any normal tissues represented in a screen of the FDA recommended tissue set. Furthermore, within the cancer sections, NAT was not stained. Similar results were obtained using frozen sections where breast cancer and NSCLC stained positively and no staining was observed on the following normal tissues: heart, skin, colon, brain, kidney, liver, lung, lymph node, spleen, stomach. Thus the combination of the expression distribution of the different forms of ROR1 coupled with the specific design and screen for anti ROR1 antibodies provides methods for anti cancer antibody therapeutics.

A preferred mechanism for utilizing antibodies as cancer therapeutics where cancer specific binding can be achieved is as antibody-drug-conjugates (ADCs). Here it is preferable that the antibody/antigen complex internalizes into the cell from the plasma membrane after binding. Internalization of ROR1 and anti ROR1 antibodies is demonstrated by two different methods (FIG. 6). The data show that cancer cells can be killed by a MabZAP assay. Here the anti ROR1 antibody is mixed with viable cancer cells in growth media, along with a secondary anti mouse antibody which is conjugated to the toxin Saporin. The conjugated anti mouse antibody can join the complex with the anti ROR1 antibody/ROR1 protein, and if that complex is internalized into the cells will cause cell death which is measured by a viable cell assay. In this assay anti ROR1 antibodies specifically enable ZAP based cell kill on 4 different cell lines which express ROR1: HEK293 (embryonic kidney cells), HT29 (colon cancer cells), A549 (NSCLC cells), and NCI-H69 (SCLC cells). Thus the ROR1 antibodies are internalized. The kinetics of this internalization were explored on the HT29 (colon cancer) cells using immunofluorescence. Here, the anti ROR1 antibodies were allowed to bind to the cells at 0° C. The cells were then moved to 37° C. and the anti ROR1 antibodies tracked using a FITC labeled anti mouse antibody at different timepoints. Visualization using a fluorescent microscope showed distribution of the ROR1 protein/Ab complex as follows:

0 minutes, homogeneous cell surface, a ring is visible around the cell

15 minutes, the cell surface is still visible as a ring but the fluorescence is clustered in spots

60 minutes, a mixture of clustered fluorescence at the cell surface and puntate spots within the cell

120 minutes, no cell surface localization is visible, all fluorescence is puntate and intracellular

SEQUENCE LISTING

| Seq | | |
|---|---|---|
| ID | Description | Sequence |
| 1 | Tyrosine-protein | MHRPRRRGTRPPLLALLAALLLAARGAAAQETELSVSAELVPTSSWNISSELNKDSYLTLDEPMNNI |
| | kinase trans- | TTSLGQTAELHCKVSGNPPPTIRWFKNDAPVVQEPRRLSFRSTIYGSRLRIRNLDTTDTGYFQCVAT |
| | membrane receptor | NGKEVVSSTGVLFVKFGPPPTASPGYSDEYEEDGFCQPYRGIACARFIGNRTVYMESLHMQGEIEN |
| | ROR1 (Long | QITAAFTMIGTSSHLSDKCSQFAIPSLCHYAFPYCDETSSVPKPRDLCRDECEILENVLCQTEYIFARS |
| | Isoform) | NPMILMRLKLPNCEDLPQPESPEAANCIRIGIPMADPINKNHKCYNSTGVDYRGTVSVTKSGRQCQP |
| | | WNSQYPHTHTFTALRFPELNGGHSYCRNPGNQKEAPWCFTLDENFKSDLCDIPACDSKDSKEKNK |
| | | MEILYILVPSVAIPLAIALLFFFICVCRNNQKSSSAPVQRQPKHVRGQNVEMSMLNAYKPKSKAKELP |
| | | LSAVRFMEELGECAFGKIYKGHLYLPGMDHAQLVAIKTLKDYNNPQQWTEFQQEASLMAELHHPNI |
| | | VCLLGAVTQEQPVCMLFEYINQGDLHEFLIMRSPHSDVGCSSDEDGTVKSSLDHGDFLHIAIQIAAG |
| | | MEYLSSHFFVHKDLAARNILIGEQLHVKISDLGLSREIYSADYYRVQSKSLLPIRWMPPEAIMYGKFSS |
| | | DSDIWSFGVVLWEIFSFGLQPYYGFSNQEVIEMVRKRQLLPCSEDCPPRMYSLMTECWNEIPSRRP |
| | | RFKDIHVRLRSWEGLSSHTSSTTPSGGNATTQTTSLSASPVSNLSNPRYPNYMFPSQGITPQGQIA |
| | | GFIGPPIPQNQRFIPINGYPIPPGYAAFPAAHYQPTGPPRVIQHCPPPKSRSPSSASGSTSTGHVTSL |
| | | PSSGSNQEANIPLLPHMSIPNHPGGMGITVFGNKSQKPYKIDSKQASLLGDANIHGHTESMISAEL |
| 2 | ROR1 Peptide | EVVSSTGVLFVK |
| | ROR1 Peptide | GTRPPLLALLAALLLAAR |
| 4 | ROR1 Peptide | ELPLSAVR |
| 5 | ROR1 Peptide | SNPMILMR |
| 6 | ROR1 Peptide | SNPMILMRLK |
| 7 | ROR1 Peptide | GHLYLPGMDHAQLVAIK |
| 8 | Tyrosine-protein | QETELSVSAELVPTSSWNISSELNKDSYLTLDEPMNNITTSLGQTAELHCKVSGNPPPTIRWFKNDA |
| | kinase | PVVQEPRRLSFRSTIYGSRLRIRNLDTTDTGYFQCVATNGKEVVSSTGVLFVKFGPPPTASPGYSDE |
| | transmembrane | YEEDGFCQPYRGIACARFIGNRTVYMESLHMQGEIENQITAAFTMIGTSSHLSDKCSQFAIPSLCHYA |
| | receptor ROR1 | FPYCDETSSVPKPRDLCRDECEILENVLCQTEYIFARSNPMILMRLKLPNCEDLPQPESPEAANCIRI |
| | (ECD a.a 30-407 | GIPMADPINKNHKCYNSTGVDYRGTVSVTKSGRQCQPWNSQYPHTHTFTALRFPELNGGHSYCRN |
| | of long isoform) | PGNQKEAPWCFTLDENFKSDLCDIPACDSKDSKEKNKMEILYI |
| 9 | VH_amino acid A1 | MKQSTIALALLPLLFTPVAKAEVKLVESGGGLVRPGGSLKLSCAVSGFTFSSYAMSWVRQTPEKRLE |
| | | WVAAINFNRGTTYYSDTVKGRFTISRDNAKNTLYLQLSSLRSEDTAFYYCSRHRYSDYDYAMDYWG |
| | | QGTSVTVSSAKTTPPSVYPLAPGSAAQTNSMVTLGCLVKGYFPEPVTVTWNSGSLSSGVHTFPAVL |
| | | QSDLYTLSSSVTVPSSTWPSETVTCNVAHPASSTKVDKKIVPRDCHHHHHHH |
| 10 | VH_amino acid A2 | MKQSTIALALLPLLFTPVAKAEVQLLETGGGLVKPGGSLKLSCAASGFTFSTYAMSWVRQTPEKRLE |
| | | WVAGINSNRGTTYYPDTVKGRFTISRDNAKNTLSLQMTSLRSEDTALYYCVRHRYTNYDYAMDYW |
| | | GQGTSVTVSSAKTTPPSVYPLAPGSAAQTNSMVTLGCLVKGYFPEPVTVTWNSGSLSSGVHTFPA |
| | | VLQSDLYTLSSSVTVPSSTWPSETVTCNVAHPASSTKVDKKIVPRDCHHHHHHH |
| 11 | VH_amino acid A3 | MKQSTIALALLPLLFTPVAKADVMLVESGGGLVKPGGSLKLSCAASGFTFSSYAMSWVRQTPEKRL |
| | | EWVAAININRGTTYYSDTVKGRFTISRDNAKNTLYLQLSSLRSEDTALYYCSRHRYSDYDYAMDYW |
| | | GQGTSVTVSSAKTTPPSVYPLAPGSAAQTNSMVTLGCLVKGYFPEPVTVTWNSGSLSSGVHTFPA |
| | | VLQSDLYTLSSSVTVPSSTWPSETVTCNVAHPASSTKVDKKIVPRDCHHHHHHH |
| 12 | VH_amino acid A4 | MKQSTIALALLPLLFTPVAKAEVKLVESGGGLVKPGGSLKLSCAASGFTFSNYGMSWVRQTPERRL |
| | | EWVAAMNNNGASTYYPDTVKGRFTISRDNAKNTLYLQMSSLRSEDTALYFCVRHNNYVDYAMDYW |
| | | GQGTSVTVSSAKTTPPSVYPLAPGSAAQTNSMVTLGCLVKGYFPEPVTVTWNSGSLSSGVHTFPA |
| | | VLQSDLYTLSSSVTVPSSTWPSETVTCNVAHPASSTKVDKKIVPRDCHHHHHHH |
| 13 | VH_amino acid A5 | MKQSTIALALLPLLFTPVAKAEVKLVESGGGLVKPGGSLKLSGAASGFTFSNYDMSWVRQSPEKRL |
| | | EWVAAINRKGHSTYYPDTVQGRFTISRDNAKNTLYLQMSSLRSEDTALYYCVRLDDNYYFFDYWGQ |
| | | GTTLTVSSAKTTPPSVYPLAPGSAAQTNSMVTLGCLVKGYFPEPVTVTWNSGSLSSGVHTFPAVLQ |
| | | SDLYTLSSSVTVPSSTWPSETVTCNVAHPASSTKVDKKIVPRDCHHHHHHH |
| 14 | VH_amino acid A6 | MKQSTIALALLPLLFTPVAKAEVMLVESGGGLVKPGGSLKLSCAASGFTFSPYAMSWVRQTPEKRL |
| | | EWVAAINSNRGTTYYPDTVKGRFTISRDNAKNTLYLQMSSLRSEDTAFYYCVRHRYNNYDYAMDY |
| | | WGQGTSVTVSSAKTTPPSVYPLAPGSAAQTNSMVTLGCLVKGYFPEPVTVTWNSGSLSSGVHTFP |
| | | AVLQSDLYTLSSSVTVPSSTWPSETVTCNVAHPASSTKVDKKIVPRDCHHHHHHH |
| 15 | VH_amino acid A7 | MKQSTIALALLPLLFTPVAKAEVMLVESGGGLVKPGGSLKISCAASGFSFSSYAMSWVRQTPEKSLE |
| | | WVAAININRGTPYYPDTVKGRFTISRDNAKNTLYLQMSSLRSEDTALYYCVRHRNSNNDYAMDYWG |
| | | QGTSVTVSSAKTTPPSVYPLAPGSAAQTNSMVTLGCLVKGYFPEPVTVTWNSGSLSSGVHTFPAVL |
| | | QSDLYTLSSSVTVPSSTWPSETVTCNVAHPASSTKVDKKIVPRDCHHHHHHH |
| 16 | VH_amino acid A8 | MKQSTIALALLPLLFTPVAKADVQVVESGGGLVKPGGSLKLSGAASGFTFSSYAMSWVRQTPEKRL |
| | | EWVAAINPNGGSTYYPDTVKGRFTISRDNAKNTLYLQMSGLRSEDTALYYCARLPWSPYTLDYWG |
| | | QGTSVTVSSAKTTPPSVYPLAPGSAAQTNSMVTLGCLVKGYFPE |
| 17 | VH_amino acid A9 | MKQSTIALALLPLLFTPVAKAEVQLVETGGDLVKPGGSLKLSCVASGFTFSSNAMSWVRQTPEKRLE |
| | | WVAAINSKGGGTYYPDTVRGRFTISRDNAKNTLYLQVTSLRSEDTALYYCVSHGDNKYFYAMDYW |
| | | GQGTSVTVSSAKTTPPSVYPLAPGSAAQTNSMVTLGCLVKGYFPEPVTVTWNSGSLSSGVHTFPA |
| | | VLQSDLYTLSSSVTVPSSTWPSETVTCNVAHPASSTKVDKKIVPRDCHHHHHHH |
| 18 | VH_amino acid A10 | MKQSTIALALLPLLFTPVAKAEVQLVETGGGLVKPGGSLKLSCAASGFAFSSYAMSWVRQTPEKRLE |
| | | WVAAINNRGGGTYYPDTVRGRFTISRDNAKNTLYLQMSSLRSADTALYYCVRHDNLNYDYAMDSW |
| | | GQGTSVTVSSAKTTPPSVYPLAPGSAAQTNSMVTLGCLVKGYFPEPVTVTWNSGSLSSGVHTFPA |
| | | VLQSDLYTLSSSVTVPSSTWPSETVTCNVAHPASSTKVDKKIVPRDCHHHHHHH |
| 19 | VH_amino acid A11 | MKQSTIALALLPLLFTPVPKAEVQLVESGGDLVKPGGSLKLSCAASGFTFSRYGMSWVRQTPEKRL |
| | | EWVAAINPNGGTTYYPDTVKGRFTISRDNAKNTLFLQMTGLRSEDTALYYCARLPWSPYTLDYWGQ |
| | | GTSVIVSSAKTTPPSVYPLAPGSAAQTNSMVTLGCLVKGYFPEPVTVTWNSGSLSSGVHTFPAVLQ |
| | | SDLYTLSSSVTVPSSTWPSETVTCNVAHPASSTKVDKKIVPRDCHHHHHHH |
| 20 | VH_amino acid A12 | MKQSTIALALLPLLFTPVAKAEVQLVESGGGLVKPGGSLKLSCAASGFTFSSYAMSWVRQTPEKRLE |
| | | WVAAINSNRGTTYYSDTVKGRFTISRDNAKNTLYLQMSSLRSEDTAFYYCTRHRYSDYDYAMDYW |
| | | GQGTSVTVSSAKTTPPSVYPLAPGSAAQTNSMVTLGCLVKGYFPEPVTVTWNSGSLSSGVHTFPA |
| | | VLQSDLYTLSSSVTVPSSTWPSETVTCNVAHPASSTKVDKKIVPRDCHHHHHHH |
| 21 | VH_amino acid A13 | MKQSTIALALLPLLFTPVAKAEVQLVETGGGLVKPGGSLKLSCAASGFTFSSYAMSWIRQTPEKRLE |
| | | WVAGINSNRGTTYYPDTVKGRFTISRDNAKNTLYLQMNSLRSEDSALYYCVRHRYIDYDYAMDYWG |
| | | QGTSVTVSSAKTTPPSVYPLAPGSAAQTNSMVTLGCLVKGYFPEPVTVTWNSGSLSSGVHTFPAVL |
| | | QSDLYTLSSSVTVPSSTWPSETVTCNVAHPASSTKVDKKIVPRDCHHHHHHH |
| 22 | VH_amino acid A14 | MKQSTIALALLPLLFTPVAKAQVQLKQSGAELVKPGASVKISCKATGYTFSSYWIEWVKERPGHGLE |
| | | WIGEILPGIGNTNYNEKFKGKATFTADLSSKTAYMQLSSLTSEDSAVYYCASGGYSTVYWYFDVWG |
| | | AGTTVTVSSAKTTPPSVYPLAPGSAAQTNSMVTLGCLVKGYFPEPVTVTWNSGSLSSGVHTFPAVL |
| | | QSDLYTLSSSVTVPSSTWPSETVTCNVAHPASSTKVDKKIVPRDCHHHHHHH |
| 23 | VH_nt A1 | TTACCCACGCTTTGTACATGGAGAAAATAAAGTGAAACAAAGCACTATTGCACTGGCACTCTTAC |
| | | CGCTCTTATTTACCCCTGTGGCAAAAGCCGAGGTGAAGCTGGTGGAATCTGGGGGAGGCTTAG |
| | | TGAGGCCTGGAGGGTCCCTGAAACTCTCCTGTGCAGTCTCTGGATTCACTTTCAGTAGCTATGC |
| | | CATGTCTTGGGTTCGCCAGACTCCGGAGAAGAGGCTGGAATGGGTCGCAGCCATTAATTTTAAT |
| | | CGTGGTACCACCTACTATTCAGACACTGTGAAGGGCCGATTCACCATCTCCAGAGACAATGCCA |
| | | AGAATACCCTGTACCTGCAACTGAGCAGTCTGAGGTCTGAGGACACAGCCTTTTATTACTGTTC |
| | | AAGACACCGCTATAGTGACTACGACTATGCTATGGACTACTGGGGTCAAGGAACCTCAGTCACC |
| | | GTCTCCTCAGCCAAAACGACACCCCCATCTGTCTATCCACTGGCCCCTGGATCTGCTGCCCAAA |
| | | CTAACTCCATGGTGACCCTGGGATGCCTGGTCAAGGGCTATTTCCCTGAGCCAGTGACAGTGA |
| | | CCTGGAACTCTGGATCCCTGTCCAGCGGTGTGCACACCTTCCCAGCTGTCCTGCAGTCTGACC |
| | | TCTACACTCTGAGCAGCTCAGTGACTGTCCCCTCCAGCACCTGGCCCAGCGAGACCGTCACCT |
| | | GCAACGTTGCCCACCCGGCCAGCAGCACCAAGGTGGACAAGAAAATTGTGCCCAGGGATTGTC |
| | | ATCATCACCATCACCATCACTAAATGGACAGCTTAATCATTTATAAAGCT |
| 24 | VH_nt A2 | AACCCTGGCGTTACCCACGCTTTGTACATGGAGAAAATAAAGTGAAACAAAGCACTATTGCACT |
| | | GGCACTCTTACCGCTCTTATTTACCCCTGTGGCAAAAGCCGAAGTGCAGCTGTTGGAGACTGG |
| | | GGGAGGCTTAGTGAAGCCTGGAGGGTCCCTGAAACTCTCCTGTGCAGCCTCTGGATTCACTTT |
| | | CAGTACCTATGCCATGTCTTGGGTTCGCCAGACTCCGGAGAAGAGGCTGGAGTGGGTCGCAG |
| | | GCATTAATAGTAATCGTGGTACCACCTACTATCCAGACACTGTGAAGGGCCGCTTCACCATCTC |
| | | CAGAGACAATGCCAAGAACACCCTGTCCCTGCAAATGACCAGTCTGAGGTCTGAGGACACAGC |
| | | CTTGTATTATTGTGTAAGACACCGCTATACTAACTACGACTATGCTATGGACTACTGGGGTCAAG |
| | | GAACCTCAGTCACCGTCTCCTCAGCCAAAACGACACCCCCATCTGTCTATCCACTGGCCCCTG |
| | | GATCTGCTGCCCAAACTAACTCCATGGTGACCCTGGGATGCCTGGTCAAGGGCTATTTCCCTG |
| | | AGCCAGTGACAGTGACCTGGAACTCTGGATCCCTGTCCAGCGGTGTGCACACCTTCCCAGCTG |
| | | TCCTGCAGTCTGACCTCTACACTCTGAGCAGCTCAGTGACTGTCCCCTCCAGCACCTGGCCCA |
| | | GCGAGACCGTCACCTGCAACGTTGCCCACCCGGCCAGCAGCACCAAGGTGGACAAGAAAATT |
| | | GTGCCCAGGGATTGTCATCATCACCATCACCATCACTAATTGACAGCTTATCATCGATAAGCTTT |
| | | AATGCGGTAGTTTAT |
| 25 | VH_nt A3 | CGTCGTTTTACAACGTCGTGACTGGGAAAACCCTGGCGTTACCCACGCTTTGTACATGGAGAAA |
| | | ATAAAGTGAAACAAAGCACTATTGCACTGGCACTCTTACCGCTCTTATTTACCCCTGTGGCAAAA |
| | | GCCGATGTGATGCTGGTGGAGTCTGGGGGAGGCTTAGTGAAGCCTGGAGGGTCCCTGAAACT |
| | | CTCCTGTGCAGCCTCTGGATTCACTTTCAGTAGCTATGCCATGTCTTGGGTTCGCCAGACTCCG |
| | | GAGAAGAGGCTGGAATGGGTCGCAGCCATTAATATTAATCGTGGTACCACCTACTATTCAGACA |
| | | CTGTGAAGGGCCGATTCACCATCTCCAGAGACAATGCCAAGAATACCCTGTACCTGCAACTGA |
| | | GCAGTCTGAGGTCTGAGGACACAGCCTTGTATTACTGTTCAAGACACCGCTATAGTGACTACGA |
| | | CTATGCTATGGACTACTGGGGTCAAGGAACCTCAGTCACCGTCTCCTCAGCCAAAACGACACC |
| | | CCCATCTGTCTATCCACTGGCCCCTGGATCTGCTGCCCAAACTAACTCCATGGTGACCCTGGG |
| | | ATGCCTGGTCAAGGGCTATTTCCCTGAGCCAGTGACAGTGACCTGGAACTCTGGATCCCTGTC |
| | | CAGCGGTGTGCACACCTTCCCAGCTGTCCTGCAGTCTGACCTCTACACTCTGAGCAGCTCAGT |
| | | GACTGTCCCCTCCAGCACCTGGCCCAGCGAGACCGTCACCTGCAACGTTGCCCACCCGGCCA |
| | | GCAGCACCAAGGTGGACAAGAAAATTGTGCCCAGGGATTGTCATCATCACCATCACCATCACTA |
| | | ATTGACAGCTTATCATCGATAAGCTTTAATGCGGTAGTTT |
| 26 | VH_nt A4 | TTACCCACGCTTTGTACATGGAGAAAATAAAGTGAAACAAAGCACTATTGCACTGGCACTCTTAC |
| | | CGCTCTTATTTACCCCTGTGGCAAAAGCCGAGGTGAAGCTGGTGGAATCTGGGGGAGGCTTAG |
| | | TGAAGCCTGGAGGGTCCCTGAAACTCTCCTGTGCAGCCTCTGGATTCACTTTCAGTAACTATGG |
| | | CATGTCTTGGGTTCGCCAGACTCCGGAGAGGAGGCTGGAGTGGGTCGCAGCCATGAATAATAA |
| | | TGGTGCTAGCACCTACTATCCAGACACTGTGAAGGGCCGATTCACCATCTCCAGAGACAATGC |
| | | CAAGAACACCCTGTACCTGCAAATGAGCAGTCTGAGGTCTGAGGACACAGCCTTGTATTTCTGT |
| | | GTAAGACATAATAACTACGTTGACTATGCTATGGACTATTGGGGTCAAGGAACCTCAGTCACCG |
| | | TCTCCTCAGCCAAAACGACACCCCCATCTGTCTATCCACTGGCCCCTGGATCTGCTGCCCAAAC |
| | | TAACTCCATGGTGACCCTGGGATGCCTGGTCAAGGGCTATTTCCCTGAGCCAGTGACAGTGAC |
| | | CTGGAACTCTGGATCCCTGTCCAGCGGTGTGCACACCTTCCCAGCTGTCCTGCAGTCTGACCT |
| | | CTACACTCTGAGCAGCTCAGTGACTGTCCCCTCCAGCACCTGGCCCAGCGAGACCGTCACCTG |
| | | CAACGTTGCCCACCCGGCCAGCAGCACCAAGGTGGACAAGAAAATTGTGCCCAGGGATTGTCA |
| | | TCATCACCATCACCATCACTAATTGACAGCTTATCATCGATAAGCTTTAATGCGGTAGTTTATCA |
| | | CAGTTAAATT |
| 27 | VH_nt A5 | ACCCTGGCGTTACCCACGCTTTGTACATGGAGAAAATAAAGTGAAACAAAGCACTATTGCACTG |
| | | GCACTCTTACCGCTCTTATTTACCCCTGTGGCAAAAGCCGAGGTGAAGCTGGTGGAATCTGGG |
| | | GGAGGCTTAGTGAAGCCTGGAGGGTCCCTGAAACTCTCCTGTGCAGCCTCTGGATTCACTTTC |
| | | AGTAACTATGACATGTCTTGGGTTCGCCAGAGTCCGGAGAAGAGGCTGGAGTGGGTCGCAGCC |
| | | ATTAATCGTAAAGGTCATAGTACCTACTATCCAGACACTGTGCAGGGCCGATTCACCATCTCCA |
| | | GAGACAATGCCAAGAACACCCTGTACCTGCAAATGAGCAGTCTGAGGTCTGAGGACACAGCCT |
| | | TGTATTACTGTGTAAGACTTGACGATAACTACTACTTCTTTGACTACTGGGGCCAAGGCACCACT |
| | | CTCACAGTCTCCTCAGCCAAAACGACACCCCCATCTGTCTATCCACTGGCCCCTGGATCTGCTG |
| | | CCCAAACTAACTCCATGGTGACCCTGGGATGCCTGGTCAAGGGCTATTTCCCTGAGCCAGTGA |
| | | CAGTGACCTGGAACTCTGGATCCCTGTCCAGCGGTGTGCACACCTTCCCAGCTGTCCTGCAGT |
| | | CTGACCTCTACACTCTGAGCAGCTCAGTGACTGTCCCCTCCAGCACCTGGCCCAGCGAGACCG |
| | | TCACCTGCAACGTTGCCCACCCGGCCAGCAGCACCAAGGTGGACAAGAAAATTGTGCCCAGG |
| | | GATTGTCATCATCACCATCACCATCACTAATTGACAGCTTATCATCGATAAGCTTTAATGCGGTA |
| | | GTTTAT |
| 28 | VH_nt A6 | GGAAAACCCTGGCGTTACCCACGCTTTGTACATGGAGAAAATAAAGTGAAACAAAGCACTATTG |
| | | CACTGGCACTCTTACCGCTCTTATTTACCCCTGTGGCAAAAGCCGAAGTGATGCTGGTGGAGTC |
| | | TGGGGGAGGCTTAGTGAAGCCTGGAGGGTCCCTGAAACTCTCCTGCGCAGCCTCTGGATTCAC |
| | | TTTCAGTCCCTATGCCATGTCTTGGGTTCGCCAGACTCCGGAGAAGAGGCTGGAGTGGGTCGC |
| | | AGCCATTAATAGTAATCGTGGTACCACCTACTATCCAGACACTGTGAAGGGCCGATTCACCATC |
| | | TCCAGAGACAATGCCAAGAACACCCTGTACCTGCAAATGAGCAGTCTGAGGTCTGAGGACACA |
| | | GCCTTTTATTACTGTGTAAGACACCGCTATAATAACTACGACTATGCTATGGACTACTGGGGTCA |
| | | AGGAACCTCAGTCACCGTCTCCTCAGCCAAAACGACACCCCCATCTGTCTATCCACTGGCCCCT |
| | | GGATCTGCTGCCCAAACTAACTCCATGGTGACCCTGGGATGCCTGGTCAAGGGCTATTTCCCT |
| | | GAGCCAGTGACAGTGACCTGGAACTCTGGATCCCTGTCCAGCGGTGTGCACACCTTCCCAGCT |
| | | GTCCTGCAGTCTGACCTCTACACTCTGAGCAGCTCAGTGACTGTCCCCTCCAGCACCTGGCCC |
| | | AGCGAGACCGTCACCTGCAACGTTGCCCACCCGGCCAGCAGCACCAAGGTGGACAAGAAAATT |
| | | GTGCCCAGGGATTGTCATCATCACCATCACCATCACTAATTGACAGCTTATCATCGATAAGCTTT |
| | | AATGCGGTAGTTTATCACAGT |
| 29 | VH_nt A7 | CGTTTTACAACGTCGTGACTGGGAAAACCCTGGCGTTACCCACGCTTTGTACATGGAGAAAATA |
| | | AAGTGAAACAAAGCACTATTGCACTGGCACTCTTACCGCTCTTATTTACCCCTGTGGCAAAAGC |
| | | CGAAGTGATGCTGGTGGAGTCTGGGGGAGGCTTAGTGAAGCCTGGAGGGTCCCTGAAAATCT |
| | | CCTGTGCAGCCTCTGGATTCTCTTTCAGTAGCTATGCCATGTCTTGGGTTCGCCAGACTCCGGA |
| | | GAAGAGCCTGGAATGGGTCGCAGCCATTAATATTAATCGTGGTACCCCCTATTATCCAGACACT |
| | | GTGAAGGGCCGATTCACCATCTCCAGAGACAATGCCAAGAACACCCTGTACCTGCAAATGAGT |
| | | AGTCTGAGGTCTGAGGACACAGCCTTGTATTACTGTGTAAGACACCGCAATAGTAACAACGACT |
| | | ATGCTATGGACTACTGGGGTCAAGGAACCTCAGTCACCGTCTCCTCAGCCAAAACGACACCCC |
| | | CATCTGTCTATCCACTGGCCCCTGGATCTGCTGCCCAAACTAACTCCATGGTGACCCTGGGATG |
| | | CCTGGTCAAGGGCTATTTCCCTGAGCCAGTGACAGTGACCTGGAACTCTGGATCCCTGTCCAG |
| | | CGGTGTGCACACCTTCCCAGCTGTCCTGCAGTCTGACCTCTACACTCTGAGCAGCTCAGTGAC |
| | | TGTCCCCTCCAGCACCTGGCCCAGCGAGACCGTCACCTGCAACGTTGCCCACCCGGCCAGCA |
| | | GCACCAAGGTGGACAAGAAAATTGTGCCCAGGGATTGTCATCATCACCATCACCATCACTAATT |
| | | GACAGCTTATCATCGATAAGCTTTAAT |
| 30 | VH_nt A8 | TACAACGTCGTGACTGGGAAAACCCTGGCGTTACCCACGCTTTGTACATGGAGAAAATAAAGTG |
| | | AAACAAAGCACTATTGCACTGGCACTCTTACCGCTCTTATTTACCCCTGTGGCAAAAGCCGACG |
| | | TGCAGGTGGTGGAGTCTGGGGGAGGCTTAGTGAAGCCTGGAGGGTCCCTGAAACTCTCCTGT |
| | | GCAGCCTCTGGATTCACTTTCAGTAGCTATGCCATGTCTTGGGTTCGCCAGACTCCGGAGAAGA |
| | | GGCTGGAGTGGGTCGCAGCCATTAATCCTAATGGTGGTAGTACCTACTATCCAGACACTGTGAA |
| | | GGGCCGATTCACCATCTCCAGAGACAATGCCAAGAACACCCTATACCTGCAAATGAGCGGTCT |
| | | GAGGTCTGAGGACACAGCCTTGTATTACTGTGCAAGACTCCCATGGTCCCCCTATACTTTGGAC |
| | | TACTGGGGTCAAGGAACCTCAGTCACCGTCTCCTCAGCCAAAACGACACCCCCATCTGTCTATC |
| | | CACTGGCCCCTGGATCTGCTGCCCAAACTAACTCCATGGTGACCCTGGGATGCCTGGTCAAGG |
| | | GCTATTTCCCTGAGC |
| 31 | VH_nt A9 | TTACCCACGCTTTGTACATGGAGAAAATAAAGTGAAACAAAGCACTATTGCACTGGCACTCTTAC |
| | | CGCTCTTATTTACCCCTGTGGCAAAAGCCGAAGTGCAGCTTGTGGAGACTGGGGGAGACTTAG |
| | | TGAAGCCTGGAGGGTCCCTGAAACTCTCCTGTGTAGCCTCTGGATTCACTTTCAGTAGCAATGC |
| | | CATGTCCTGGGTTCGCCAGACTCCGGAGAAGAGGCTGGAGTGGGTCGCAGCCATTAATAGTAA |
| | | AGGTGGTGGCACCTACTATCCAGACACTGTGAGGGGCCGATTCACCATCTCCAGAGACAATGC |
| | | CAAGAACACCCTGTACCTGCAAGTGACCAGTCTGAGGTCTGAGGACACAGCCTTGTATTACTGT |
| | | GTAAGCCATGGGGATAATAAGTACTTTTATGCTATGGACTACTGGGGTCAAGGAACCTCAGTCA |
| | | CCGTCTCCTCAGCCAAAACGACACCCCCATCTGTCTATCCACTGGCCCCTGGATCTGCTGCCC |
| | | AAACTAACTCCATGGTGACCCTGGGATGCCTGGTCAAGGGCTATTTCCCTGAGCCAGTGACAG |
| | | TGACCTGGAACTCTGGATCCCTGTCCAGCGGTGTGCACACCTTCCCAGCTGTCCTGCAGTCTG |
| | | ACCTCTACACTCTGAGCAGCTCAGTGACTGTCCCCTCCAGCACCTGGCCCAGCGAGACCGTCA |
| | | CCTGCAACGTTGCCCACCCGGCCAGCAGCACCAAGGTGGACAAGAAAATTGTGCCCAGGGATT |
| | | GTCATCATCACCATCACCATCACTAATTGACAGCTTATC |
| 32 | VH_nt A10 | TTACCCACGCTTTGTACATGGAGAAAATAAAGTGAAACAAAGCACTATTGCACTGGCACTCTTAC |
| | | CGCTCTTATTTACCCCTGTGGCAAAAGCCGAAGTGCAGCTTGTGGAGACTGGGGGAGGCTTAG |
| | | TGAAGCCTGGAGGGTCCCTGAAACTCTCCTGTGCAGCCTCTGGATTCGCTTTCAGTAGCTATGC |
| | | CATGTCTTGGGTTCGCCAAACTCCGGAGAAGAGGCTGGAGTGGGTCGCAGCCATTAATAATAG |
| | | AGGTGGTGGCACCTACTATCCAGACACTGTGAGGGGCCGATTCACCATCTCCAGAGACAATGC |
| | | CAAGAACACCCTGTACCTGCAAATGAGCAGCCTGAGGTCTGCGGACACAGCCTTGTATTACTGT |
| | | GTGAGACATGACAATCTTAACTATGACTATGCTATGGACTCCTGGGGTCAAGGAACCTCAGTCA |
| | | CCGTCTCCTCAGCCAAAACGACACCCCCATCTGTCTATCCACTGGCCCCTGGATCTGCTGCCC |
| | | AAACTAACTCCATGGTGACCCTGGGATGCCTGGTCAAGGGCTATTTCCCTGAGCCAGTGACAG |
| | | TGACCTGGAACTCTGGATCCCTGTCCAGCGGTGTGCACACCTTCCCAGCTGTCCTGCAGTCTG |
| | | ACCTCTACACTCTGAGCAGCTCAGTGACTGTCCCCTCCAGCACCTGGCCCAGCGAGACCGTCA |
| | | CCTGCAACGTTGCCCACCCGGCCAGCAGCACCAAGGTGGACAAGAAAATTGTGCCCAGGGATT |
| | | GTCATCATCACCATCACCATCACTAATTGACAGCTTATCATCGATAAGCTTTAA |
| 33 | VH_nt A11 | CGTCGTGACTGGGAAAACCCTGGCGTTACCCACGCTTTGTACATGGAGAAAATAAAGTGAAACA |
| | | AAGCACTATTGCACTGGCACTCTTACCGCTCTTATTTACCCCTGTGCCAAAAGCCGAAGTGCAG |
| | | CTGGTGGAGTCTGGGGGAGACTTAGTGAAGCCTGGAGGGTCCCTGAAACTCTCCTGTGCAGC |
| | | CTCTGGATTCACTTTCAGTAGATATGGCATGTCTTGGGTTCGCCAGACTCCGGAGAAGAGGCTG |
| | | GAGTGGGTCGCAGCCATTAATCCTAATGGTGGTACTACCTACTATCCAGACACTGTGAAGGGC |
| | | CGATTCACCATCTCCCGAGACAATGCCAAGAACACCCTGTTCCTGCAAATGACCGGTCTGAGGT |
| | | CTGAGGACACAGCCTTATACTACTGTGCAAGACTCCCATGGTCCCCCTATACTTTGGACTACTG |
| | | GGGTCAAGGAACCTCAGTCATCGTCTCCTCAGCCAAAACGACACCCCCATCTGTCTATCCACTG |
| | | GCCCCTGGATCTGCTGCCCAAACTAACTCCATGGTGACCCTGGGATGCCTGGTCAAGGGCTAT |
| | | TTCCCTGAGCCAGTGACAGTGACCTGGAACTCTGGATCCCTGTCCAGCGGTGTGCACACCTTC |
| | | CCAGCTGTCCTGCAGTCTGACCTCTACACTCTGAGCAGCTCAGTGACTGTCCCCTCCAGCACC |
| | | TGGCCCAGCGAGACCGTCACCTGCAACGTTGCCCACCCGGCCAGCAGCACCAAGGTGGACAA |
| | | GAAAATTGTGCCCAGGGATTGTCATCATCACCATCACCATCACTAATTGACAGCTTATCATCGAT |
| | | AAGCTTTAATGCGGTAGTTTATCACAGTTAAAT |
| 34 | VH_nt A12 | GCGTTACCCACGCTTTGTACATGGAGAAAATAAAGTGAAACAAAGCACTATTGCACTGGCACTC |
| | | TTACCGCTCTTATTTACCCCTGTGGCAAAAGCCGAAGTGCAGCTGGTGGAGTCTGGGGGAGGC |
| | | TTAGTGAAGCCTGGAGGGTCCCTGAAACTCTCCTGCGCAGCCTCTGGATTCACTTTCAGTAGCT |
| | | ATGCCATGTCTTGGGTTCGCCAGACTCCGGAGAAGAGGCTAGAGTGGGTCGCAGCCATTAATA |
| | | GTAATCGTGGTACCACCTACTATTCAGACACTGTGAAGGGCCGATTCACCATCTCCAGAGACAA |
| | | TGCCAAGAACACCCTGTACCTGCAAATGAGCAGTCTGAGGTCTGAGGACACAGCCTTCTATTAC |
| | | TGTACAAGACACCGCTATAGTGACTACGACTATGCTATGGACTACTGGGGTCAAGGAACCTCAG |
| | | TCACCGTCTCCTCAGCCAAAACGACACCCCCATCTGTCTATCCACTGGCCCCTGGATCTGCTGC |
| | | CCAAACTAACTCCATGGTGACCCTGGGATGCCTGGTCAAGGGCTATTTCCCTGAGCCAGTGAC |
| | | AGTGACCTGGAACTCTGGATCCCTGTCCAGCGGTGTGCACACCTTCCCAGCTGTCCTGCAGTC |
| | | TGACCTCTACACTCTGAGCAGCTCAGTGACTGTCCCCTCCAGCACCTGGCCCAGCGAGACCGT |
| | | CACCTGCAACGTTGCCCACCCGGCCAGCAGCACCAAGGTGGACAAGAAAATTGTGCCCAGGG |
| | | ATTGTCATCATCACCATCACCATCACTAATTGACAGCTTATCATCGATAAGCTTTAATGCGGTAG |
| | | TTTATCACAGTTAAATTGCTACG |
| 35 | VH_nt A13 | GGCGTTACCCACGCTTTGTACATGGAGAAAATAAAGTGAAACAAAGCACTATTGCACTGGCACT |
| | | CTTACCGCTCTTATTTACCCCTGTGGCAAAAGCCGAAGTGCAGCTTGTGGAGACTGGGGGAGG |
| | | CTTAGTGAAGCCTGGAGGGTCCCTGAAACTCTCCTGTGCAGCCTCTGGATTCACTTTCAGTAGC |
| | | TATGCCATGTCTTGGATTCGCCAGACTCCGGAGAAGAGGCTGGAGTGGGTCGCAGGCATTAAT |
| | | AGTAATCGTGGTACCACCTACTATCCAGACACTGTGAAGGGCCGATTCACCATCTCCAGAGACA |
| | | ATGCCAAGAACACCCTGTACCTGCAAATGAACAGTCTGAGGTCTGAGGACTCAGCCTTGTATTA |
| | | CTGTGTAAGACACCGCTATATTGACTACGACTATGCTATGGACTACTGGGGTCAAGGAACCTCA |
| | | GTCACCGTCTCCTCAGCCAAAACGACACCCCCATCTGTCTATCCACTGGCCCCTGGATCTGCT |
| | | GCCCAAACTAACTCCATGGTGACCCTGGGATGCCTGGTCAAGGGCTATTTCCCTGAGCCAGTG |
| | | ACAGTGACCTGGAACTCTGGATCCCTGTCCAGCGGTGTGCACACCTTCCCAGCTGTCCTGCAG |
| | | TCTGACCTCTACACTCTGAGCAGCTCAGTGACTGTCCCCTCCAGCACCTGGCCCAGCGAGACC |
| | | GTCACCTGCAACGTTGCCCACCCGGCCAGCAGCACCAAGGTGGACAAGAAAATTGTGCCCAG |
| | | AGTT |
| 36 | VH_nt A14 | GTCGTGACTGGGAAAACCCTGGCGTTACCCACGCTTTGTACATGGAGAAAATAAAGTGAAACAA |
| | | AGCACTATTGCACTGGCACTCTTACCGCTCTTATTTACCCCTGTGGCAAAAGCCCAGGTGCAGC |
| | | TTAAGCAGTCTGGGGCTGAGCTGGTGAAGCCTGGGGCCTCAGTGAAGATATCCTGCAAGGCTA |
| | | CTGGCTACACATTCAGTAGTTACTGGATAGAGTGGGTAAAGGAGAGGCCTGGACATGGCCTTG |
| | | AGTGGATTGGAGAGATTTTACCTGGAATTGGTAATACTAACTACAATGAGAAATTCAAGGGCAA |
| | | GGCCACATTCACTGCTGATCTATCCTCCAAGACAGCCTACATGCAACTCAGCAGCCTGACATCT |
| | | GAGGACTCTGCCGTCTATTACTGTGCAAGTGGGGGGTATAGTACCGTCTATTGGTATTTTGATG |
| | | TCTGGGGCGCAGGGACCACGGTCACCGTCTCCTCAGCCAAAACGACACCCCCATCTGTCTATC |
| | | CACTGGCCCCTGGATCTGCTGCCCAAACTAACTCCATGGTGACCCTGGGATGCCTGGTCAAGG |
| | | GCTATTTCCCTGAGCCAGTGACAGTGACCTGGAACTCTGGATCCCTGTCCAGCGGTGTGCACA |
| | | CCTTCCCAGCTGTCCTGCAGTCTGACCTCTACACTCTGAGCAGCTCAGTGACTGTCCCCTCCAG |
| | | CACCTGGCCCAGCGAGACCGTCACCTGCAACGTTGCCCACCCGGCCAGCAGCACCAAGGTGG |
| | | ACAAGAAAATTGTGCCCAGGGATTGTCATCATCACCATCACCATCACTAATTTGACAGCTTTAAT |
| | | CATTCAATTAAGCTTTTAAT |
| 37 | VK_amino acid A1 | MKYLLPTAAAGLLLLAAQPAMADIVMSQSPSSMYASLGERVTITCKASQDINSYLNWFQQKPGKSPK |
| | | TLIYRANRLVDGVPSRFSGSGSGHDYFLTIRSLEYEDMGIYYCLQYDEFPYTFGGGTKLEIKRADAAP |
| | | TVSIFPPSSEQLTSGGASVVCFLNNFYPKDINVKWKIDGSERQNGVLNSWTDQDSKDSTYSMSSTL |
| | | TLTKDEYERHNSYTCEATHKTSTSPIVKSFNRNES |
| 38 | VK_amino acid A2 | MKYLLPTAAAGLLLLAAQPAMADIKMTQSPSSMYASLGERVTITCKASQDINSYLSWFQQKPGKSPK |
| | | TLIYRANRLVDGVPSRFSGSGSGQDYSLTISSLEYEDMGIYYCLQYDEFPYTFGGGTKLEIKRADAA |
| | | PTVSIFPPSSEQLTSGGASVVCFLNNFYPKDINVKWKIDGSERQNGVLNSWTDQDSKDSTYSMSST |
| | | LTLTKDEYERHNSYTCEATHKTSTSPIVKSFNRNES |
| 39 | VK_amino acid A3 | MKYLLPTAAAGLLLLAAQPAMADIQLTQSPSSMYASLGERVTIACKASQDINSYLSWFQQKPGKSPK |
| | | TLIHRANRLVDGVPSRFSGSGSGQDYSLTISSLEYEDIGIYYCLQYDEFPYTFGGGTKLEIKRADAAP |
| | | TVSIFPPSSEQLTSGGASVVCFLNNFYPKDINVKWKIDGSERQNGVLNSWTDQDSKDSTYSMSSTL |
| | | TLTKDEYERHNSYTCEATHKTSTSPIVKSFNRNES |
| 40 | VK_amino acid A4 | MKYLLPTAAAGLLLLAAQPAMADILLTQSPSSMYTSLGERVTITCKASQDINSYLSWFQQKPGKSPK |
| | | TLIYRANKLVDGVPSRFSGSGSGQDYSLTISSLESEDMGIYYCLQYDEFPYTFGGGTKLEIKRADAAP |
| | | TVSIFPPSSEQLTSGGASVVCFLNNFYPKDINVKWKIDGSERQNGVLNSWTDQDSKDSTYSMSSTL |
| | | TLTKDEYERHNSYTCEATHKTSTSPIVKSFNRNES |
| 41 | VK_amino acid A5 | MKYLLPTAAAGLLLLAAQPAMADIKMTQSPSSMYASLGERVTITCKASQDINSYLSWFQQKPGKSPK |
| | | TLIYRAKRLIDGVPSRFSGSGSGQDYSLTISSLEYEDMGIYYCLQYDEFPYTFGGGTKLEIKRADAAP |
| | | TVSIFPPSSEQLTSGGASVVCFLNNFYPKDINVKWKIDGSERQNGVLNSWTDQDSKDSTYSMSSTL |
| | | TLTKDEYERHNSYTCEATHKTSTSPIVKSFNRNES |
| 42 | VK_amino acid A6 | MKYLLPTAAAGLLLLAAQPAMADIVMSQSPSSMYASLGERVTITCKASQDINSYLSWFQQKPGKSPK |
| | | TLTYRANRLVEGVPSRFSGSGSGQDYSLTISSLEYEDMGIYYCLQYDEFPYTFGGGTKLEIKRADAA |
| | | PTVSIFPPSSEQLTSGGASVVCFLNNFYPKDINVKWKIDGSERQNGVLNSWTDQDSKDSTYSMSST |
| | | LTLTKDEYERHNSYTCEATHKTSTSPIVKSFNRNES |
| 43 | VK_amino acid A7 | MKYLLPTAAAGLLLLAAQPAMADIVMTQSPSSMYTSLGERVTITCKASQDINSYLSWFQQKPGKSPK |
| | | TLIYRANRLIDGVPSRFSGSGSGQDYSLTISSLEYEDMGIYYCLQYDEFPFTFGSGTKLEIKRADAAP |
| | | TVSIFPPSSEQLTSGGASVVCFLNNFYPKDINVKWKIDGSERQNGVLNSWTDQDSKDSTYSMSSTL |
| | | TLTKDEYERHNSYTCEATHKTSTSPIVKSFNRNES |
| 44 | VK_amino acid A8 | MKYLLPTAAAGLLLLAAQPAMANIVMTQSPVSLSMAIGEKVTIRCITNTDIDDAMNWYQQKPGEPPKL |
| | | LISEGNTLRPGVPSRFSSSGYGTDFVFTIENMLSEDVADYYCLQTDNLPLTFGSGTKLAIKRADAAPT |
| | | VSIFPPSSEQLTSGGASVVCFLNNFYPKDINVKWKIDGSERQNGVMNSWTDQDSKDSTYSMSSTLT |
| | | LTKDEYERHNSYTCEATHKTSTSPIVKSFNRNES |
| 45 | VK_amino acid A9 | MKYLLPTAAAGLLLLAAQPAMANIVMTQSPSSMYASLGERVTITCKASQDINSYLSWFQQKPGKSPK |
| | | TLIYRANRLVDGVPSRFSGSGSGQDYSLTISSLEYEDMGIYYCLQYDEFPYTFGGGTKLEIKRADAA |
| | | PTVSIFPPSSEQLTSGGASVVCFLNNFYPKDINVKWKIDGSERQNGVLNSWTDQDSKDSTYSMSST |
| | | LTLTKDEYERHNSYTCEATHKTSTSPIVKSFNRNES |
| 46 | VK_amino acid A10 | MKYLLPTAAAGLLLLAAQPAMANIVMTQSPSSMYASLGERVTITCKASQDIYSYLSWFQQKPGKSPK |
| | | TLIYRANRLVDGVPSRFSGSGSGQDYSLTISSLDYEDVGIYYCLQYDEFPYTFGSGTKLEIERADAAP |
| | | TVSIFPPSSEQLTSGGASVVCFLNNFYPKDINVKWKIDGSERQNGVLNSWTDQDSKDSTYSMSSTL |
| | | TLTKDEYERHNSYTCEATHKTSTSPIVKSFNRNES |
| 47 | VK_amino acid A11 | MKYLLPTAAAGLLLLAAQPAMAETTVTQSPVSLSMAIGEKVTIRCMTSTDIDDALNWYQQKPGEPPK |
| | | LLISEGNSLRPGVPSRFSSSGNGTDFVFTIENMLSEDVADYYCLQSDNLPLTFGSGTKLEIKRADAAP |
| | | TVSIFPPSSEQLTSGGASVVCFLNNFYPKDINVKWKIDGSERQNGVLNSWTDQDSKDSTYSMSSTL |
| | | TLTKDEYERHNSYTCEATHKTSTSPIVKSFNRNES |
| 48 | VK_amino acid A12 | MKYLLPTAAAGLLLLAAQPAMADIKMTQSPSSMYASLGERVTITCKASQDINSYLSWFQQKPGKSP |
| | | MTLTHRANRLVDGVPSRFSGSGSGQDYSLTISSLENEDMGIYYCLQYDEFPYTFGGGTKLEIKRADA |
| | | APTVSIFPPSSEQLTSGGASVVCFLNNFYPKDINVKWKIDGSERQNGVLNSWTDQDSKDSTYSMSS |
| | | TLTLTKDEYERHNSYTCEATHKTSTSPIVKSFNRNES |
| 49 | VK_amino acid A13 | MKYLLPTAAAGLLLLAAQPAMANIVMTQSPSSMYASLGERVTIICKSSQDINSYLSWFQQKPGKSPK |
| | | TLIFRANRLVDGVPSRFSGSGSGQDYSLTISSLEYEDMGIYYCLQYDEFPYTFGGGTKLEVKRADAA |
| | | PTVSIFPPSSEQLTSGGASVVCFLNNFYPKDINVKWKIDGSERQNGVLNSWTDQDSKDSTYSMSST |
| | | LTLTKDEYERHNSYTCEATHKTSTSPIVKSFNRNES |
| 50 | VK_amino acid A14 | MKYLLPTAAAGLLLLAAQPAMADVVMSQSPSSLAVSTGEKVTLSCKSSQSLLNSRTRKNYLAWYQQ |
| | | KPGQSPKLLIYWTSTRESGVPNRFTGSGSGTDFTLTISSVQAEDLAVYYCKQSYDLPWTFGGGTKL |
| | | EIKRADAAPTVSIFPPSSEQLTSGGASVVCFLNNFYPKDINVKWKIDGSERQNGVLNSWTDQDSKDS |
| | | TYSMSSTLTLTKDEYERHNSYTCEATHKTSTSPIVKSFNRNES |
| 51 | VK_nt A1 | TATCGCAACTCTCTACTGTTTCTCCATACCCGTTTTTTTGGATGGAGTGAAACGATGAAATACCT |
| | | ATTGCCTACGGCAGCCGCTGGATTGTTATTACTCGCTGCCCAACCAGCCATGGCCGACATCGT |
| | | TATGTCTCAGTCTCCATCTTCCATGTATGCATCTCTAGGAGAGAGAGTCACTATCACTTGCAAGG |
| | | CGAGTCAGGACATTAATAGCTATTTAAACTGGTTCCAGCAGAAACCAGGCAAATCTCCTAAGAC |
| | | CCTGATCTATCGTGCAAACAGATTGGTAGATGGGGTCCCATCAAGGTTCAGTGGCAGTGGATCT |
| | | GGGCACGATTATTTTCTTACCATTCGCAGCCTGGAATATGAAGATATGGGAATTTATTATTGTCT |
| | | ACAGTATGATGAGTTTCCGTACACGTTCGGAGGGGGGACCAAGCTGGAAATAAAACGGGCTGA |
| | | TGCTGCACCAACTGTATCCATCTTCCCACCATCCAGTGAGCAGTTAACATCTGGAGGTGCCTCA |
| | | GTCGTGTGCTTCTTGAACAACTTCTACCCCAAAGACATCAATGTCAAGTGGAAGATTGATGGCA |
| | | GTGAACGACAAAATGGCGTCCTGAACAGTTGGACTGATCAGGACAGCAAAGACAGCACCTACA |
| | | GCATGAGCAGCACCCTCACGTTGACCAAGGACGAGTATGAACGACATAACAGCTATACCTGTG |
| | | AGGCCACTCACAAGACATCAACTTCACCCATTGTCAAGAGCTTCAACAGGAATGAGTCTTAAGT |
| | | GATTAGCTAATTCTAGAACGCGTCACTTGGCACTGGCCGTCG |
| 52 | VK_nt A2 | TTATCGCAACTCTCTACTGTTTCTCCATACCCGTTTTTTTGGATGGAGTGAAACGATGAAATACC |
| | | TATTGCCTACGGCAGCCGCTGGATTGTTATTACTCGCTGCCCAACCAGCCATGGCCGACATCAA |
| | | AATGACCCAGTCTCCATCTTCCATGTATGCATCTCTAGGAGAGAGAGTCACTATCACTTGCAAG |
| | | GCGAGTCAGGACATTAATAGCTATTTAAGCTGGTTCCAGCAGAAACCAGGGAAATCTCCTAAGA |
| | | CCCTGATCTATCGTGCAAACAGATTGGTAGATGGGGTCCCATCAAGGTTCAGTGGCAGTGGAT |
| | | CTGGGCAAGATTATTCTCTCACCATCAGCAGCCTGGAGTATGAAGATATGGGAATTTATTATTGT |
| | | CTACAGTATGATGAATTTCCGTACACGTTCGGAGGGGGGACCAAGCTGGAAATAAAACGGGCT |
| | | GATGCTGCACCAACTGTATCCATCTTCCCACCATCCAGTGAGCAGTTAACATCTGGAGGTGCCT |
| | | CAGTCGTGTGCTTCTTGAACAACTTCTACCCCAAAGACATCAATGTCAAGTGGAAGATTGATGG |
| | | CAGTGAACGACAAAATGGCGTCCTGAACAGTTGGACTGATCAGGACAGCAAAGACAGCACCTA |
| | | CAGCATGAGCAGCACCCTCACGTTGACCAAGGACGAGTATGAACGACATAACAGCTATACCTG |
| | | TGAGGCCACTCACAAGACATCAACTTCACCCATTGTCAAGAGCTTCAACAGGAATGAGTCTTAA |
| | | GTGATTAGCTAATTCTAGAACGCGTCACTTGGCACTGGCCGTCGTTTTACAACGTCGTGACTGG |
| | | GAAAA |
| 53 | VK_nt A3 | TATCGCAACTCTCTACTGTTTCTCCATACCCGTTTTTTTGGATGGAGTGAAACGATGAAATACCT |
| | | ATTGCCTACGGCAGCCGCTGGATTGTTATTACTCGCTGCCCAACCAGCCATGGCCGACATTCA |
| | | GCTGACCCAGTCTCCATCTTCCATGTATGCATCTCTAGGAGAGAGAGTCACTATCGCTTGCAAG |
| | | GCGAGTCAGGACATTAATAGCTATTTAAGCTGGTTCCAGCAGAAACCAGGGAAATCTCCTAAGA |
| | | CCCTGATCCATCGTGCAAACAGATTGGTAGATGGGGTCCCATCAAGGTTCAGTGGCAGTGGAT |
| | | CTGGGCAAGATTATTCTCTCACCATCAGCAGCCTGGAGTATGAAGATATCGGAATTTATTATTGT |
| | | CTACAGTATGATGAGTTTCCGTACACGTTCGGAGGGGGGACCAAGCTGGAAATAAAACGGGCT |
| | | GATGCTGCACCAACTGTATCCATCTTCCCACCATCCAGTGAGCAGTTAACATCTGGAGGTGCCT |
| | | CAGTCGTGTGCTTCTTGAACAACTTCTACCCCAAAGACATCAATGTCAAGTGGAAGATTGATGG |
| | | CAGTGAACGACAAAATGGCGTCCTGAACAGTTGGACTGATCAGGACAGCAAAGACAGCACCTA |
| | | CAGCATGAGCAGCACCCTCACGTTGACCAAGGACGAGTATGAACGACATAACAGCTATACCTG |
| | | TGAGGCCACTCACAAGACATCAACTTCACCCATTGTCAAGAGCTTCAACAGGAATGAGTCTTAA |
| | | GTGATTAGCTAATTCTAGAACGCGTCACTTGGCACTGGCCGTCGTTTTACAACGTCG |
| 54 | VK_nt A4 | TCGCAACTCTCTACTGTTTCTCCATACCCGTTTTTTTGGATGGAGTGAAACGATGAAATACCTAT |
| | | TGCCTACGGCAGCCGCTGGATTGTTATTACTCGCTGCCCAACCAGCCATGGCCGACATCTTGC |
| | | TGACTCAGTCTCCATCTTCCATGTATACATCTCTAGGAGAGAGAGTCACTATCACTTGCAAGGC |
| | | GAGTCAGGACATTAATAGCTATTTAAGCTGGTTCCAGCAGAAACCAGGAAAATCTCCTAAGACC |
| | | CTGATCTATCGTGCAAACAAATTGGTAGATGGGGTCCCATCAAGATTCAGTGGCAGTGGATCTG |
| | | GGCAAGATTATTCTCTCACCATCAGCAGCCTGGAGTCTGAAGATATGGGAATTTATTATTGTCTA |
| | | CAGTATGATGAGTTTCCGTACACGTTCGGAGGGGGGACCAAGCTGGAAATCAAACGGGCTGAT |
| | | GCTGCACCAACTGTATCCATCTTCCCACCATCCAGTGAGCAGTTAACATCTGGAGGTGCCTCAG |
| | | TCGTGTGCTTCTTGAACAACTTCTACCCCAAAGACATCAATGTCAAGTGGAAGATTGATGGCAG |
| | | TGAACGACAAAATGGCGTCCTGAACAGTTGGACTGATCAGGACAGCAAAGACAGCACCTACAG |
| | | CATGAGCAGCACCCTCACGTTGACCAAGGACGAGTATGAACGACATAACAGCTATACCTGTGA |
| | | GGCCACTCACAAGACATCAACTTCACCCATTGTCAAGAGCTTCAACAGGAATGAGTCTTAAGTG |
| | | ATTAGCTAATTCTAGAACGCGTCACTTGG |
| 55 | VK_nt A5 | TCGCAACTCTCTACTGTTTCTCCATACCCGTTTTTTTGGATGGAGTGAAACGATGAAATACCTAT |
| | | TGCCTACGGCAGCCGCTGGATTGTTATTACTCGCTGCCCAACCAGCCATGGCCGACATCAAAA |
| | | TGACCCAGTCTCCATCTTCCATGTATGCATCTCTAGGAGAGAGAGTCACTATCACTTGCAAGGC |
| | | GAGTCAGGACATTAATAGCTATTTAAGCTGGTTCCAGCAGAAACCAGGGAAATCTCCTAAGACC |
| | | CTGATCTATCGTGCAAAGAGATTGATAGATGGGGTCCCATCAAGGTTCAGTGGCAGTGGATCTG |
| | | GGCAAGATTATTCTCTCACCATCAGCAGCCTGGAGTATGAAGATATGGGAATTTATTATTGTCTA |
| | | CAGTATGATGAGTTTCCTTACACGTTCGGAGGGGGGACAAAGTTGGAAATAAAACGGGCTGAT |
| | | GCTGCACCAACTGTATCCATCTTCCCACCATCCAGTGAGCAGTTAACATCTGGAGGTGCCTCAG |
| | | TCGTGTGCTTCTTGAACAACTTCTACCCCAAAGACATCAATGTCAAGTGGAAGATTGATGGCAG |
| | | TGAACGACAAAATGGCGTCCTGAACAGTTGGACTGATCAGGACAGCAAAGACAGCACCTACAG |
| | | CATGAGCAGCACCCTCACGTTGACCAAGGACGAGTATGAACGACATAACAGCTATACCTGTGA |
| | | GGCCACTCACAAGACATCAACTTCACCCATTGTCAAGAGCTTCAACAGGAATGAGTCTTAAGTG |
| | | ATTAGCTAATTCTAGAACGCGTCACTTGGCACTGGCCGTCGTTTTACAACGTCG |
| 56 | VK_nt A6 | TTTTATCGCAACTCTCTACTGTTTCTCCATACCCGTTTTTTTGGATGGAGTGAAACGATGAAATA |
| | | CCTATTGCCTACGGCAGCCGCTGGATTGTTATTACTCGCTGCCCAACCAGCCATGGCCGACAT |
| | | CGTTATGTCTCAGTCTCCATCTTCCATGTATGCATCTCTAGGAGAGAGAGTCACTATCACTTGCA |
| | | AGGCGAGTCAAGACATTAATAGCTATTTAAGCTGGTTCCAGCAGAAACCAGGGAAATCTCCTAA |
| | | GACCCTGACCTATCGTGCAAACAGATTGGTAGAAGGGGTCCCATCAAGGTTCAGTGGCAGTGG |
| | | ATCTGGGCAAGATTATTCTCTCACCATCAGCAGCCTGGAATATGAAGATATGGGAATTTATTATT |
| | | GTCTACAGTATGATGAGTTTCCGTACACGTTCGGAGGGGGGACCAAGCTGGAAATAAAACGGG |
| | | CTGATGCTGCACCAACTGTATCCATCTTCCCACCATCCAGTGAGCAGTTAACATCTGGAGGTGC |
| | | CTCAGTCGTGTGCTTCTTGAACAACTTCTACCCCAAAGACATCAATGTCAAGTGGAAGATTGAT |
| | | GGCAGTGAACGACAAAATGGCGTCCTGAACAGTTGGACTGATCAGGACAGCAAAGACAGCACC |
| | | TACAGCATGAGCAGCACCCTCACGTTGACCAAGGACGAGTATGAACGACATAACAGCTATACCT |
| | | GTGAGGCCACTCACAAGACATCAACTTCACCCATTGTCAAGAGCTTCAACAGGAATGAGTCTTA |
| | | AGTGATTAGCTAATTCTAGAACGCGTCACTTGGCACTGGCCGTCGTTTTACAACGT |
| 57 | VK_nt A7 | GCAACTCTCTACTGTTTCTCCATACCCGTTTTTTTGGATGGAGTGAAACGATGAAATACCTATTG |
| | | CCTACGGCAGCCGCTGGATTGTTATTACTCGCTGCCCAACCAGCCATGGCCGACATTGTGATG |
| | | ACCCAGTCTCCATCTTCCATGTATACATCTCTAGGAGAGAGAGTCACTATCACTTGCAAGGCGA |
| | | GTCAGGACATTAATAGCTATTTAAGCTGGTTCCAGCAGAAACCAGGGAAATCTCCTAAGACCCT |
| | | GATCTATCGTGCAAACAGATTGATAGATGGGGTCCCATCAAGGTTCAGTGGCAGTGGATCTGG |
| | | GCAAGATTATTCTCTCACCATCAGCAGCCTGGAGTATGAAGATATGGGAATTTATTATTGTCTAC |
| | | AGTATGATGAGTTTCCATTCACGTTCGGCTCGGGGACAAAGTTGGAAATAAAACGGGCTGATGC |
| | | TGCACCAACTGTATCCATCTTCCCACCATCCAGTGAGCAGTTAACATCTGGAGGTGCCTCAGTC |
| | | GTGTGCTTCTTGAACAACTTCTACCCCAAAGACATCAATGTCAAGTGGAAGATTGATGGCAGTG |
| | | AACGACAAAATGGCGTCCTGAACAGTTGGACTGATCAGGACAGCAAAGACAGCACCTACAGCA |
| | | TGAGCAGCACCCTCACGTTGACCAAGGACGAGTATGAACGACATAACAGCTATACCTGTGAGG |
| | | CCACTCACAAGACATCAACTTCACCCATTGTCAAGAGCTTCAACAGGAATGAGTCTTAAGTGATT |
| | | AGCTAATTCTAGAACGCGTCACTTGGCACTGGCCGTCGTTTTACAACGTCGTGACTGGGAAAAC |
| | | CCTGGC |
| 58 | VK_nt A8 | GAGTGAAACGATGAAATACCTATTGCCTACGGCAGCCGCTGGATTGTTATTACTCGCTGCCCAA |
| | | CCAGCCATGGCCAACATCGTTATGACCCAGTCTCCAGTATCCCTGTCCATGGCTATAGGAGAAA |
| | | AAGTCACCATCAGATGCATAACCAACACTGATATTGATGATGCTATGAACTGGTACCAGCAAAA |
| | | GCCAGGGGAACCTCCTAAGCTCCTTATTTCAGAAGGCAATACTCTTCGTCCTGGAGTCCCATCC |
| | | CGATTCTCCAGCAGTGGCTATGGTACAGATTTTGTTTTTACAATTGAAAACATGCTCTCAGAAGA |
| | | TGTTGCAGATTACTACTGTTTGCAAACTGATAACTTGCCTCTCACGTTCGGCTCGGGGACAAAG |
| | | TTGGCAATAAAACGGGCTGATGCTGCACCAACTGTATCCATCTTCCCACCATCCAGTGAGCAGT |
| | | TAACATCTGGAGGTGCCTCAGTCGTGTGCTTCTTGAACAACTTCTACCCCAAAGACATCAATGT |
| | | CAAGTGGAAGATTGATGGCAGTGAACGACAAAATGGCGTCATGAACAGTTGGACTGATCAGGA |
| | | CAGCAAAGACAGCACCTACAGCATGAGCAGCACCCTCACGTTGACCAAGGACGAGTATGAACG |
| | | ACATAACAGCTATACCTGTGAGGCCACTCACAAGACATCAACTTCACCCATTGTCAAGAGCTTC |
| | | AACAGGAATGAGTCTTAAGTGATTAG |
| 59 | VK_nt A9 | TCGCAACTCTCTACTGTTTCTCCATACCCGTTTTTTTGGATGGAGTGAAACGATGAAATACCTAT |
| | | TGCCTACGGCAGCCGCTGGATTGTTATTACTCGCTGCCCAACCAGCCATGGCCAACATCGTTAT |
| | | GACCCAGTCTCCATCTTCCATGTATGCATCTCTAGGAGAGAGAGTCACTATCACTTGCAAGGCG |
| | | AGTCAGGACATTAATAGCTATTTAAGCTGGTTCCAGCAGAAACCAGGGAAATCTCCTAAGACCC |
| | | TGATCTATCGTGCAAACAGATTGGTAGATGGGGTCCCATCAAGGTTCAGTGGCAGTGGATCTG |
| | | GGCAAGATTATTCTCTCACCATCAGCAGCCTGGAGTATGAAGATATGGGAATTTATTATTGTCTA |
| | | CAGTATGATGAGTTTCCGTACACGTTCGGAGGGGGGACCAAACTGGAAATAAAACGGGCTGAT |
| | | GCTGCACCAACTGTATCCATCTTCCCACCATCCAGTGAGCAGTTAACATCTGGAGGTGCCTCAG |
| | | TCGTGTGCTTCTTGAACAACTTCTACCCCAAAGACATCAATGTCAAGTGGAAGATTGATGGCAG |
| | | TGAACGACAAAATGGCGTCCTGAACAGTTGGACTGATCAGGACAGCAAAGACAGCACCTACAG |
| | | CATGAGCAGCACCCTCACGTTGACCAAGGACGAGTATGAACGACATAACAGCTATACCTGTGA |
| | | GGCCACTCACAAGACATCAACTTCACCCATTGTCAAGAGCTTCAACAGGAATGAGTCTTAAGTG |
| | | ATTAGCTAATTCTAGAACGCGTCACTTGGCACTGGCCGTCGTTTTACA |
| 60 | VK_nt A10 | GCAACTCTCTACTGTTTCTCCATACCCGTTTTTTTGGATGGAGTGAAACGATGAAATACCTATTG |
| | | CCTACGGCAGCCGCTGGATTGTTATTACTCGCTGCCCAACCAGCCATGGCCAACATCGTTATGA |
| | | CCCAGTCTCCATCTTCCATGTATGCATCTCTAGGAGAGAGGGTCACTATCACTTGCAAGGCGAG |
| | | TCAGGACATTTATAGCTATTTAAGCTGGTTCCAGCAGAAACCAGGCAAATCTCCTAAGACCCTG |
| | | ATCTATCGTGCAAACAGATTGGTAGATGGGGTCCCATCAAGGTTCAGTGGCAGTGGATCTGGG |
| | | CAAGATTATTCTCTCACCATCAGCAGCCTGGACTATGAAGATGTGGGAATTTATTATTGTCTACA |
| | | GTATGATGAGTTTCCGTACACGTTCGGCTCGGGGACAAAGTTGGAAATAGAACGGGCTGATGC |
| | | TGCACCAACTGTATCCATCTTCCCACCATCCAGTGAGCAGTTAACATCTGGAGGTGCCTCAGTC |
| | | GTGTGCTTCTTGAACAACTTCTACCCCAAAGACATCAATGTCAAGTGGAAGATTGATGGCAGTG |
| | | AACGACAAAATGGCGTCCTGAACAGTTGGACTGATCAGGACAGCAAAGACAGCACCTACAGCA |
| | | TGAGCAGCACCCTCACGTTGACCAAGGACGAGTATGAACGACATAACAGCTATACCTGTGAGG |
| | | CCACTCACAAGACATCAACTTCACCCATTGTCAAGAGCTTCAACAGGAATGAGTCTTAAGTGATT |
| | | AGCTAATTCTAGAACGCGTCACTTGGCACTGGCCGTCGTTTTA |
| 61 | VK_nt A11 | CGCAACTCTCTACTGTTTCTCCATACCCGTTTTTTTGGATGGAGTGAAACGATGAAATACCTATT |
| | | GCCTACGGCAGCCGCTGGATTGTTATTACTCGCTGCCCAACCAGCCATGGCCGAAACAACTGT |
| | | GACCCAGTCTCCAGTATCCCTGTCCATGGCTATAGGAGAAAAAGTCACCATCAGATGCATGACC |
| | | AGCACTGATATTGATGATGCTCTGAACTGGTACCAGCAAAAGCCAGGGGAACCTCCTAAACTCC |
| | | TTATTTCAGAAGGCAATAGTCTTCGTCCTGGAGTCCCATCCCGATTCTCCAGCAGTGGCAATGG |
| | | TACAGATTTTGTTTTTACAATTGAAAACATGCTCTCAGAAGATGTTGCAGATTACTACTGTTTGCA |
| | | AAGTGATAACTTGCCTCTCACGTTCGGCTCGGGGACAAAGTTGGAAATAAAACGGGCTGATGCT |
| | | GCACCAACTGTATCCATCTTCCCACCATCCAGTGAGCAGTTAACATCTGGAGGTGCCTCAGTCG |
| | | TGTGCTTCTTGAACAACTTCTACCCCAAAGACATCAATGTCAAGTGGAAGATTGATGGCAGTGA |
| | | ACGACAAAATGGCGTCCTGAACAGTTGGACTGATCAGGACAGCAAAGACAGCACCTACAGCAT |
| | | GAGCAGCACCCTCACGTTGACCAAGGACGAGTATGAACGACATAACAGCTATACCTGTGAGGC |
| | | CACTCACAAGACATCAACTTCACCCATTGTCAAGAGCTTCAACAGGAATGAGTCTTAAGTGATTA |
| | | GCTAATTCTAGAATGCGTCACTTGGCACTGGCCGTCGTTTTACAACGTCGTGAC |
| 62 | VK_nt A12 | TATCGCAACTCTCTACTGTTTCTCCATACCCGTTTTTTTGGATGGAGTGAAACGATGAAATACCT |
| | | ATTGCCTACGGCAGCCGCTGGATTGTTATTACTCGCTGCCCAACCAGCCATGGCCGACATCAA |
| | | AATGACCCAGTCTCCATCTTCCATGTATGCATCTCTAGGAGAGAGAGTCACTATCACTTGCAAG |
| | | GCGAGTCAGGACATTAATAGCTATTTAAGCTGGTTCCAGCAGAAACCAGGGAAATCTCCTATGA |
| | | CCCTGACCCATCGTGCAAACAGATTGGTAGATGGGGTCCCATCAAGGTTCAGTGGCAGTGGAT |
| | | CTGGGCAAGATTATTCTCTCACCATCAGCAGCCTGGAGAATGAAGATATGGGAATTTATTATTGT |
| | | CTACAGTATGATGAGTTTCCGTACACGTTCGGAGGGGGGACCAAGCTGGAAATAAAACGGGCT |
| | | GATGCTGCACCAACTGTATCCATCTTCCCACCATCCAGTGAGCAGTTAACATCTGGAGGTGCCT |
| | | CAGTCGTGTGCTTCTTGAACAACTTCTACCCCAAAGACATCAATGTCAAGTGGAAGATTGATGG |
| | | CAGTGAACGACAAAATGGCGTCCTGAACAGTTGGACTGATCAGGACAGCAAAGACAGCACCTA |
| | | CAGCATGAGCAGCACCCTCACGTTGACCAAGGACGAGTATGAACGACATAACAGCTATACCTG |
| | | TGAGGCCACTCACAAGACATCAACTTCACCCATTGTCAAGAGCTTCAACAGGAATGAGTCTTAA |
| | | GTGATTAGCTAATTCTAGAACGCGTCACTTGGCACTGGCCGTCGTTTTACAACGTCGT |
| 63 | VK_nt A13 | CGCAACTCTCTACTGTTTCTCCATACCCGTTTTTTTGGATGGAGTGAAACGATGAAATACCTATT |
| | | GCCTACGGCAGCCGCTGGATTGTTATTACTCGCTGCCCAACCAGCCATGGCCAACATCGTTAT |
| | | GACCCAGTCTCCATCTTCCATGTATGCATCTCTAGGAGAGAGAGTCACTATCATTTGCAAGTCG |
| | | AGTCAGGACATTAATAGCTATTTAAGTTGGTTCCAGCAGAAACCAGGGAAGTCTCCTAAGACCC |
| | | TGATCTTTCGTGCAAACAGATTGGTAGATGGGGTCCCATCAAGGTTCAGTGGCAGTGGATCTG |
| | | GGCAAGATTATTCTCTCACCATCAGCAGCCTGGAGTATGAAGATATGGGAATTTATTATTGTCTA |
| | | CAGTATGATGAGTTTCCGTACACGTTCGGAGGGGGGACCAAGCTGGAAGTAAAACGGGCTGAT |
| | | GCTGCACCAACCGTATCCATCTTCCCACCATCCAGTGAGCAGTTAACATCTGGAGGTGCCTCAG |
| | | TCGTGTGCTTCTTGAACAACTTCTACCCCAAAGACATCAATGTCAAGTGGAAGATTGATGGCAG |
| | | TGAACGACAAAATGGCGTCCTGAACAGTTGGACTGATCAGGACAGCAAAGACAGCACCTACAG |
| | | CATGAGCAGCACCCTCACGTTGACCAAGGACGAGTATGAACGACATAACAGCTATACCTGTGA |
| | | GGCCACTCACAAGACATCAACTTCACCCATTGTCAAGAGCTTCAACAGGAATGAGTCTTAAGTG |
| | | ATTAGCTAATTCTAGAACGCGTCACTTGGCACTGGCCGTCGTTTTACAACGTCGTGA |
| 64 | VK_nt A14 | TCGCAACTCTCTACTGTTTCTCCATACCCGTTTTTTTGGATGGAGTGAAACGATGAAATACCTAT |
| | | TGCCTACGGCAGCCGCTGGATTGTTATTACTCGCTGCCCAACCAGCCATGGCCGACGTTGTGA |
| | | TGTCACAGTCTCCATCCTCCCTGGCTGTGTCAACAGGAGAGAAGGTCACTTTGAGCTGCAAATC |
| | | CAGTCAGAGTCTGCTCAACAGTAGAACCCGAAAGAACTACTTGGCTTGGTACCAGCAGAAACCA |
| | | GGGCAGTCTCCTAAACTGCTGATCTACTGGACATCCACTAGGGAATCTGGGGTCCCTAATCGCT |
| | | TCACAGGCAGTGGATCTGGGACAGATTTCACTCTCACCATCAGCAGTGTGCAGGCTGAAGACC |
| | | TGGCAGTTTATTACTGCAAGCAATCTTATGATCTTCCGTGGACGTTCGGTGGGGGCACCAAACT |
| | | GGAAATCAAACGGGCTGATGCTGCACCAACTGTATCCATCTTCCCACCATCCAGTGAGCAGTTA |
| | | ACATCTGGAGGTGCCTCAGTCGTGTGCTTCTTGAACAACTTCTACCCCAAAGACATCAATGTCA |
| | | AGTGGAAGATTGATGGCAGTGAACGACAAAATGGCGTCCTGAACAGTTGGACTGATCAGGACA |
| | | GCAAAGACAGCACCTACAGCATGAGCAGCACCCTCACGTTGACCAAGGACGAGTATGAACGAC |
| | | ATAACAGCTATACCTGTGAGGCCACTCACAAGACATCAACTTCACCCATTGTCAAGAGCTTCAA |
| | | CAGGAATGAGTCTTAAGTGATTAGCTAATTCTAGAACGCGTCACTTGGCACTGGCCGTCGT |

Other embodiments of the invention which may be mentioned include:

1. A method for treating or preventing bladder cancer, breast cancer, colorectal cancer, head and neck cancer, liver cancer, lung cancer, ovarian cancer, pancreatic cancer, skin cancer or thyroid cancer which comprises administering to a subject in need thereof a therapeutically effective amount of a composition comprising an affinity reagent capable of specific binding to ROR1 or a fragment thereof, and a pharmaceutically acceptable diluent or carrier, wherein ROR1 is overexpressed in said cancers.

2. An affinity reagent capable of specific binding to ROR1 or a fragment thereof.

3. An affinity reagent according to 2 which contains or is conjugated to a therapeutic moiety.

4. An affinity reagent according to 3 wherein the therapeutic moiety is a cytotoxic moiety or a radioactive isotype.

5. An affinity reagent according to 2 which contains or is conjugated to a detectable label.

6. An affinity reagent according to any one of 2 to 5 which is an antibody.

7. An antibody according to 6 which is an isolated monoclonal antibody, or an antigen-binding portion thereof, an antibody fragment, or an antibody mimetic.

8. An isolated monoclonal antibody according to 7 wherein said antibody is a full-length antibody of an IgG1, IgG2, IgG3, or IgG4 isotype.

9. An isolated monoclonal antibody according to 7 wherein said antibody is selected from the group consisting of: a whole antibody, an antibody fragment, a humanised antibody, a single chain antibody, an immunoconjugate, a defucosylated antibody, and a bispecific antibody.

10. An antibody fragment according to 7, wherein the fragment is selected from the group consisting of: a UniBody, a domain antibody and a Nanobody.

11. An antibody mimetic according to 7, wherein the mimetic is selected from the group consisting of: an Affibody, a DARPin, an Anticalin, an Avimer, a Versabody, and a Duocalin.

12. A monoclonal antibody according to 7, which has cytotoxicity against ROR1 antigen expressing cells in the presence of a human complement.

13. A monoclonal antibody according to 7, which has cytotoxicity against ROR1 antigen expressing cells in the presence of human immune effector cells.

14. A pharmaceutical composition comprising a therapeutically effective amount of an affinity reagent or a fragment thereof as defined in any one of 2 to 13, and a pharmaceutically acceptable diluent or carrier.

15. A pharmaceutical composition according to 14 comprising one or more affinity reagents as defined in any one of 2 to 13 and a pharmaceutically acceptable excipient.

16. An agent as defined in any one of 2 to 13 or a composition as defined in 14 or 15 for use in treating or preventing disease.

17. An agent according to 16 wherein the disease is cancer.

18. An agent according to claim 17 wherein the cancer is bladder cancer, breast cancer, colorectal cancer, head and neck cancer, liver cancer, lung cancer, ovarian cancer, pancreatic cancer, skin cancer or thyroid cancer.

19. ROR1, or a fragment thereof for use in treating or preventing disease.

20. ROR1, or a fragment thereof according to 19 wherein the disease is cancer.

21. ROR1, or a fragment thereof according to 20 wherein the cancer is bladder cancer, breast cancer, colorectal cancer, head and neck cancer, liver cancer, lung cancer, ovarian cancer, pancreatic cancer, skin cancer or thyroid cancer.

22. A method for treating or preventing bladder cancer, breast cancer, colorectal cancer, head and neck cancer, liver cancer, lung cancer, ovarian cancer, pancreatic cancer, skin cancer or thyroid cancer which comprises administering to a subject in need thereof a therapeutically effective amount of an agent as defined in any one of 2 to 13 or a composition as defined in 14 or 15.

23. An isolated nucleic acid molecule encoding the isolated antibody or antigen-binding portion thereof of 7.

24. An expression vector comprising the nucleic acid molecule of 23.

25. A host cell comprising the expression vector of 24.

26. A kit containing one or more affinity reagents according to any one of 2 to 13 or a composition as defined in 14, wherein said affinity reagent is suitable for use in treatment and/or diagnosis.

27. A kit according to 26, which further comprises instructions for use of said affinity reagent as defined in any one of 16 to 18.

28. A kit according to 26 or 27 which further comprises a hybridising agent.

29. A method of screening for compounds that modulate the activity of ROR1, the method comprising: (a) contacting ROR1 or a biologically active portion thereof with a candidate compound; and (b) determining whether activity of ROR1 is thereby modulated.

30. A method according to 29 which comprises (a) contacting ROR1 or a biologically active portion thereof with a candidate compound in a sample; and (b) comparing the activity of ROR1 or a biologically active portion thereof in said sample after contact with said candidate compound with the activity of ROR1 or a biologically active portion thereof in said sample before contact with said candidate compound, or with a reference level of activity.

31. A method according to 29 or 30 which is a method of screening for compounds that inhibit activity of ROR1.

32. A method according to any one of 29 to 31 wherein ROR1 or a biologically active portion thereof is expressed on or by a cell.

33. A method according to any one of 29 to 31 wherein ROR1 or a biologically active portion thereof is isolated from cells which express it.

34. A method according to 33 wherein ROR1 or a biologically active portion thereof is immobilised onto a solid phase.

35. A method of screening for compounds that modulate the expression of ROR1 or nucleic acid encoding ROR1, the method comprising: (a) contacting cells expressing ROR1 or nucleic acid encoding ROR1 with a candidate compound; and (b) determining whether expression of ROR1 or nucleic acid encoding ROR1 is thereby modulated.

36. A method according to 35 which comprises (a) contacting cells expressing ROR1 or nucleic acid encoding ROR1 with a candidate compound in a sample; and (b) comparing the expression of ROR1 or nucleic acid encoding ROR1 by cells in said sample after contact with said candidate compound with the expression of ROR1 or nucleic acid encoding ROR1 of cells in said sample before contact with said candidate compound, or with a reference level of expression.

37. A method according to 35 or 36 which is a method of screening for compounds that inhibit expression of ROR1 or nucleic acid encoding ROR1.

38. A compound obtainable by a method according to any one of 29 to 37.

39. A compound which modulates the activity or expression of ROR1 or nucleic acid encoding ROR1.

40. A compound according to 39 which inhibits the activity or expression of ROR1 or nucleic acid encoding ROR1.

41. A compound according to any one of 38 to 40 for use in treating or preventing bladder cancer, breast cancer, colorectal cancer, head and neck cancer, liver cancer, lung cancer, ovarian cancer, pancreatic cancer, skin cancer or thyroid cancer.

42. A method for treating or preventing bladder cancer, breast cancer, colorectal cancer, head and neck cancer, liver cancer, lung cancer, ovarian cancer, pancreatic cancer, skin cancer or thyroid cancer which comprises administering to a subject in need thereof a therapeutically effective amount of a compound according to any one of 38 to 40.

43. A hybridizing agent capable of hybridizing to nucleic acid encoding ROR1 and inhibiting transcription of mRNA.

44. A hybridizing agent according to 43 which contains or is conjugated to a detectable label.

45. A pharmaceutical composition comprising one or more hybridizing agents as defined in 43 or 44 and a pharmaceutically acceptable diluent or carrier.

46. A kit containing one or more hybridizing agents according to any one of 43 to 45 wherein said hybridising agent is suitable for use in treatment and/or diagnosis.

47. A kit according to 46 further containing reagents capable of detecting and reporting the binding of said hybridizing agents to their binding partners.

48. A hybridizing agent as defined in any one of 43 or 44 for use in treatment.

49. A hybridizing agent according to 48 wherein the treatment is for cancer.

50. A hybridizing agent according to 49, wherein the cancer is selected from bladder cancer, breast cancer, colorectal cancer, head and neck cancer, liver cancer, lung cancer, ovarian cancer, pancreatic cancer, skin cancer or thyroid cancer.

51. A method for treating or preventing bladder cancer, breast cancer, colorectal cancer, head and neck cancer, liver cancer, lung cancer, ovarian cancer, pancreatic cancer, skin cancer or thyroid cancer which comprises administering to a subject in need thereof a therapeutically effective amount of a composition comprising a hybridizing agent capable of hybridizing to nucleic acid encoding ROR1, and a pharmaceutically acceptable diluent or carrier.

52. An immunogenic composition comprising ROR1 or an epitope containing fragment thereof, or nucleic acid encoding ROR1 or a fragment thereof optionally together with an immunostimulant.

53. A vaccine composition comprising ROR1 or an epitope containing fragment thereof, or nucleic acid encoding ROR1 or an epitope containing fragment thereof optionally together with an immunostimulant.

54. A method of raising an immune response which comprises administering to a subject a composition according to 52.

55. A method for treating or preventing bladder cancer, breast cancer, colorectal cancer, head and neck cancer, liver cancer, lung cancer, ovarian cancer, pancreatic cancer, skin cancer or thyroid cancer which comprises administering to a subject in need thereof a therapeutically effective amount of a composition according to 52 or 53.

56. A composition according to 52 or 53 for use in preventing or treating bladder cancer, breast cancer, colorectal cancer, head and neck cancer, liver cancer, lung cancer, ovarian cancer, pancreatic cancer, skin cancer or thyroid cancer.

57. A method of detecting, diagnosing and/or screening for or monitoring the progression of bladder cancer, breast cancer, colorectal cancer, head and neck cancer, liver cancer, lung cancer, ovarian cancer, pancreatic cancer, skin cancer or thyroid cancer or of monitoring the effect of an anti-bladder cancer, anti-breast cancer, anti-colorectal cancer, anti-head and neck cancer, anti-liver cancer, anti-lung cancer, anti-ovarian cancer, anti-pancreatic cancer, anti-skin cancer or anti-thyroid cancer drug or therapy in a subject which comprises detecting the presence or level of ROR1, or one or more fragments thereof, or the presence or level of nucleic acid encoding ROR1 or the presence or level of the activity of ROR1 or which comprises detecting a change in the level thereof in said subject.

58. A method of detecting, diagnosing and/or screening for bladder cancer, breast cancer, colorectal cancer, head and neck cancer, liver cancer, lung cancer, ovarian cancer, pancreatic cancer, skin cancer or thyroid cancer in a candidate subject which comprises detecting the presence of ROR1, or one or more fragments thereof, or the presence of nucleic acid encoding ROR1 or the presence of the activity of ROR1 in said candidate subject, in which either (a) the presence of an elevated level of ROR1 or said one or more fragments thereof or an elevated level of nucleic acid encoding ROR1 or the presence of an elevated level of ROR1 activity in the candidate subject as compared with the level in a healthy subject or (b) the presence of a detectable level of ROR1 or said one or more fragments thereof or a detectable level of nucleic acid encoding ROR1 or the presence of a detectable level of ROR1 activity in the candidate subject as compared with a corresponding undetectable level in a healthy subject indicates the presence of bladder cancer, breast cancer, colorectal cancer, head and neck cancer, liver cancer, lung cancer, ovarian cancer, pancreatic cancer, skin cancer or thyroid cancer in said subject.

59. A method of monitoring the progression of bladder cancer, breast cancer, colorectal cancer, head and neck cancer, liver cancer, lung cancer, ovarian cancer, pancreatic cancer, skin cancer or thyroid cancer in a subject or of monitoring the effect of an anti-bladder cancer, anti-breast cancer, anti-colorectal cancer, anti-head and neck cancer, anti-liver cancer, anti-lung cancer, anti-ovarian cancer, anti-pancreatic cancer, anti-skin cancer or anti-thyroid cancer drug or therapy which comprises detecting the presence of ROR1, or one or more fragments thereof, or the presence of nucleic acid encoding ROR1 or the presence of the activity of ROR1 in said candidate subject at a first time point and at a later time point, the presence of an elevated or lowered level of ROR1 or said one or more fragments thereof or an elevated or lowered level of nucleic acid encoding ROR1 or the presence of an elevated or lowered level of ROR1 activity in the subject at the later time point as compared with the level in the subject at said first time point, indicating the progression or regression of said cancer or indicating the effect or non-effect of said anti-cancer drug or therapy in said subject.

60. A method according to any one of 57 to 59 wherein the presence of ROR1, or one or more fragments thereof, or the presence of nucleic acid encoding ROR1 or the presence of the activity of ROR1 is detected by analysis of a biological sample obtained from said subject.

61. A method according to 60 which includes the step of obtaining said sample for analysis from said subject.

62. A method according to 60 or 61 wherein the sample is a sample of bladder, breast, colorectal, head and neck, liver, lung, ovarian, pancreatic, skin or thyroid tissue.

63. A method according to any one of 57 to 62 wherein the presence of ROR1, or one or more fragments thereof, or the presence of nucleic acid encoding ROR1 or the presence of the activity of ROR1 is detected quantitatively.

64. A method according to 63 wherein the presence of ROR1, or one or more fragments thereof, or the presence of nucleic acid encoding ROR1 or the presence of the activity of ROR1 is detected quantitatively by means involving use of an imaging technology.

65. A method according to any one of 57 to 63 involving use of immunohistochemistry on tissue sections in order to determine the presence of ROR1, or one or more fragments thereof, or the presence of nucleic acid encoding ROR1 or the presence of the activity of ROR1, and thereby to localise bladder cancer, breast cancer, colorectal cancer, head and neck cancer, liver cancer, lung cancer, ovarian cancer, pancreatic cancer, skin cancer or thyroid cancer cells.

66. A method according to any one of 57 to 59 wherein the presence of ROR1, or one or more fragments thereof, or the presence of nucleic acid encoding ROR1 or the presence of the activity of ROR1 is detected by analysis in situ.

67. A method according to any one of 57 to 66 wherein the presence of ROR1 or one or more epitope-containing fragments thereof is detected.

68. A method according to 67 wherein the presence of ROR1 or one or more fragments thereof is detected using an affinity reagent capable of specific binding to ROR1 or one or more fragments thereof.

69. A method according to 68 wherein the affinity reagent is an antibody.

70. A method according to any one of 57 to 66 wherein nucleic acid encoding ROR1 is detected.

71. A method according to 70 wherein nucleic acid encoding ROR1 is detected using a hybridizing agent capable of hybridizing to nucleic acid encoding ROR1.

72. A method according to any one of 57 to 66 wherein the activity of ROR1 is detected.

73. A method of detecting, diagnosing and/or screening for or monitoring the progression of bladder cancer, breast cancer, colorectal cancer, head and neck cancer, liver cancer, lung cancer, ovarian cancer, pancreatic cancer, skin cancer or thyroid cancer or of monitoring the effect of an anti-bladder cancer, anti-breast cancer, anti-colorectal cancer, anti-head and neck cancer, anti-liver cancer, anti-lung cancer, anti-ovarian cancer, anti-pancreatic cancer, anti-skin cancer or anti-thyroid cancer drug or therapy in a subject which comprises detecting the presence or level of antibodies capable of immunospecific binding to ROR1, or one or more epitope-containing fragments thereof or which comprises detecting a change in the level thereof in said subject.

74. A method of detecting, diagnosing and/or screening for bladder cancer, breast cancer, colorectal cancer, head and neck cancer, liver cancer, lung cancer, ovarian cancer, pancreatic cancer, skin cancer or thyroid cancer in a subject which comprises detecting the presence of antibodies capable of immunospecific binding to ROR1, or one or more epitope-containing fragments thereof in said subject, in which (a) the presence of an elevated level of antibodies capable of immunospecific binding to ROR1 or said one or more epitope-containing fragments thereof in said subject as compared with the level in a healthy subject or (b) the presence of a detectable level of antibodies capable of immunospecific binding to ROR1 or said one or more epitope-containing fragments thereof in said subject as compared with a corresponding undetectable level in a healthy subject indicates the presence of bladder cancer, breast cancer, colorectal cancer, head and neck cancer, liver cancer, lung cancer, ovarian cancer, pancreatic cancer, skin cancer or thyroid cancer in said subject.

75. A method of monitoring the progression of bladder cancer, breast cancer, colorectal cancer, head and neck cancer, liver cancer, lung cancer, ovarian cancer, pancreatic cancer, skin cancer or thyroid cancer or of monitoring the effect of an anti-bladder cancer, anti-breast cancer, anti-colorectal cancer, anti-head and neck cancer, anti-liver cancer, anti-lung cancer, anti-ovarian cancer, anti-pancreatic cancer, anti-skin cancer or anti-thyroid cancer drug or therapy in a subject which comprises detecting the presence of antibodies capable of immunospecific binding to ROR1, or one or more epitope-containing fragments thereof in said subject at a first time point and at a later time point, the presence of an elevated or lowered level of antibodies capable of immunospecific binding to ROR1, or one or more epitope-containing fragments thereof in said subject at the later time point as compared with the level in said subject at said first time point, indicating the progression or regression of said cancer or the effect or non-effect of said anti-cancer drug or therapy in said subject.

76. A method according to any one of 73 to 75 wherein the presence of antibodies capable of immunospecific binding to ROR1, or one or more epitope-containing fragments thereof is detected by analysis of a biological sample obtained from said subject.

77. A method according to 76 which includes the step of obtaining said sample for analysis from said subject.

78. A method according to 76 or 77 wherein the sample is a sample of bladder, breast, colorectal, head and neck, liver, lung, ovarian, pancreatic, skin or thyroid tissue.

79. A method according to any one of 57 to 78 wherein the level that may be detected in the candidate subject who has bladder cancer, breast cancer, colorectal cancer, head and neck cancer, liver cancer, lung cancer, ovarian cancer, pancreatic cancer, skin cancer or thyroid cancer is 2 or more fold higher than the level in the healthy subject.

80. A method for killing a cell expressing ROR1 or a fragment thereof comprising contacting said cell with an affinity reagent capable of specific binding to ROR1 or a fragment thereof, wherein said affinity reagent contains or is conjugated to a therapeutic moiety.

81. An method utilising an affinity reagent according to 80 wherein the therapeutic moiety is a cytotoxic moiety or a radioactive isotype.

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

Embodiments of the invention are described herein, which comprise certain elements. The invention also extends to separate embodiments consisting of or consisting essentially of the same elements, and vice versa.

The application of which this description and claims form part may be used as a basis for priority in respect of any subsequent application. The claims of such subsequent application may be directed to any feature or combination of features described herein. They may take the form of product, composition, process, or use claims and may include, by way of example and without limitation, the following claims:

1.-30. (canceled)

31. A method for treating cancer, the method comprising administering to a subject in need thereof a therapeutically effective amount of an affinity reagent which binds to the extracellular domain of receptor tyrosine kinase-like orphan receptor 1 (ROR1).

32. The method according to claim 31, wherein the affinity reagent is an antibody or an antibody mimetic.

33. The method according to claim 32, wherein the affinity reagent contains or is conjugated to a therapeutic moiety.

34. The method according to claim 33, wherein the therapeutic moiety is a cytotoxic moiety or a radioactive isotope.

35. A method for treating a cancer, the method comprising administering to a subject in need thereof a therapeutically effective amount of a hybridizing agent capable of hybridizing to nucleic acid encoding extracellular domain of ROR1, wherein the extracellular domain of ROR1 is expressed in said cancer.

36. The method of claim 35, wherein the hybridizing agent is a short interfering RNA (siRNA), a short hairpin RNA (shRNA), a microRNA (miRNA), and anti-sense nucleic acid or a complementary DNA (cDNA).

37. A method of detecting, diagnosing and/or screening for or monitoring the progression cancer wherein the extracellular domain of ROR1 is expressed in said cancer, or of monitoring the effect of a cancer drug or therapy wherein the extracellular domain of ROR1 is expressed in said cancer, in a subject which comprises detecting the presence or level of the extracellular domain of ROR1, or one or more fragments thereof, or the presence or level of nucleic acid encoding the extracellular domain of ROR1 or which comprises detecting a change in the level thereof in said subject.

38. A method of detecting, diagnosing and/or screening for or monitoring the progression cancer wherein the extracellular domain of ROR1 is expressed in said cancer, or of monitoring the effect of a cancer drug or therapy wherein the extracellular domain of ROR1 is expressed in said cancer, in a subject which comprises detecting the presence or level of antibodies capable of immunospecific binding to the extracellular domain of ROR1, or one or more fragments thereof.

39. A method for identifying an agent for the treatment or prophylaxis of cancer wherein the extracellular domain of ROR1 is expressed in said cancer, wherein the method comprises (a) contacting the extracellular domain of ROR1, or one or more fragments thereof, with a candidate agent; and (b) determining whether the agent binds to the extracellular domain of ROR1, or one or more fragments thereof.

40. An isolated antibody which specifically binds receptor tyrosine kinase-like orphan receptor 1 (ROR1) (SEQ ID NO:1) comprising the complementary determining regions (CDRs) of SEQ ID NO: 22 and SEQ ID NO: 50.

41. One or more isolated nucleic acid(s) encoding the isolated antibody of claim 40.

42. A host comprising the isolated nucleic acid(s) of claim 40.

43. A method of producing the antibody of claim 40, the method comprising culturing the host cell of claim 42 under conditions wherein said antibody is produced.


SEQUENCE LISTING (64 sequences)

SEQ ID NO: 1 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 2 - protein, 12 aa, Homo sapiens
EVVSSTGVLFVK

SEQ ID NO: 3 - protein, 18 aa, Homo sapiens
GTRPPLLALLAALLLAAR

SEQ ID NO: 4 - protein, 8 aa, Homo sapiens
ELPLSAVR

SEQ ID NO: 5 - protein, 8 aa, Homo sapiens
SNPMILMR

SEQ ID NO: 6 - protein, 10 aa, Homo sapiens
SNPMILMRLK

SEQ ID NO: 7 - protein, 17 aa, Homo sapiens
GHLYLPGMDHAQLVAIK

SEQ ID NO: 8 - protein, 378 aa, Homo sapiens
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYI

SEQ ID NO: 9 - protein, 251 aa, Mus musculus
MKQSTIALALLPLLFTPVAKAEVKLVESGGGLVRPGGSLKLSCAVSGFTFSSYAMSWVRQTPEKRLEWVAAINFNRGTTYYSDTVKGRFTISRDNAKNTLYLQLSSLRSEDTAFYYCSRHRYSDYDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 10 - protein, 251 aa, Mus musculus
MKQSTIALALLPLLFTPVAKAEVQLLETGGGLVKPGGSLKLSCAASGFTFSTYAMSWVRQTPEKRLEWVAGINSNRGTTYYPDTVKGRFTISRDNAKNTLSLQMTSLRSEDTALYYCVRHRYTNYDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 11 - protein, 251 aa, Mus musculus
MKQSTIALALLPLLFTPVAKADVMLVESGGGLVKPGGSLKLSCAASGFTFSSYAMSWVRQTPEKRLEWVAAININRGTTYYSDTVKGRFTISRDNAKNTLYLQLSSLRSEDTALYYCSRHRYSDYDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 12 - protein, 250 aa, Mus musculus
MKQSTIALALLPLLFTPVAKAEVKLVESGGGLVKPGGSLKLSCAASGFTFSNYGMSWVRQTPERRLEWVAAMNNNGASTYYPDTVKGRFTISRDNAKNTLYLQMSSLRSEDTALYFCVRHNNYVDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 13 - protein, 249 aa, Mus musculus
MKQSTIALALLPLLFTPVAKAEVKLVESGGGLVKPGGSLKLSCAASGFTFSNYDMSWVRQSPEKRLEWVAAINRKGHSTYYPDTVQGRFTISRDNAKNTLYLQMSSLRSEDTALYYCVRLDDNYYFFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 14 - protein, 251 aa, Mus musculus
MKQSTIALALLPLLFTPVAKAEVMLVESGGGLVKPGGSLKLSCAASGFTFSPYAMSWVRQTPEKRLEWVAAINSNRGTTYYPDTVKGRFTISRDNAKNTLYLQMSSLRSEDTAFYYCVRHRYNNYDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 15 - protein, 251 aa, Mus musculus
MKQSTIALALLPLLFTPVAKAEVMLVESGGGLVKPGGSLKISCAASGFSFSSYAMSWVRQTPEKSLEWVAAININRGTPYYPDTVKGRFTISRDNAKNTLYLQMSSLRSEDTALYYCVRHRNSNNDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 16 - protein, 175 aa, Mus musculus
MKQSTIALALLPLLFTPVAKADVQVVESGGGLVKPGGSLKLSCAASGFTFSSYAMSWVRQTPEKRLEWVAAINPNGGSTYYPDTVKGRFTISRDNAKNTLYLQMSGLRSEDTALYYCARLPWSPYTLDYWGQGTSVTVSSAKTTPPSVYPLAPGSAAQTNSMVTLGCLVKGYFPE

SEQ ID NO: 17 - protein, 251 aa, Mus musculus
MKQSTIALALLPLLFTPVAKAEVQLVETGGDLVKPGGSLKLSCVASGFTFSSNAMSWVRQTPEKRLEWVAAINSKGGGTYYPDTVRGRFTISRDNAKNTLYLQVTSLRSEDTALYYCVSHGDNKYFYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 18 - protein, 251 aa, Mus musculus
MKQSTIALALLPLLFTPVAKAEVQLVETGGGLVKPGGSLKLSCAASGFAFSSYAMSWVRQTPEKRLEWVAAINNRGGGTYYPDTVRGRFTISRDNAKNTLYLQMSSLRSADTALYYCVRHDNLNYDYAMDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 19 - protein, 249 aa, Mus musculus
MKQSTIALALLPLLFTPVPKAEVQLVESGGDLVKPGGSLKLSCAASGFTFSRYGMSWVRQTPEKRLEWVAAINPNGGTTYYPDTVKGRFTISRDNAKNTLFLQMTGLRSEDTALYYCARLPWSPYTLDYWGQGTSVIVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 20 - protein, 251 aa, Mus musculus
MKQSTIALALLPLLFTPVAKAEVQLVESGGGLVKPGGSLKLSCAASGFTFSSYAMSWVRQTPEKRLEWVAAINSNRGTTYYSDTVKGRFTISRDNAKNTLYLQMSSLRSEDTAFYYCTRHRYSDYDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 21 - protein, 251 aa, Mus musculus
MKQSTIALALLPLLFTPVAKAEVQLVETGGGLVKPGGSLKLSCAASGFTFSSYAMSWIRQTPEKRLEWVAGINSNRGTTYYPDTVKGRFTISRDNAKNTLYLQMNSLRSEDSALYYCVRHRYIDYDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 22 - protein, 251 aa, Mus musculus
MKQSTIALALLPLLFTPVAKAQVQLKQSGAELVKPGASVKISCKATGYTFSSYWIEWVKERPGHGLEWIGEILPGIGNTNYNEKFKGKATFTADLSSKTAYMQLSSLTSEDSAVYYCASGGYSTVYWYFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCHHHHHHH

SEQ ID NO: 23 - DNA, 814 bp, Mus musculus
ttacccacgctttgtacatggagaaaataaagtgaaacaaagcactattgcactggcactcttaccgctcttatttacccctgtggcaaaagccgaggtgaagctggtggaatctgggggaggcttagtgaggcctggagggtccctgaaactctcctgtgcagtctctggattcactttcagtagctatgccatgtcttgggttcgccagactccggagaagaggctggaatgggtcgcagccattaattttaatcgtggtaccacctactattcagacactgtgaagggccgattcaccatctccagagacaatgccaagaataccctgtacctgcaactgagcagtctgaggtctgaggacacagccttttattactgttcaagacaccgctatagtgactacgactatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaaatggacagcttaatcatttataaagct

SEQ ID NO: 24 - DNA, 838 bp, Mus musculus
aaccctggcgttacccacgctttgtacatggagaaaataaagtgaaacaaagcactattgcactggcactcttaccgctcttatttacccctgtggcaaaagccgaagtgcagctgttggagactgggggaggcttagtgaagcctggagggtccctgaaactctcctgtgcagcctctggattcactttcagtacctatgccatgtcttgggttcgccagactccggagaagaggctggagtgggtcgcaggcattaatagtaatcgtggtaccacctactatccagacactgtgaagggccgcttcaccatctccagagacaatgccaagaacaccctgtccctgcaaatgaccagtctgaggtctgaggacacagccttgtattattgtgtaagacaccgctatactaactacgactatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatcatcgataagctttaatgcggtagtttat

SEQ ID NO: 25 - DNA, 864 bp, Mus musculus
cgtcgttttacaacgtcgtgactgggaaaaccctggcgttacccacgctttgtacatggagaaaataaagtgaaacaaagcactattgcactggcactcttaccgctcttatttacccctgtggcaaaagccgatgtgatgctggtggagtctgggggaggcttagtgaagcctggagggtccctgaaactctcctgtgcagcctctggattcactttcagtagctatgccatgtcttgggttcgccagactccggagaagaggctggaatgggtcgcagccattaatattaatcgtggtaccacctactattcagacactgtgaagggccgattcaccatctccagagacaatgccaagaataccctgtacctgcaactgagcagtctgaggtctgaggacacagccttgtattactgttcaagacaccgctatagtgactacgactatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatcatcgataagctttaatgcggtagttt

SEQ ID NO: 26 - DNA, 837 bp, Mus musculus
ttacccacgctttgtacatggagaaaataaagtgaaacaaagcactattgcactggcactcttaccgctcttatttacccctgtggcaaaagccgaggtgaagctggtggaatctgggggaggcttagtgaagcctggagggtccctgaaactctcctgtgcagcctctggattcactttcagtaactatggcatgtcttgggttcgccagactccggagaggaggctggagtgggtcgcagccatgaataataatggtgctagcacctactatccagacactgtgaagggccgattcaccatctccagagacaatgccaagaacaccctgtacctgcaaatgagcagtctgaggtctgaggacacagccttgtatttctgtgtaagacataataactacgttgactatgctatggactattggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatcatcgataagctttaatgcggtagtttatcacagttaaatt

SEQ ID NO: 27 - DNA, 831 bp, Mus musculus
accctggcgttacccacgctttgtacatggagaaaataaagtgaaacaaagcactattgcactggcactcttaccgctcttatttacccctgtggcaaaagccgaggtgaagctggtggaatctgggggaggcttagtgaagcctggagggtccctgaaactctcctgtgcagcctctggattcactttcagtaactatgacatgtcttgggttcgccagagtccggagaagaggctggagtgggtcgcagccattaatcgtaaaggtcatagtacctactatccagacactgtgcagggccgattcaccatctccagagacaatgccaagaacaccctgtacctgcaaatgagcagtctgaggtctgaggacacagccttgtattactgtgtaagacttgacgataactactacttctttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatcatcgataagctttaatgcggtagtttat

SEQ ID NO: 28 - DNA, 848 bp, Mus musculus
ggaaaaccctggcgttacccacgctttgtacatggagaaaataaagtgaaacaaagcactattgcactggcactcttaccgctcttatttacccctgtggcaaaagccgaagtgatgctggtggagtctgggggaggcttagtgaagcctggagggtccctgaaactctcctgcgcagcctctggattcactttcagtccctatgccatgtcttgggttcgccagactccggagaagaggctggagtgggtcgcagccattaatagtaatcgtggtaccacctactatccagacactgtgaagggccgattcaccatctccagagacaatgccaagaacaccctgtacctgcaaatgagcagtctgaggtctgaggacacagccttttattactgtgtaagacaccgctataataactacgactatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatcatcgataagctttaatgcggtagtttatcacagt

SEQ ID NO: 29 - DNA, 851 bp, Mus musculus
cgttttacaacgtcgtgactgggaaaaccctggcgttacccacgctttgtacatggagaaaataaagtgaaacaaagcactattgcactggcactcttaccgctcttatttacccctgtggcaaaagccgaagtgatgctggtggagtctgggggaggcttagtgaagcctggagggtccctgaaaatctcctgtgcagcctctggattctctttcagtagctatgccatgtcttgggttcgccagactccggagaagagcctggaatgggtcgcagccattaatattaatcgtggtaccccctattatccagacactgtgaagggccgattcaccatctccagagacaatgccaagaacaccctgtacctgcaaatgagtagtctgaggtctgaggacacagccttgtattactgtgtaagacaccgcaatagtaacaacgactatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatcatcgataagctttaat

SEQ ID NO: 30 - DNA, 587 bp, Mus musculus
tacaacgtcgtgactgggaaaaccctggcgttacccacgctttgtacatggagaaaataaagtgaaacaaagcactattgcactggcactcttaccgctcttatttacccctgtggcaaaagccgacgtgcaggtggtggagtctgggggaggcttagtgaagcctggagggtccctgaaactctcctgtgcagcctctggattcactttcagtagctatgccatgtcttgggttcgccagactccggagaagaggctggagtgggtcgcagccattaatcctaatggtggtagtacctactatccagacactgtgaagggccgattcaccatctccagagacaatgccaagaacaccctatacctgcaaatgagcggtctgaggtctgaggacacagccttgtattactgtgcaagactcccatggtccccctatactttggactactggggtcaaggaacctcagtcaccgtctcctcagccaaaacgacacccccatctgtctatccactggcccctggatctgctgcccaaactaactccatggtgaccctgggatgcctggtcaagggctatttccctgagc

SEQ ID NO: 31 - DNA, 800 bp, Mus musculus
ttacccacgctttgtacatggagaaaataaagtgaaacaaagcactattgcactggcactcttaccgctcttatttacccctgtggcaaaagccgaagtgcagcttgtggagactgggggagacttagtgaagcctggagggtccctgaaactctcctgtgtagcctctggattcactttcagtagcaatgccatgtcctgggttcgccagactccggagaagaggctggagtgggtcgcagccattaatagtaaaggtggtggcacctactatccagacactgtgaggggccgattcaccatctccagagacaatgccaagaacaccctgtacctgcaagtgaccagtctgaggtctgaggacacagccttgtattactgtgtaagccatggggataataagtacttttatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatc

SEQ ID NO: 32 - DNA, 815 bp, Mus musculus
ttacccacgctttgtacatggagaaaataaagtgaaacaaagcactattgcactggcactcttaccgctcttatttacccctgtggcaaaagccgaagtgcagcttgtggagactgggggaggcttagtgaagcctggagggtccctgaaactctcctgtgcagcctctggattcgctttcagtagctatgccatgtcttgggttcgccaaactccggagaagaggctggagtgggtcgcagccattaataatagaggtggtggcacctactatccagacactgtgaggggccgattcaccatctccagagacaatgccaagaacaccctgtacctgcaaatgagcagcctgaggtctgcggacacagccttgtattactgtgtgagacatgacaatcttaactatgactatgctatggactcctggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatcatcgataagctttaa

SEQ ID NO: 33 - DNA, 858 bp, Mus musculus
cgtcgtgactgggaaaaccctggcgttacccacgctttgtacatggagaaaataaagtgaaacaaagcactattgcactggcactcttaccgctcttatttacccctgtgccaaaagccgaagtgcagctggtggagtctgggggagacttagtgaagcctggagggtccctgaaactctcctgtgcagcctctggattcactttcagtagatatggcatgtcttgggttcgccagactccggagaagaggctggagtgggtcgcagccattaatcctaatggtggtactacctactatccagacactgtgaagggccgattcaccatctcccgagacaatgccaagaacaccctgttcctgcaaatgaccggtctgaggtctgaggacacagccttatactactgtgcaagactcccatggtccccctatactttggactactggggtcaaggaacctcagtcatcgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatcatcgataagctttaatgcggtagtttatcacagttaaat

SEQ ID NO: 34 - DNA, 849 bp, Mus musculus
gcgttacccacgctttgtacatggagaaaataaagtgaaacaaagcactattgcactggcactcttaccgctcttatttacccctgtggcaaaagccgaagtgcagctggtggagtctgggggaggcttagtgaagcctggagggtccctgaaactctcctgcgcagcctctggattcactttcagtagctatgccatgtcttgggttcgccagactccggagaagaggctagagtgggtcgcagccattaatagtaatcgtggtaccacctactattcagacactgtgaagggccgattcaccatctccagagacaatgccaagaacaccctgtacctgcaaatgagcagtctgaggtctgaggacacagccttctattactgtacaagacaccgctatagtgactacgactatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatcatcgataagctttaatgcggtagtttatcacagttaaattgctacg

SEQ ID NO: 35 - DNA, 829 bp, Mus musculus
ggcgttacccacgctttgtacatggagaaaataaagtgaaacaaagcactattgcactggcactcttaccgctcttatttacccctgtggcaaaagccgaagtgcagcttgtggagactgggggaggcttagtgaagcctggagggtccctgaaactctcctgtgcagcctctggattcactttcagtagctatgccatgtcttggattcgccagactccggagaagaggctggagtgggtcgcaggcattaatagtaatcgtggtaccacctactatccagacactgtgaagggccgattcaccatctccagagacaatgccaagaacaccctgtacctgcaaatgaacagtctgaggtctgaggactcagccttgtattactgtgtaagacaccgctatattgactacgactatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaattgacagcttatcatcgataagctttaatgcggtagtt

SEQ ID NO: 36 - DNA, 846 bp, Mus musculus
gtcgtgactgggaaaaccctggcgttacccacgctttgtacatggagaaaataaagtgaaacaaagcactattgcactggcactcttaccgctcttatttacccctgtggcaaaagcccaggtgcagcttaagcagtctggggctgagctggtgaagcctggggcctcagtgaagatatcctgcaaggctactggctacacattcagtagttactggatagagtgggtaaaggagaggcctggacatggccttgagtggattggagagattttacctggaattggtaatactaactacaatgagaaattcaagggcaaggccacattcactgctgatctatcctccaagacagcctacatgcaactcagcagcctgacatctgaggactctgccgtctattactgtgcaagtggggggtatagtaccgtctattggtattttgatgtctggggcgcagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtcatcatcaccatcaccatcactaatttgacagctttaatcattcaattaagcttttaat

SEQ ID NO: 37 - protein, 236 aa, Mus musculus
MKYLLPTAAAGLLLLAAQPAMADIVMSQSPSSMYASLGERVTITCKASQDINSYLNWFQQKPGKSPKTLIYRANRLVDGVPSRFSGSGSGHDYFLTIRSLEYEDMGIYYCLQYDEFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 38 - protein, 236 aa, Mus musculus
MKYLLPTAAAGLLLLAAQPAMADIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 39 - protein, 236 aa, Mus musculus
MKYLLPTAAAGLLLLAAQPAMADIQLTQSPSSMYASLGERVTIACKASQDINSYLSWFQQKPGKSPKTLIHRANRLVDGVPSRFSGSGSGQDYSLTISSLEYEDIGIYYCLQYDEFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 40 - protein, 236 aa, Mus musculus
MKYLLPTAAAGLLLLAAQPAMADILLTQSPSSMYTSLGERVTITCKASQDINSYLSWFQQKPGKSPKTLIYRANKLVDGVPSRFSGSGSGQDYSLTISSLESEDMGIYYCLQYDEFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 41 - protein, 236 aa, Mus musculus
MKYLLPTAAAGLLLLAAQPAMADIKMTQSPSSMYASLGERVTITCKASQDINSYLSWFQQKPGKSPKTLIYRAKRLIDGVPSRFSGSGSGQDYSLTISSLEYEDMGIYYCLQYDEFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 42 - protein, 236 aa, Mus musculus
MKYLLPTAAAGLLLLAAQPAMADIVMSQSPSSMYASLGERVTITCKASQDINSYLSWFQQKPGKSPKTLTYRANRLVEGVPSRFSGSGSGQDYSLTISSLEYEDMGIYYCLQYDEFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 43 - protein, 236 aa, Mus musculus
MKYLLPTAAAGLLLLAAQPAMADIVMTQSPSSMYTSLGERVTITCKASQDINSYLSWFQQKPGKSPKTLIYRANRLIDGVPSRFSGSGSGQDYSLTISSLEYEDMGIYYCLQYDEFPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 44 - protein, 236 aa, Mus musculus
MKYLLPTAAAGLLLLAAQPAMANIVMTQSPVSLSMAIGEKVTIRCITNTDIDDAMNWYQQKPGEPPKLLISEGNTLRPGVPSRFSSSGYGTDFVFTIENMLSEDVADYYCLQTDNLPLTFGSGTKLAIKRADAAPTVSIFPPSSEQLTSGGASVVCFLNNFYPKDINVKWKIDGSERQNGVMNSWTDQDSKDSTYSMSSTLTLTKDEYERHNSYTCEATHKTSTSPIVKSFNRNES

SEQ ID NO: 45 - protein, 236 aa, Mus musculus
MKYLLPTAAAGLLLLAAQPAMANIVMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 46 - protein, 236 aa, Mus musculus
MKYLLPTAAAGLLLLAAQPAMANIVMTQSPSSMYASLGERVTITCKASQDIYSYLSWFQQKPGKSPKTLIYRANRLVDGVPSRFSGSGSGQDYSLTISSLDYEDVGIYYCLQYDEFPYTFGSGTKLEIERADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 47 - protein, 236 aa, Mus musculus
MKYLLPTAAAGLLLLAAQPAMAETTVTQSPVSLSMAIGEKVTIRCMTSTDIDDALNWYQQKPGEPPKLLISEGNSLRPGVPSRFSSSGNGTDFVFTIENMLSEDVADYYCLQSDNLPLTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 48 - protein, 236 aa, Mus musculus
MKYLLPTAAAGLLLLAAQPAMADIKMTQSPSSMYASLGERVTITCKASQDINSYLSWFQQKPGKSPMTLTHRANRLVDGVPSRFSGSGSGQDYSLTISSLENEDMGIYYCLQYDEFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 49 - protein, 236 aa, Mus musculus
MKYLLPTAAAGLLLLAAQPAMANIVMTQSPSSMYASLGERVTIICKSSQDINSYLSWFQQKPGKSPKTLIFRANRLVDGVPSRFSGSGSGQDYSLTISSLEYEDMGIYYCLQYDEFPYTFGGGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 50 - protein, 242 aa, Mus musculus
MKYLLPTAAAGLLLLAAQPAMADVVMSQSPSSLAVSTGEKVTLSCKSSQSLLNSRTRKNYLAWYQQKPGQSPKLLIYWTSTRESGVPNRFTGSGSGTDFTLTISSVQAEDLAVYYCKQSYDLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 51 - DNA, 809 bp, Mus musculus
tatcgcaactctctactgtttctccatacccgtttttttggatggagtgaaacgatgaaatacctattgcctacggcagccgctggattgttattactcgctgcccaaccagccatggccgacatcgttatgtctcagtctccatcttccatgtatgcatctctaggagagagagtcactatcacttgcaaggcgagtcaggacattaatagctatttaaactggttccagcagaaaccaggcaaatctcctaagaccctgatctatcgtgcaaacagattggtagatggggtcccatcaaggttcagtggcagtggatctgggcacgattattttcttaccattcgcagcctggaatatgaagatatgggaatttattattgtctacagtatgatgagtttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcg

SEQ ID NO: 52 - DNA, 835 bp, Mus musculus
ttatcgcaactctctactgtttctccatacccgtttttttggatggagtgaaacgatgaaatacctattgcctacggcagccgctggattgttattactcgctgcccaaccagccatggccgacatcaaaatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgaatttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcgttttacaacgtcgtgactgggaaaa

SEQ ID NO: 53 - DNA, 822 bp, Mus musculus
tatcgcaactctctactgtttctccatacccgtttttttggatggagtgaaacgatgaaatacctattgcctacggcagccgctggattgttattactcgctgcccaaccagccatggccgacattcagctgacccagtctccatcttccatgtatgcatctctaggagagagagtcactatcgcttgcaaggcgagtcaggacattaatagctatttaagctggttccagcagaaaccagggaaatctcctaagaccctgatccatcgtgcaaacagattggtagatggggtcccatcaaggttcagtggcagtggatctgggcaagattattctctcaccatcagcagcctggagtatgaagatatcggaatttattattgtctacagtatgatgagtttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcgttttacaacgtcg

SEQ ID NO: 54 - DNA, 795 bp, Mus musculus
tcgcaactctctactgtttctccatacccgtttttttggatggagtgaaacgatgaaatacctattgcctacggcagccgctggattgttattactcgctgcccaaccagccatggccgacatcttgctgactcagtctccatcttccatgtatacatctctaggagagagagtcactatcacttgcaaggcgagtcaggacattaatagctatttaagctggttccagcagaaaccaggaaaatctcctaagaccctgatctatcgtgcaaacaaattggtagatggggtcccatcaagattcagtggcagtggatctgggcaagattattctctcaccatcagcagcctggagtctgaagatatgggaatttattattgtctacagtatgatgagtttccgtacacgttcggaggggggaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttgg

SEQ ID NO: 55 - DNA, 820 bp, Mus musculus
tcgcaactctctactgtttctccatacccgtttttttggatggagtgaaacgatgaaatacctattgcctacggcagccgctggattgttattactcgctgcccaaccagccatggccgacatcaaaatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaagagattgatagatggggtcccatcaaggttcagtggcagtggatctgggcaagattattctctcaccatcagcagcctggagtatgaagatatgggaatttattattgtctacagtatgatgagtttccttacacgttcggaggggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcgttttacaacgtcg

SEQ ID NO: 56 - DNA, 823 bp, Mus musculus
ttttatcgcaactctctactgtttctccatacccgtttttttggatggagtgaaacgatgaaatacctattgcctacggcagccgctggattgttattactcgctgcccaaccagccatggccgacatcgttatgtctcagtctccatcttccatgtatgcatctctaggagagagagtcactatcacttgcaaggcgagtcaagacattaatagctatttaagctggttccagcagaaaccagggaaatctcctaagaccctgacctatcgtgcaaacagattggtagaaggggtcccatcaaggttcagtggcagtggatctgggcaagattattctctcaccatcagcagcctggaatatgaagatatgggaatttattattgtctacagtatgatgagtttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcgttttacaacgt

SEQ ID NO: 57 - DNA, 837 bp, Mus musculus
gcaactctctactgtttctccatacccgtttttttggatggagtgaaacgatgaaatacctattgcctacggcagccgctggattgttattactcgctgcccaaccagccatggccgacattgtgatgacccagtctccatcttccatgtatacatctctaggagagagagtcactatcacttgcaaggcgagtcaggacattaatagctatttaagctggttccagcagaaaccagggaaatctcctaagaccctgatctatcgtgcaaacagattgatagatggggtcccatcaaggttcagtggcagtggatctgggcaagattattctctcaccatcagcagcctggagtatgaagatatgggaatttattattgtctacagtatgatgagtttccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcgttttacaacgtcgtgactgggaaaaccctggc

SEQ ID NO: 58 - DNA, 729 bp, Mus musculus
gagtgaaacgatgaaatacctattgcctacggcagccgctggattgttattactcgctgcccaaccagccatggccaacatcgttatgacccagtctccagtatccctgtccatggctataggagaaaaagtcaccatcagatgcataaccaacactgatattgatgatgctatgaactggtaccagcaaaagccaggggaacctcctaagctccttatttcagaaggcaatactcttcgtcctggagtcccatcccgattctccagcagtggctatggtacagattttgtttttacaattgaaaacatgctctcagaagatgttgcagattactactgtttgcaaactgataacttgcctctcacgttcggctcggggacaaagttggcaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcatgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattag

SEQ ID NO: 59 - DNA, 814 bp, Mus musculus
tcgcaactctctactgtttctccatacccgtttttttggatggagtgaaacgatgaaatacctattgcctacggcagccgctggattgttattactcgctgcccaaccagccatggccaacatcgttatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgtacacgttcggaggggggaccaaactggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcgttttaca

SEQ ID NO: 60 - DNA, 810 bp, Mus musculus
gcaactctctactgtttctccatacccgtttttttggatggagtgaaacgatgaaatacctattgcctacggcagccgctggattgttattactcgctgcccaaccagccatggccaacatcgttatgacccagtctccatcttccatgtatgcatctctaggagagagggtcactatcacttgcaaggcgagtcaggacatttatagctatttaagctggttccagcagaaaccaggcaaatctcctaagaccctgatctatcgtgcaaacagattggtagatggggtcccatcaaggttcagtggcagtggatctgggcaagattattctctcaccatcagcagcctggactatgaagatgtgggaatttattattgtctacagtatgatgagtttccgtacacgttcggctcggggacaaagttggaaatagaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcgtttta

SEQ ID NO: 61 - DNA, 823 bp, Mus musculus
cgcaactctctactgtttctccatacccgtttttttggatggagtgaaacgatgaaatacctattgcctacggcagccgctggattgttattactcgctgcccaaccagccatggccgaaacaactgtgacccagtctccagtatccctgtccatggctataggagaaaaagtcaccatcagatgcatgaccagcactgatattgatgatgctctgaactggtaccagcaaaagccaggggaacctcctaaactccttatttcagaaggcaatagtcttcgtcctggagtcccatcccgattctccagcagtggcaatggtacagattttgtttttacaattgaaaacatgctctcagaagatgttgcagattactactgtttgcaaagtgataacttgcctctcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaatgcgtcacttggcactggccgtcgttttacaacgtcgtgac

SEQ ID NO: 62 - DNA, 823 bp, Mus musculus
tatcgcaactctctactgtttctccatacccgtttttttggatggagtgaaacgatgaaatacctattgcctacggcagccgctggattgttattactcgctgcccaaccagccatggccgacatcaaaatgacccagtctccatcttccatgtatgcatctctaggagagagagtcactatcacttgcaaggcgagtcaggacattaatagctatttaagctggttccagcagaaaccagggaaatctcctatgaccctgacccatcgtgcaaacagattggtagatggggtcccatcaaggttcagtggcagtggatctgggcaagattattctctcaccatcagcagcctggagaatgaagatatgggaatttattattgtctacagtatgatgagtttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcgttttacaacgtcgt

SEQ ID NO: 63 - DNA, 822 bp, Mus musculus
cgcaactctctactgtttctccatacccgtttttttggatggagtgaaacgatgaaatacctattgcctacggcagccgctggattgttattactcgctgcccaaccagccatggccaacatcgttatgacccagtctccatcttccatgtatgcatctctaggagagagagtcactatcatttgcaagtcgagtcaggacattaatagctatttaagttggttccagcagaaaccagggaagtctcctaagaccctgatctttcgtgcaaacagattggtagatggggtcccatcaaggttcagtggcagtggatctgggcaagattattctctcaccatcagcagcctggagtatgaagatatgggaatttattattgtctacagtatgatgagtttccgtacacgttcggaggggggaccaagctggaagtaaaacgggctgatgctgcaccaaccgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcgttttacaacgtcgtga

SEQ ID NO: 64 - DNA, 826 bp, Mus musculus
tcgcaactctctactgtttctccatacccgtttttttggatggagtgaaacgatgaaatacctattgcctacggcagccgctggattgttattactcgctgcccaaccagccatggccgacgttgtgatgtcacagtctccatcctccctggctgtgtcaacaggagagaaggtcactttgagctgcaaatccagtcagagtctgctcaacagtagaacccgaaagaactacttggcttggtaccagcagaaaccagggcagtctcctaaactgctgatctactggacatccactagggaatctggggtccctaatcgcttcacaggcagtggatctgggacagatttcactctcaccatcagcagtgtgcaggctgaagacctggcagtttattactgcaagcaatcttatgatcttccgtggacgttcggtgggggcaccaaactggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtcttaagtgattagctaattctagaacgcgtcacttggcactggccgtcgt